SEQUENCE LISTING (19 sequences)

SEQ ID NO: 1 - protein, 245 aa, Artificial Sequence
  RBD of Spike protein from SARS-CoV-2
MDAMKRGLCCVLLLCGAVFVSPRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 473 aa, Artificial Sequence
  Fusion protein RBD and C3d-p28.6
MDAMKRGLCCVLLLCGAVFVSPRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGGSGGGGSKFLTTAKDKNRWEDPGKQLYNVEATSYAGGGGSGGGGSKFLTTAKDKNRWEDPGKQLYNVEATSYAGGGGSGGGGSKFLTTAKDKNRWEDPGKQLYNVEATSYAGGGGSGGGGSKFLTTAKDKNRWEDPGKQLYNVEATSYAGGGGSGGGGSKFLTTAKDKNRWEDPGKQLYNVEATSYAGGGGSGGGGSKFLTTAKDKNRWEDPGKQLYNVEATSYA

SEQ ID NO: 3 - DNA, 738 bp, Artificial Sequence
  DNA equivalent of S-RBD
atggatgctatgaagaggggcctgtgctgcgtgctgcttctgtgtggcgctgtgttcgtgtcccctagagtgcagcctaccgagagcatcgtgcggttccccaacatcaccaatctgtgccctttcggcgaggtgttcaacgccaccagattcgcctctgtgtacgcctggaaccggaagcggatcagcaattgcgtggccgactacagcgtgctgtacaacagcgccagcttcagcaccttcaagtgctacggcgtgtcacccaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccggaagtccaacctgaagcctttcgagcgggacatcagcaccgagatctatcaggccggcagcaccccttgcaatggcgtggaaggcttcaactgctacttcccactgcagtcctacggcttccagcctacaaacggcgtgggctaccagccttacagagtggtggtgctgagcttcgagctgctgcatgctcctgccacagtgtgcggccctaagaaaagcaccaacctggtcaagaacaaatgcgtgaacttctga

SEQ ID NO: 4 - DNA, 1422 bp, Artificial Sequence
  DNA equivalent of S-RBD-p28.6
atggatgctatgaagaggggcctgtgctgcgtgctgcttctgtgtggcgctgtgttcgtgtcccctagagtgcagcctaccgagagcatcgtgcggttccccaacatcaccaatctgtgccctttcggcgaggtgttcaacgccaccagattcgcctctgtgtacgcctggaaccggaagcggatcagcaattgcgtggccgactacagcgtgctgtacaacagcgccagcttcagcaccttcaagtgctacggcgtgtcacccaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccggaagtccaacctgaagcctttcgagcgggacatcagcaccgagatctatcaggccggcagcaccccttgcaatggcgtggaaggcttcaactgctacttcccactgcagtcctacggcttccagcctacaaacggcgtgggctaccagccttacagagtggtggtgctgagcttcgagctgctgcatgctcctgccacagtgtgcggccctaagaaaagcaccaacctggtcaagaacaaatgcgtgaacttcggcggaggcggaagtggtggcggcggatctaagtttctgaccaccgccaaggacaagaacagatgggaagatcccggcaagcagctgtacaatgtggaagccacaagctacgcaggcggcggaggaagcggaggcggaggtagtaaatttctgacaacggctaaagataagaatcgctgggaagatcctgggaaacagctctataacgtcgaggccaccagctatgctggcggtggcggatctggcggcggtggttcaaaattcctgactacagccaaggataagaatcgttgggaagatccaggcaagcaactctataatgttgaggctacctcttacgctggtggcggaggttctggcggcggaggctctaaatttctcacaacagcaaaggacaagaatcgatgggaagatccgggaaaacaactgtacaacgttgaggcaacatcctatgcaggcggaggtggcagtggcggaggtggaagcaagtttctgactactgcaaaagataagaatagatgggaagatcccgggaagcaactctacaacgtcgaagctactagttatgccggtggcggtggatctggcggaggcggcagcaaattcctgaccaccgctaaagacaagaatcgttgggaagatcccggaaagcagttgtataacgttgaagctacgtcctacgcctga

SEQ ID NO: 5 - DNA, 1260 bp, Artificial Sequence
  DNA equivalent of SARS-CoV-2 nucleocapsid
atgagcgacaacggccctcagaaccagagaaacgcccctcggatcacatttggcggccctagcgatagcaccggcagcaatcagaatggcgagagaagcggcgccagaagcaagcagagaaggcctcaaggcctgcctaacaacaccgccagctggttcacagccctgacacagcacggcaaagaggacctgaagttccctagaggacagggcgtgcccatcaacaccaacagcagccccgatgaccagatcggctactacagacgggccaccagaagaatcagaggcggcgacggcaagatgaaggatctgagccccagatggtacttctactacctcggcacaggacccgaagccggacttccttatggcgccaacaaggacggcatcatctgggttgcaacagaaggcgccctgaacacccctaaggaccacatcggcaccagaaatcccgccaacaatgccgccattgtgctgcagttgcctcagggcacaacactgcccaagggcttttacgccgagggctctagaggcggatctcaggccagcagcagaagcagctccagatccagaaacagctcccggaatagcacccctggctccagcagaggaacaagccctgctagaatggccggcaacggcggagatgctgctctggcacttctcctgctggaccggctgaatcagctggaaagcaagatgagcggcaagggacagcagcagcagggccagaccgtgacaaaaaagtctgccgccgaggccagcaagaagcccagacagaaaagaaccgccaccaaggcctacaacgtgacccaggcctttggcagaagaggccctgagcagacccagggcaatttcggcgatcaagagctgatcagacagggcaccgactacaagcactggcctcagatcgcccagtttgccccatctgccagcgcctttttcggcatgagccggatcggcatggaagtgacacctagcggcacctggctgacatacacaggcgccatcaagctggacgacaaggaccccaacttcaaggaccaagtgatcctgctgaacaagcacatcgacgcctacaagacattccctccaaccgagcctaagaaggacaagaagaagaaggccgacgagacacaggccctgcctcagcgccagaaaaagcagcagacagtgacactgctgccagccgccgacctggacgatttttctaagcagctgcagcagagcatgagcagcgccgattctacacaggcctga

SEQ ID NO: 6 - DNA, 669 bp, Artificial Sequence
  DNA equivalent of SARS-CoV-2 membrane protein
atggccgatagcaatggcaccatcaccgtggaagaactgaagaaactgctggaacagtggaacctcgtgatcggcttcctgttcctgacctggatctgcctgctgcagttcgcctacgccaaccggaacagattcctgtatattatcaagctgatcttcctgtggctgctgtggcccgtgacactggcctgttttgtgctggccgccgtgtaccggatcaactggatcacaggcggaatcgccattgccatggcctgtctcgttggcctgatgtggctgagctactttatcgccagcttccggctgttcgcccggaccagatccatgtggtccttcaatcccgagacaaacatcctgctgaacgtgcccctgcacggcaccatccttacaagacctctgctggaaagcgagctggtcatcggagccgtgatcctgagaggccacctgagaattgccggacaccacctgggcagatgcgacatcaaggacctgcctaaagaaatcacagtggccaccagcagaaccctgtcctactataagctgggcgccagccagagagtggccggcgattctggatttgccgcctacagcagataccggatcggcaactacaagctgaacaccgaccacagctccagcagcgacaatatcgcactgctggtgcagtga

SEQ ID NO: 7 - DNA, 9372 bp, Artificial Sequence
  DNA equivalent of RNA replicon encoding SARS- CoV-2 S-RBD-C3d-p28.6
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggctgttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggatgctatgaagaggggcctgtgctgcgtgctgcttctgtgtggcgctgtgttcgtgtcccctagagtgcagcctaccgagagcatcgtgcggttccccaacatcaccaatctgtgccctttcggcgaggtgttcaacgccaccagattcgcctctgtgtacgcctggaaccggaagcggatcagcaattgcgtggccgactacagcgtgctgtacaacagcgccagcttcagcaccttcaagtgctacggcgtgtcacccaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccggaagtccaacctgaagcctttcgagcgggacatcagcaccgagatctatcaggccggcagcaccccttgcaatggcgtggaaggcttcaactgctacttcccactgcagtcctacggcttccagcctacaaacggcgtgggctaccagccttacagagtggtggtgctgagcttcgagctgctgcatgctcctgccacagtgtgcggccctaagaaaagcaccaacctggtcaagaacaaatgcgtgaacttcggcggaggcggaagtggtggcggcggatctaagtttctgaccaccgccaaggacaagaacagatgggaagatcccggcaagcagctgtacaatgtggaagccacaagctacgcaggcggcggaggaagcggaggcggaggtagtaaatttctgacaacggctaaagataagaatcgctgggaagatcctgggaaacagctctataacgtcgaggccaccagctatgctggcggtggcggatctggcggcggtggttcaaaattcctgactacagccaaggataagaatcgttgggaagatccaggcaagcaactctataatgttgaggctacctcttacgctggtggcggaggttctggcggcggaggctctaaatttctcacaacagcaaaggacaagaatcgatgggaagatccgggaaaacaactgtacaacgttgaggcaacatcctatgcaggcggaggtggcagtggcggaggtggaagcaagtttctgactactgcaaaagataagaatagatgggaagatcccgggaagcaactctacaacgtcgaagctactagttatgccggtggcggtggatctggcggaggcggcagcaaattcctgaccaccgctaaagacaagaatcgttgggaagatcccggaaagcagttgtataacgttgaagctacgtcctacgcctgaggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaataggg

SEQ ID NO: 8 - protein, 419 aa, Severe acute respiratory syndrome-related coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 222 aa, Severe acute respiratory syndrome-related coronavirus
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 10 - DNA, 7950 bp, Artificial Sequence
  DNA equivalent of RNA replicon without antigen ORF
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggctgttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaataggg

SEQ ID NO: 11 - protein, 535 aa, Artificial Sequence
  nonstructural protein 1
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGA

SEQ ID NO: 12 - protein, 794 aa, Artificial Sequence
  nonstructural protein 2
GSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGC

SEQ ID NO: 13 - protein, 550 aa, Artificial Sequence
  nonstructural protein 3
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQ

SEQ ID NO: 14 - protein, 607 aa, Artificial Sequence
  nonstructural protein 4
YIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 15 - protein, 794 aa, Artificial Sequence
  nonstructural protein 2 with Q739L mutation
GSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARLFKFSRVCKPKSSLEETEVLFVFIGYDRKARTHNPYKLSSTLTNIYTGSRLHEAGC

SEQ ID NO: 16 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - DNA, 3822 bp, Artificial Sequence
  DNA equivalent of SARS-CoV-2 Spike protein
atgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacctga

SEQ ID NO: 18 - protein, 556 aa, Artificial Sequence
  nonstructural protein 4 variant after readthrough
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRFDAGA

SEQ ID NO: 19 - protein, 22 aa, Artificial Sequence
  Human tissue plasminogen activator leader peptide
MDAMKRGLCCVLLLCGAVFVSP

(19) United States
(12) Patent Application Publication
(10) Pub. No.: US 2023/0256083 A1
(43) Pub. Date: Aug. 17, 2023

(54) SELF-AMPLIFYING SARS-COV-2 RNA VACCINE

(71) Applicant: Ziphius Vaccines, Oostkamp (BE)

(72) Inventors: Itishri SAHU, Oostkamp (BE); Ashiqul HAQUE A K M, Oostkamp (BE); Sean MC CAFFERTY, Oostkamp (BE); Christiaan CARDON, Oostkamp (BE); Niek SANDERS, Merelbeke (BE)

(21) Appl. No.: 18/001,912

(22) PCT Filed: Jun. 18, 2021
(86) PCT No.: PCT/EP2021/066679

§ 371 Date: Dec. 15, 2022

(30) Foreign Application Priority Data
Jun. 19, 2020 (EP) 20181249.2
Jul. 3, 2020 (EP) 20184036.0
Apr. 23, 2021 (EP) 21170302.0

(51) Int. Cl. A61K 39/215; C07K 14/18; A61P 31/14

(52) U.S. Cl. CPC A61K 39/215; C07K 14/1808; A61P 31/14; A61K 2039/53

(57) ABSTRACT

The present invention relates self-replicating RNA molecules comprising a sequence encoding nonstructural alphavirus proteins and a sequence encoding a SARS-CoV-2 protein antigen.

### FIELD OF THE INVENTION

The present invention relates self-replicating RNA molecules comprising a sequence encoding nonstructural alphavirus proteins and a sequence encoding a SARS-CoV-2 protein antigen.

### BACKGROUND OF THE INVENTION

Coronaviruses (CoVs) are positive-sense single-stranded RNA viruses belonging to the family Coronaviridae (Ahmed at al., 2020). The newly emerging SARS-CoV-2 belongs to the betacoronaviruses, which are further divided into four lineages (that is, A-D). Lineage B, which includes SARS-CoV and SARS-CoV-2, has approximately 200 published virus sequences, whereas lineage C, which includes Middle East respiratory syndrome-related coronavirus (MERS-CoV), has over 500 viral sequences (Letko et al. 2020). SARS-CoV-2 has a genome size of ˜30 kilobases which, like other coronaviruses, encodes for multiple structural and non-structural proteins. The structural proteins include the spike (S) protein, the envelope (E) protein, the membrane (M) protein, and the nucleocapsid (N) protein. With SARS-CoV-2 being discovered very recently, there is currently a lack of information available about the virus. Preliminary studies suggest that SARS-CoV-2 is quite similar to SARS-CoV based on the full-length genome phylogenetic analysis, and the putatively similar cell entry mechanism and human cell receptor usage (Ahmed et al, 2020).

As SARS-CoV-2 is an RNA virus, it is rapidly evolving through mutations that are continuously arising in different territories worldwide. Therefore, mutations which are able to alter important viral properties, such as transmission rate or ability to cause disease, should be closely monitored. Recently, certain mutations have caused alterations of these important viral parameters, leading to three variants of concern, which are currently spreading globally (Volz et al., 2021). A first variant of concern, B1.1.7, originated in the UK and contains three key mutations in the gene encoding the Spike protein: N501Y, P681H and H69-V70. This variant has been associated with causing a higher grade in disease severity and infectiousness (Davies et al., 2021). A second variant of concern, B.1.351, was first identified in South Africa and contains three crucial substitution mutations in the Spike protein encoding region: N501Y, K417N and E484K. Data for this variant suggests that, due to the mutations, the virus has an increased rate of transmissibility and/or can more easily evade the immune response (Tegally et al., 2021). A last variant that is currently of concern, P.1, originated in Brazil and contains the same crucial mutations in the gene encoding the Spike protein, as does the South-African variant. Therefore, this variant also causes a higher transmission rate when compared to the wild-type virus (Moore & Offit, 2021). As the virus is constantly evolving, it is highly possible that additional SARS-CoV-2 variants will appear in the future.

All CoVs encode a surface glycoprotein, spike, which binds to the host-cell receptor and mediates viral entry. For betacoronaviruses, a single region of the spike protein called the receptor-binding domain (RBD) mediates the interaction with the host-cell receptor. After binding the receptor, a nearby host protease cleaves the spike, which releases the spike fusion peptide, facilitating virus entry. Known host receptors for betacoronaviruses include angiotensin-converting enzyme 2 (ACE2) for SARS-CoV and dipeptidyl peptidase-4 (DPP4) for MERS-CoV (Letko et al., 2020). Studies have shown that the SARS-CoV-2 spike binds ACE2 with higher affinity than SARS-CoV (Zhou et al., 2020). In addition, the nucleocapsid is also an important subunit for packaging the viral genome through protein oligomerization (Zhou et al., 2020). Protein sequence alignment analyses indicated that SARS-CoV-2 was most evolutionarily conserved with SARS-CoV. Specifically, the envelope and nucleocapsid proteins of SARS-CoV-2 are two evolutionarily conserved regions, with sequence identities of 96% and 89.6%, respectively, compared to SARS-CoV. However, the spike protein exhibited the lowest sequence conservation (sequence identity of 77%) between SARS-CoV-2 and SARS-CoV. Meanwhile, the spike protein of SARS-CoV-2 only has 31.9% sequence identity compared to MERS-CoV (Zhou et al., 2020).

In recent years, attention has turned to DNA (pDNA) vaccination. One of the main advantages of DNA vaccination compared to the more conventional vaccination methods is that the vaccine antigens are produced de novo by the host cells. DNA vaccines were able to induce potent T- and B-cell immune responses in animals against a variety of antigens. This culminated in the development and commercialization of plasmid DNA-based vaccines for animal usage (Davis et al., 2001; Garver et al., 2005; Kurath et al., 2006; Grosenbaugh et al., 2011). However, the development of DNA vaccines for humans has not been similarly successful thus far. While a number of clinical trials demonstrated the principle ability of DNA vaccines to induce cellular T- and B-cell responses in humans, the strength of these immune responses was rather lower than that achieved by more conventional approaches. Additionally, DNA vaccines have several drawbacks like e.g. the low efficacy in non-or slow dividing cells, the epigenetic silencing of the DNA construct, the presence of antibiotic resistance genes and a long-term uncontrolled expression, which may not necessarily correlate with good immune responses and may even be detrimental to the intended immune effect and lead to exhaustion of T cells (Wherry et al., 2003; Shin & Wherry, 2007; Han et al., 2010). A different explanation for the ‘underperformance’ of DNA vaccines in humans may be a weaker than presumed built in adjuvanticity of these vaccines. The importance of cytoplasmic DNA sensors for the induction of DNA-dependent immune responses has recently been increasingly recognized (Aoshi et al., 2011; Marichal et al., 2011; Desmet & Ishii, 2012). It is conceivable that without the assistance of sophisticated delivery methods, not enough DNA might end up in the cytoplasm or that species differences exist in the sensitivity of these sensors to stimulation by DNA (Kallen &Thef3, 2014). Therefore, researchers have turned to the application of RNA (mRNA) vaccines. The machinery of the transfected cell is utilized for in vivo translation of the message to the corresponding protein, which is the pharmacologically active product. The primary compartment of the pharmacodynamic activity of IVT mRNA is the cytoplasm. In contrast to natural mRNA that is produced in the nucleus and enters the cytoplasm through nuclear export, IVT mRNA has to enter the cytoplasm from the extracellular space. Once IVT mRNA has entered the cytoplasm, its pharmacology is governed by the same complex cellular mechanisms that regulate the stability and translation of native mRNA. The protein product translated from the IVT mRNA undergoes post-translational modification, and this protein is the bioactive compound. The half-lives of both the IVT mRNA template and the protein product are critical determinants of the pharmacokinetics of mRNA-based therapeutics. For immunotherapeutic approaches, the processing pathways of the encoded protein are crucial for determining its pharmacodynamics. Similar to the fate of endogenously generated protein, mRNA-encoded products are degraded by proteasomes and presented on major histocompatibility complex (MHC) class I molecules to CD8+ T cells. In general, intracellular proteins do not reach the MHC class II processing pathway to induce T helper cell responses. However, by introducing a secretion signal into the antigen-encoding sequence, T helper cell responses can be achieved as the secretion signal redirects the protein antigen to the extracellular space (Sahin et al., 2014).

In the 1990s, preclinical exploration of IVT mRNA was initiated for diverse applications, including protein substitution and vaccination approaches for cancer and infectious diseases. Consequently, accumulated knowledge enabled recent scientific and technological advances to overcome some of the obstacles associated with mRNA, such as its short half-life and unfavorable immunogenicity (Sahin et al., 2014).

After delivery of synthetic mRNAs in the cytosol of host cells, unmodified synthetic mRNAs are recognized as foreign and detected by pattern recognition receptors (PRRs), which leads to an innate immune response (Tam & Jacques, 2014). Synthetic mRNA can also activate the innate immune system by triggering the cytosolic RIG-I and MDA5 (Schlee et al., 2009; Goubau et al., 2014; Reikine et al., 2014). In addition, there are indications that synthetic mRNA molecules are also recognized by NLRs, leading to cell death via caspase-1 mediated pyroptosis (Bergsbaken et al., 2009; Andries et al., 2013). Therefore, genetic vaccines based on unmodified synthetic mRNAs can serve as excellent self-adjuvants by activating the expression of different cytokines that facilitate the cellular and humoral responses after mRNA vaccination. However, the innate immune response after carrier-mediated delivery of unmodified mRNA can be so vigorous that it leads to a blockage of mRNA translation and even mRNA degradation (Kormann et al., 2011; Katalin Kariko et al., 2012; Wu & Brewer, 2012; Zangi et al., 2013; Zhong et al., 2018). Moreover, recent reports showed that the elicited type I IFNs, resulting from the immune stimulatory properties of unmodified/non-HPLC purified synthetic mRNA, may negatively affect CD8+ T cell stimulation (De Beuckelaer et al., 2017). The inherent innate immunogenicity of synthetic mRNA is also a potential safety concern as type I interferons have been associated with thrombotic microangiopathy (Kavanagh et al., 2016), anemia (Libregts et al., 2011), and autoimmune diseases (Di Domizio & Cao, 2013). Consequently, it is important to seek for a perfect balance between mRNA translation and innate immune response. That being the case, the incorporation of naturally occurring modified nucleosides such as pseudouridine, 2-thiouridine, 5-methyluridine, 5-methylcytidine or N6-methyladenosine in IVT-mRNA has been shown to substantially reduce immune stimulation and stabilize the molecule against RNase cleavage (Katalin Karikó et al., 2005, 2012; B. R. Anderson et al., 2011; Kormann et al., 2011). Uridine depletion is the latest form of sequence engineering to reduce the immunogenecity and increase the translational activity (refer my paper: 10.1016/j.omtn.2018.06.010). Moreover, the immune stimulation of modified mRNA can be further reduced by purification with e.g. reversed-phase chromatography (Karikó et al., 2005; Bart R. Anderson et al., 2010; Andries, Mc Cafferty, et al., 2015). By modifying different structural elements of IVT-mRNA to systematically improve its intracellular stability and translational efficiency, a significant increase of protein expression from IVT-mRNA over several orders of magnitudes can be obtained (K Karikó et al., 1999; Holtkamp et al., 2006; Kallen & Theß, 2014).

For vaccination, the strong immune-stimulatory effect and intrinsic adjuvant activity of IVT mRNA lead to potent antigen-specific cellular and humoral immune responses (Sahin et al., 2014). The main advantage of using mRNA for vaccination is that the same molecule not only provides an antigen source for adaptive immunity, but can simultaneously bind to pattern recognition receptors, thus stimulating innate immunity. Humoral and cellular immune responses to protein antigens can be efficiently primed by nucleic acid or DNA/RNA vaccination. In nucleic acid-based vaccination, immunogenic proteins are expressed with correct posttranslational modification, conformation or oligomerization; this ensures the integrity of epitopes that stimulate neutralizing antibody (B cell) responses.

However, conceptually, there are several important differences between IVT mRNA-based approaches and other nucleic acid-based technologies, such as DNA vaccines. IVT mRNA does not need to enter into the nucleus to be functional; once it has reached the cytoplasm, the mRNA is translated instantly. By contrast, DNA need to access the nucleus to be transcribed into RNA, and their functionality depends on nuclear envelope breakdown during cell division (Sahin et al., 2014). Furthermore, mRNA can be delivered to non-dividing cells as it does not need to get into the nucleus as DNA plasmids (Sergeeva et al., 2016). In addition, IVT mRNA-based therapeutics, unlike plasmid DNA and viral vectors, do not integrate into the genome and therefore do not pose the risk of insertional mutagenesis. It is also advantageous that IVT mRNA is only transiently active and is completely degraded via physiological metabolic pathways (Sahin et al., 2014). RNA immunization is exceptionally potent in stimulating T cell responses because antigenic peptides are efficiently generated in (endogenous or exogenous) processing pathways (without interference by viral proteins) from intracellular or extracellular protein antigens expressed after transient in vivo transfection. Both features are difficult to achieve with recombinant subunit vaccines produced in eukaryotic or prokaryotic expression systems (Reimann & Schirmbeck, 2000). Although subunit vaccines produced from the natural infectious agent still fulfill an important role, the cost of producing and purifying immunogen can be prohibitive. By relying on the patient's body to make the desired protein, IVT mRNA drugs provide an approach in which the robust and tunable production of a therapeutic protein is possible, bypassing the need for costly manufacturing of proteins in fermentation tanks (Sahin et al., 2014) Additionally, roughly 10 years ago, Pascolo (2004), pointed out that the costs of manufacturing mRNA on a large scale would be lower than those to produce DNA (Pascolo, 2004). Nucleotide vaccines based on mRNA offer the flexibility to encode virtually any protein as antigen in a very short time span, but they could be produced with the same production process in the same production facilities. Thus novel vaccines could be made in a very short time with limited financial investments, which is of great importance for pandemic scenarios in infectious diseases (Kallen & Theß, 2014). The concept behind using IVT mRNA as a drug is the transfer of a defined genetic message into the cells of a patient for the ultimate purpose of preventing or altering a particular disease state (Sahin et al., 2014). In principle, two approaches of using IVT mRNA are being pursued. One is to transfer it into the patient's cells ex vivo; these transfected cells are then adoptively administered back to the patient. The second approach is direct delivery of the IVT mRNA in vivo using various routes. Self-amplifying (sa) viral mRNA replicons harbor the RDRP genes and mimic the characteristic replicative features of positive-strand RNA viruses (Etchinson & Ehrenfeld, 1981; Mizutani & Colonno, 1985; van der Werf et al., 1986; C. M. Rice et al., 1987; Liljestrom & Garoff, 1991; Rolls et al., 1994; Khromykh & Westaway, 1997; Perri et al., 2003). The replicon RNA can be produced easily by in vitro transcription from cDNA templates. The structural genes of the RNA virus are replaced by heterologous genes of interest, which are controlled by a subgenomic promoter (Xiong et al., 1989; Zhou et al., 1994; Ying et al., 1999; Hewson, 2000; Lundstrom, 2009).

Recently, sa-mRNA (RNA replicon) vaccination is recognized as innovative nanotechnology-based vaccination strategy (Andries, Kitada, et al., 2015). As mentioned previously, unlike viral replicon particles (i.e. RNA encapsulated in viral capsid proteins), RNA replicon can be produced by in vitro transcription only. Thus, the whole manufacturing process is entirely cell-free, resulting in a therapeutic whose composition is precisely defined. RNA replicon vaccines have several attractive features, such as extending the duration (approximately 2 months) and magnitude of expression compared to their non-replicating counterparts (Kowalski et al., 2019). In addition, the intracellular replication of sa-mRNA is transient and it produces double-stranded RNA (dsRNA) intermediate during replication which can induce interferon-mediated host-defense mechanisms by triggering pattern recognition receptors. This results in strong antigen-specific immune responses against the inserted target molecules. Thus, sa-mRNA vector systems are ideally suited for vaccine development because they provide high transient transgene expression and inherent adjuvant effects (Sahin et al., 2014).

According to the common classification, two types of vectors can be used to deliver genetic material to the target cells. On one hand, viral vectors, that mimic the behavior of their precursor viruses, are used. Different types of vectors, including retroviral, lentiviral, adenoviral, and adeno-associated viruses have been employed and are even being approved for clinical use in Europe. On the other hand there are non-viral vectors that can be described according to their composition. The most commonly cited are lipoplexes (lipid+DNA or RNA) and polyplexes (polymer+DNA or RNA) (Perez Ruiz de Garibay, 2016).

Performing gene delivery with deactivated or non-replicating viral vectors comprises about two-thirds of clinical trials (Ginn et al., 2018), with the selection of any particular viral vector depending on the therapeutic target. For example, the frequently employed adenovirus serotype 5 (Ad5) vectors can target either dividing or non-dividing cells. Although Ad5 viruses are stable and easy to manipulate genetically, their immunogenicity hinders clinical translation (Salameh et al., 2019). Adeno-associated virus (AAV) is one of the most actively investigated gene therapy vehicles and has in general been shown to be less immunogenic than other viruses. However, pre-existing immunity to AAV, especially the presence of circulating neutralizing antibodies, can have a dramatic effect on AAV clinical efficacy. To date, this represents one of the biggest therapeutic challenges to the use of systemically delivered AAV, and is thought to be one of the factors in early clinical failures (Naso et al., 2017). Retroviruses and lentiviruses can integrate their genome into host cells, resulting in long-term transgene expression. However, retroviruses can only transfect actively dividing cells, and thus are precluded from targeting non-dividing cells (e.g., in brain tissue). Moreover, retroviral and lentiviral vectors are costly to manufacture and less stable as recombinant vectors than Ad5, hindering reproducibility of gene transfer. Innate and adaptive immune responses induced by viral vectors further limit their efficacy. Thus, many efforts focus on masking immunogenicity by covalent attachment of synthetic polymers, such as poly(ethylene glycol) (PEG) and poly-N-(2-hydroxypropyl) methacrylamide poly(HPMA). Such polymer-virus hybrids can generate stable, sustained gene expression, and transfect non-dividing cells (Ramsey et al., 2010). Unfortunately, the reduction in side-effects coincides with an undesirably large reduction in transfection efficiency (Salameh et al., 2019).

On the contrary, non-viral vectors prepared from polymers, liposomes, or other nanoscale structures offer routes to overcome the drawbacks of viral vectors. Non-viral vectors are safer platforms, and their production is simpler, cheaper and more reproducible than viral vectors. Moreover, there is no limitation in the cargo of DNA or RNA that can be delivered. Efficacy of transfection of non-viral vectors is their major limitation, although it has been improved by several strategies, resulting in an increased number of products entering into clinical trials (del Pozo-Rodriguez et al., 2016; Molla & Levkin, 2016; Perez Ruiz de Garibay, 2016).

Nucleic acids that encode gene products, such as proteins and RNA (e.g., small RNA) can be delivered directly to a desired vertebrate subject or can be delivered ex vivo to cells obtained or derived from the subject, and the cells can be re-implanted into the subject. Delivery of such nucleic acids to a vertebrate subject is desirable for many purposes, such as, for gene therapy, to induce an immune response against an encoded polypeptide, or to regulate the expression of endogenous genes. The use of this approach has been hindered because free DNA is not readily taken up by cells, and free RNA is rapidly degraded in vivo. Accordingly, nucleic acid delivery systems have been used to improve the efficiency of nucleic acid delivery.

Nucleic acid delivery systems can be classified into two general categories, recombinant viral system and nonviral systems. Viruses, as viral vectors, are highly efficient delivery systems that have evolved to infect cells. Some viruses have been altered to produce viral vectors that are not infectious, but are still able to efficiently deliver nucleic acids that encode exogenous gene products to host cells. However, certain types of virus vectors, such as recombinant viruses, still have potential safety and effectiveness concerns. For example, infectious virus may be produced through recombination events between vector components when a vector is produced using a method that involves packaging, viral proteins may induce an undesirable immune response, which can shorten the time of transgene expression and even prevent repetitive use of the recombinant virus. See, e.g., Seung et al. Gene Therapy 10:706-711 (2003), Tsai et al. Clin. Cancer. Res. 10:7199-7206 (2004).

In addition, there are limitations on the size of the nucleic acid that can be delivered using recombinant viruses, which can prevent the delivery of large nucleic acids or multiple nucleic acids. Commonly investigated non-viral delivery systems include delivery of free nucleic acid such as DNA or RNA, and delivery of formulations that contain nucleic acid and lipids (e.g., liposomes), polycations or other agents intended to increase the rate of transfection. See, e.g., Montana et al, Bioconjugate Chem. 18:302-308 (2007), Ouahabi et al., FEBS Letters, 380:108-112, (1996).

However, these types of delivery systems are generally less efficient than recombinant viruses.

The immune response induced by nucleic acid vaccines should include reactivity to the antigen encoded by the nucleic acid and confer pathogen-specific immunity. Antigen duration, dose and the type of antigen presentation to the immune system are important factors that relate to the type and magnitude of an immune response. The efficacy of nucleic acid vaccination is often limited by inefficient uptake of the nucleic acids into cells. Generally, less than 1% of the muscle or skin cells at the site of injection express the gene of interest. This low efficiency is particularly problematic when it is desirable for the genetic vaccine to enter a particular subset of the cells present in a target tissue. See, e.g., Restifo et al., Gene Therapy 7:89-92 (2000).

Self-replicating RNA molecules, which replicate in host cells leading to an amplification of the amount of RNA encoding the desired gene product, can enhance efficiency of RNA delivery and expression of the encoded gene products. See, e.g., Johanning, F. W., et al, Nucleic Acids Res., 23(9):1495-1501 (1995); Khromykh, A. A., Current Opinion in Molecular Therapeutics, 2(5):556-570 (2000); Smerdou et al., Current Opinion in Molecular Therapeutics, 1(2):244-251 (1999). Self-replicating RNAs have been produced as virus particles and as free RNA molecules. However, free RNA molecules are rapidly degraded in vivo, and most RNA-based vaccines that have been tested have had limited ability to provide antigen at a dose and duration required to produce a strong, durable immune response. See, e.g., Probst et al., Genetic Vaccines and Therapy, 4:4; doi:10.1186/1479-0556-4-4 (2006).

COVID-19, the infectious disease caused by SARS-CoV-2 resulted in a global pandemic. Currently, more than 175 million cases have been reported across 188 countries and territories, resulting in about 3,700,00 deaths and a flooding of emergency room capacity. The outbreak has also been proven a threat to the global economy, making it the costliest disaster ever in human history. Despite the drastic measures taken by governments throughout the world and despite some vaccines being approved through emergency procedures, there remains an absolute urgency and need in obtaining an effective SARS-CoV-2 vaccine capable of iliciting sustained protection against various SARS-CoV-2 variants. It has been proven that vaccine development for these viruses is a daunting task as several reports question the longevity of antibody responses in SARS-CoV-2 infected patients and homologues re-infections have been shown. Furthermore, patients, even those with only mild symptoms, may presents symptoms for very long durations, indicating viral persistency. An additional concern relates to the rapid evolution of the virus and the apparition of SARS-CoV-2 variants having altered viral properties, including transmission rate and ability to cause disease. Finally, recovered patients without detectable SARS-CoV-2 have been shown to test positive at later timeframes, demonstrating the occurrence of persistent or recurrent infections. Thus, there is a need for an effective SARS-CoV-2 vaccine that generates a sufficient immune response to significantly reduce the chance of SARS-CoV-2 infections and severe disease and preferably does so with long-term efficacy, and at the same time allows for robust protection against known and future SARS-CoV-2 variants.

Sheahan et al. (2011) describes immunization of mice with a Venezuelan equine encephalitis virus replicon particle vaccine comprising the S protein of SARS-CoV as antigen. The technology was however not optimized for human use, nor did it disclose any effect against SARS-CoV-2.

While meanwhile two RNA vaccines for SARS-CoV-2 have been approved by EMA and the FDA through a Conditional Marketing Authorisation procedure, specific for emergencies, these vaccines require a high multi-dose of mRNA. The production of these vaccines is expensive, and time-consuming. In addition, high concentrations of RNA could cause side-effects in the patients receiving a dose. Hence, there is a need for a more efficient and more potent RNA vaccine that solves one or more of the problems listed herein.

### SUMMARY OF THE INVENTION

The inventors have now surprisingly identified self-replicating RNA molecules comprising a SARS-CoV-2 antigen, as detailed in the claims, which fulfils the above-mentioned needs. In particular, it has been found that the combination of a sequence encoding a SARS-CoV-2 Spike protein antigen and a sequence encoding a SARS-CoV-2 Nucleocapsid antigen leads to strong binding antibody response, efficient neutralization of SARS-CoV-2 in vivo, elicitation of both CD4+ and CD8+ T cell immunity, and provides robust protection against various SARS-CoV-2 variants.

In a first aspect, the present invention provides a self-replicating RNA molecule comprising a sequence encoding a SARS-CoV-2 antigen. In some embodiments, the self-replicating RNA molecules are based on the RNA genome of an alpha virus/a Venezuelan Equine Encephalitis virus. Preferably, the self-replicating RNA molecules contain a heterologous sequence encoding a gene product, such as a target protein (e.g. an antigen) or an RNA (e.g., a small RNA)/at least one SARS-CoV-2 antigen. In a particular embodiment, the self-replicating RNA molecule contains a mutation, preferably both an A3G substitution in the 5′UTR and an nsP2 Q739L mutation. In a particular embodiment, the SARS-CoV-2 antigen is a SARS-CoV-2 Spike protein antigen. In a further embodiment, the Spike protein antigen is a truncated form of the Spike protein comprising the Receptor-Binding Domain (RBD). In a further embodiment, the Spike protein is fused to an immune stimulating protein, such as C3d-p28.

In another particular embodiment, the SARS-CoV-2 antigen is a SARS-CoV-2 Nucleocapsid protein antigen. In yet another particular embodiment, the SARS-CoV-2 antigen is a SARS-CoV-2 Membrane protein antigen. In a preferred embodiment, the self-replicating RNA molecule comprises a sequence encoding a SARS-CoV-2 Spike protein antigen and further comprises a sequence encoding a SARS-CoV-2 Nucleocapsid protein antigen and/or a SARS-Cov-2 Membrane protein antigen. In one embodiment, the present invention provides a self-replicating RNA molecule comprising a sequence encoding a SARS-CoV-2 Spike protein antigen and further comprising a sequence encoding a SARS-CoV-2 Nucleocapsid protein antigen. In one other embodiment, the present invention provides a self-replicating RNA molecule comprising a sequence encoding a SARS-CoV-2 Spike protein antigen and further comprising a sequence encoding a SARS-CoV-2 Membrane protein antigen. In another aspect, the invention relates to pharmaceutical compositions (e.g; immunogenic compositions and vaccines) that comprise a self-replicating RNA molecule as described herein, and a pharmaceutically acceptable carrier and/or a pharmaceutically acceptable vehicle. The pharmaceutical composition can further comprise at least one adjuvant and/or a nucleic acid delivery system. In some embodiments, the composition further comprises a cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, or a polycationic peptide, a cationic nano-emulsion or combinations thereof.

In particular embodiments, the self-replicating RNA molecule is encapsulated in, bound to or adsorbed on a cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, a cationic nano-emulsion or combinations thereof.

In another aspect, the invention relates to methods of using the self-replicating RNA molecules and pharmaceutical compositions described herein, including medical use to treat or prevent disease, such as an infectious disease, particularly a coronaviral disease. In a particular embodiment, the present invention provide the self-replicating RNA molecules and compositions as described herein for the prevention and/or treatment of a disease caused by a SARS-coronavirus, particularly a disease caused by SARS-CoV-2, more in particular COVID-19. Thus, the present invention also provide the self-replicating RNA molecules and compositions as described herein for vaccinating against SARS-CoV-2. Such methods comprise administering an effective amount of a self-replicating RNA molecule or composition as described herein, to a subject in need thereof. For example, the invention provides for the use of self-replicating RNA molecules of the invention that encode an antigen for inducing an immune response in a subject.

The present invention also provides methods for inducing production of a SARS-CoV-2 antigen in a subject, the method comprising administering an effective amount of a self-replicating RNA molecule or composition of the invention to said subject. The invention also relates to a method for inducing an immune response in a subject comprising administering to the subject an effective amount of a pharmaceutical composition as described herein. In a further embodiment, the present invention also provides methods for inducing production of anti-SARS-CoV-2 antibodies in a subject, the method comprising administering an effective amount of a self-replicating RNA molecule or composition of the invention to said subject.

The invention also relates to a method of vaccinating a subject, comprising administering to the subject a pharmaceutical composition as described herein.

The invention also relates to a method for inducing a mammalian cell to produce a SARS-CoV-2 and antigen, comprising the step of contacting the cell with a pharmaceutical composition as described herein, under conditions suitable for the uptake of the self-replicating RNA molecule by the cell.

The invention also relates to a method for gene delivery comprising administering to a pharmaceutical composition as described herein.

In particular, the invention is defined in the following embodiments, which are not limitative to the invention:

1. A combination comprising a sequence encoding a SARS-CoV-2 Spike protein antigen and a sequence encoding a SARS-CoV-2 Nucleocapsid antigen, wherein the sequence encoding the SARS-CoV-2 Spike protein antigen and the sequence encoding the SARS-CoV-2 Nucleocapsid protein antigen are comprised in one or more self-replicating RNA molecules and wherein the one or more self-replicating RNA molecules further comprise a sequence encoding nonstructural alphavirus proteins.

2. The combination of embodiment 1, wherein the sequence encoding the SARS-CoV-2 Spike protein antigen and the sequence encoding the SARS-CoV-2 Nucleocapsid protein antigen are comprised in the same self-replicating RNA molecule, or wherein the sequence encoding the SARS-CoV-2 Spike protein antigen and the sequence encoding the SARS-CoV-2 Nucleocapsid protein antigen are comprised in different self-replicating RNA molecules.

3. The combination of embodiment 1 or 2, wherein the alphavirus is a Venezuelan Equine Encephalitis Virus (VEEV), such as strain TC-83 or a strain having at least 90% sequence identity, preferably at least 95%, more preferably at least 97% thereto.

4. The combination of any one of the previous embodiments, wherein the one or more self-replicating RNA molecules comprise an A3G mutation in the 5′UTR and/or a Q739L mutation in Nonstructural Protein 2 (nsP2).

5. The combination of any one of the previous embodiments, wherein the Spike protein antigen is a truncated form of the Spike protein comprising the Receptor-Binding Domain (RBD).

6. The combination of embodiment 5, wherein the RBD corresponds to SEQ ID NO: 1 or an amino acid sequence having at least 95% identity, preferably at least 97% sequence identity, more preferably at least 99% sequence identity thereto.

7. The combination of any of the previous embodiments, wherein the one or more self-replicating RNA molecules comprise the nonstructural proteins of VEEV strain TC-83, an A3G mutation in the 5′UTR and a Q739L mutation in nsP2.

8. The combination of any of the previous embodiments wherein said sequence encoding the SARS-CoV-2 Spike protein antigen comprises a 5′ cap, followed by a sequence encoding nonstructural alphavirus proteins nsP1, nsP2, nsP3 and nsP4, a subgenomic promoter followed by a sequence encoding for the SARS-CoV-2 Spike protein antigen or a truncated form of the Spike protein comprising the Receptor-Binding Domain (RBD) and a poly-A tail downstream of said SARS-CoV-2 Spike protein antigen or truncated form.

9. The combination of any of the previous embodiments wherein said sequence encoding a SARS-CoV-2 Nucleocapsid (N) antigen comprises a 5′ cap, followed by a sequence encoding nonstructural alphavirus proteins nsP1, nsP2, nsP3 and nsP4, a subgenomic promoter followed by a sequence encoding for the SARS-CoV-2 N protein antigen and a poly-A tail downstream of said SARS-CoV-2 N protein antigen.

10. A pharmaceutical composition comprising the combination according to any one of the previous embodiments and a pharmaceutically acceptable carrier and/or a pharmaceutically acceptable vehicle.

11. The pharmaceutical composition of embodiment 10, further comprising at least one adjuvant.

12. The pharmaceutical composition of embodiment 10 or 11, further comprising a cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, and a polycationic peptide, or a cationic nano-emulsion.

13. The pharmaceutical composition of any one of embodiments 10 to 12, wherein the one or more self-replicating RNA molecules are encapsulated in, bound to or adsorbed on a cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, a cationic nano-emulsion and combinations thereof.

14. A vaccine, comprising a combination according to any of the previous embodiments wherein said RNA molecules are encapsulated in, bound to or adsorbed on a cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, a cationic nano-emulsion and combinations thereof and wherein the effective dose of said RNA in said vaccine is between 0.1 and 100 μg.

15. The combination of any one of embodiments 1 to 9 or the pharmaceutical composition of any one of embodiments 10 to 13 or the vaccine according to embodiment 14, for use as a medicine.

16. The combination of any one of embodiments 1 to 9 or the pharmaceutical composition of any one of embodiments 10 to 13 or the vaccine according to embodiment 14, for use in the prevention and/or treatment of an infectious disease.

17. The combination of any one of embodiments 1 to 9 or the pharmaceutical composition of any one of embodiments 10 to 13 or the vaccine for use according to embodiment 14, for use in inducing an immune response in a subject.

18. The combination of any one of embodiments 1 to 9 or the pharmaceutical composition of any one of embodiments 10 to 13 or vaccine according to embodiment 14, for use in vaccinating a subject against a coronaviral disease, such as SARS-CoV, SARS-CoV-2 or MERS-CoV.

19. The combination, the pharmaceutical composition or the vaccine for use according to any of the embodiments 15 to 18 wherein an effective dose of said RNA is between 0.1 and 100 μg.

20. The combination, the pharmaceutical composition or the vaccine for use according to any of the embodiments 15 to 19, wherein said combination, composition or vaccine is administered intramuscular, intradermal or subcutaneous.

21. The combination, the pharmaceutical composition or the vaccine for use according to any of the embodiments 15 to 20, wherein said combination, composition or vaccine is administered as a single dose or as a multi-dose, requiring a series of two or more doses, administered within a pre-defined timespan.

22. The combination, the pharmaceutical composition or the vaccine for use according to any of the embodiments 15 to 21, wherein said combination, composition or vaccine is administered periodically, such as annually or bi-annually.

23. The combination, the pharmaceutical composition or the vaccine for use according to any of the previous embodiments, wherein a dose of said vaccine is between 0.05 and 1 ml.

24. A coronavirus vaccine comprising self-replicating RNA molecules, said each self replicating RNA molecule comprises a sequence encoding nonstructural alphavirus proteins and a sequence encoding a SARS-CoV-2 Spike protein antigen, wherein said RNA molecules are encapsulated in, bound to or adsorbed on a cationic lipid, a lipid nanoparticle, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, a cationic nano-emulsion and combinations thereof.

25. The vaccine of embodiment 24, wherein the alphavirus is a Venezuelan Equine Encephalitis Virus (VEEV), such as strain TC-83 or a strain having at least 90% sequence identity, preferably at least 95%, more preferably at least 97% thereto.

26. The vaccine of embodiment 24 or 25 comprising an A3G mutation in the 5′UTR and/or a Q739L mutation in Nonstructural Protein 2 (nsP2).

27. The vaccine of any one of the previous embodiments 24 to 26, wherein the Spike protein antigen is a truncated form of the Spike protein comprising the Receptor-Binding Domain (RBD), wherein the RBD corresponds to SEQ ID NO: 1 or an amino acid sequence having at least 95% identity, preferably at least 97% sequence identity, more preferably at least 99% sequence identity thereto.

28. The vaccine of any one of the previous embodiments 24 to 27, wherein the Spike protein is fused to an immune stimulating protein, such as C3d-p28.

29. The vaccine of any one of the previous embodiments 24 to 28, further comprising a sequence encoding a SARS-CoV-2 Nucleocapsid protein antigen and/or a SARS-Cov-2 Membrane protein antigen.

30. The vaccine of any one of the previous embodiments 24 to 29, comprising the nonstructural proteins of VEEV strain TC-83, an A3G mutation in the 5′UTR and a Q739L mutation in nsP2, and a 25 sequence encoding a truncated form of the SARS-CoV-2 Spike protein comprising the RBD.

31. The vaccine of any of the previous embodiments, wherein said each self-amplifying RNA molecule comprises a 5′ cap, followed by a sequence encoding nonstructural alphavirus proteins nsP1, nsP2, nsP3 and nsP4, a subgenomic promoter followed by a sequence encoding for the SARS-CoV-2 Spike protein antigen or a truncated form of the Spike protein comprising the Receptor-Binding Domain (RBD) and a poly-A tail downstream of said SARS-CoV-2 Spike protein antigen or truncated form.

32. The vaccine of any of the previous embodiments 24 to 31, wherein said vaccine, when administered to a subject, preferably a human subject, is able to elicit or induce an immune response to SARS-COV-2 and/or variants thereof in said subject.

33. The vaccine according to any of the previous embodiments 24 to 32, wherein the vaccine is formulated such that one dose of said vaccine comprises between 0.1 μg and 100 μg of RNA.

34. The vaccine according to any of the previous embodiments 24 to 33, wherein a dose of said vaccine is between 0.05 and 1 ml.

35. The vaccine according to any of the previous embodiments, further comprising an adjuvant.

36. A method of treatment or prophylaxis of coronavirus infection, preferably SARS-CoV-2, said method comprises administering a combination according to any of the embodiments 1 to 9, a pharmaceutical composition according or a vaccine according to any of the embodiments 10 to 13 or 14 to 35 to a subject, preferably a human subject.

37. A method of inducing an immuneresponse in a subject against a coronavirus infection, preferably SARS-CoV-2, said method comprises administering a combination according to any of the embodiments 1 to 9, a pharmaceutical composition according or a vaccine according to any of the embodiments 10 to 13 or 14 to 35 to a subject, preferably a human subject.

38. The method according to embodiment 36 or 37, wherein said combination, pharmaceutical composition or vaccine is administered to said subject by subcutaneous, intramuscular or intradermal injection.

39. The method according to any of the previous embodiments wherein an administered dose comprises between 0.1 and 100 μg RNA.

40. Method according to any of the previous embodiments wherein said combination, pharmaceutical composition or vaccine is administered as a single dose or as a multi-dose, requiring a series of two or more doses, administered within a pre-defined timespan.

41. Method according to any of the previous embodiments, wherein said combination, composition or vaccine is administered periodically, such as annually or bi-annually

42. A vector, said vector comprises:
an antigen sequence, wherein said antigen sequence encodes for an antigen of SARS-CoV-2 and wherein said antigen is downstream of a promoter sequence, preferably an alphavirus derived subgenomic promoter (SGP);
a poly(A) sequence downstream of said antigen sequence; and
sequences encoding for non-structural proteins nsP1 to 4 of the Venezuelan Equine

Encephalitis Virus.

43. The vector according to embodiment 42, wherein said antigen sequence encodes for a SARS-CoV-2 Spike protein or a truncated form thereof, or wherein said antigen sequence encodes for a SARS-CoV-2 Nucleocapsid (N) antigen.

44. The vector according to embodiments 42 and 43, wherein said truncated form of the Spike protein comprising the Receptor-Binding Domain (RBD).

45. The vector according to any of the previous embodiments, wherein the sequence of nsP2 is such that it encodes for an nsP2 protein having a Q739L mutation and/or an A3G mutation in the 5′UTR of said vector.

46. The vector according to any of the previous embodiments, wherein said vector is a plasmid or linearized DNA.

### BRIEF DESCRIPTION OF FIGURES

FIG. 1 Western Blot indicating the saRNA's ability of expression of S1-RBD protein expression.

FIG. 2 Western Blot indicating the saRNA's ability of expression of N protein expression.

FIG. 3 Immunogenicity of saRNA-S1 RBD after intradermal injection+electroporation.

FIG. 4 Immunogenicity of saRNA-S1 RBD intramuscular injection FIG. 5 shows antigen expression in SWISS mice following in vivo administration of a mock vaccine (luciferase)

FIG. 6 shows induction of SARS-CoV-2 S-specific binding antibody response induction following in vivo S-RBD and S-RBD+N prime vaccination of SWISS mice. Data is presented as individual values (n=6 mice per group) with mean±SD; ns=non-significant, *p<0.05, **p>0.01, ***p>0.001, ****p<0.0001.

FIG. 7 shows induction of SARS-CoV-2 N-specific binding antibody response induction following in vivo N and S-RBD+N prime vaccination of SWISS mice. Data is presented as individual values (n=6 mice per group) with mean±SD; ns=non-significant, *p<0.05, **p>0.01, ***p>0.001, ****p<0.0001.

FIG. 8 shows induction of SARS-CoV-2 S-specific binding antibody response induction following in vivo S-RBD and S-RBD+N prime vaccination of SWISS mice, which is enhanced by booster immunization. Data is presented as individual values (n=6 mice per group) with mean±SD; ns=non-significant, *p<0.05, **p>0.01, ***p>0.001, ****p<0.0001.

FIG. 9 shows induction of SARS-CoV-2 N-specific binding antibody response induction following in vivo N and S-RBD+N prime vaccination of SWISS mice, which is enhanced by booster immunization. Data is presented as individual values (n=6 mice per group) with mean±SD; ns=non-significant, *p<0.05, **p>0.01, ***p>0.001, ****p<0.0001.

FIG. 10 shows that vaccination with sa-RNA-S-RBD and the combination sa-RNA-S-RBD+sa-RNA-N induces neutralizing antibodies against wild-type SARS-Co-2 (Wuhan) in mice.

FIG. 11 shows weight change of hamsters between day of infection with wild-type SARS-Co-2 (Wuhan) and day of sacrifice.

FIG. 12 shows the amount of SARS-CoV-2 RNA genome copies per mg lung tissue in hamsters.

FIG. 13 shows the amount of virus required to produce a cytopathic effect in 50% of cells inoculated with serum from hamsters (TCID50).

FIG. 14 shows that immunization with LNP S-RBD+N saRNA induces neutralizing antibodies (Wuhan SARS-CoV strain) in hamsters.

FIG. 15 shows that low doses of ZIP-LNP S-RBD+N saRNA (ZIP1642) immunization reduces IL-6 cytokine mRNA expression in lung tissue of vaccinated hamsters.

FIG. 16 show that ZIP-LNP S-RBD+N saRNA (ZIP1642) immunization reduces IP-chemokine mRNA expression in lung tissue of vaccinated hamsters.

FIG. 17 shows that ZIP-LNP S-RBD+N saRNA (ZIP1642) immunization at 1 and 5 μg dosage reduces histopathology features of the lung of SARS-CoV-2 infected hamsters.

FIG. 18 shows that ZIP-LNP S-RBD+N saRNA (ZIP1642) immunization at 1 and 5 μg dosage induces robust SARS-CoV-2 specific binding antibody responses in hamsters

FIG. 19 shows an example of a S-RBD+N saRNA construct that can be used as RNA component of a vaccine as described herein.

FIG. 20 shows the S-specific T cell response of Th2, Th1 and CTL cells following in vivo S-RBD and S-RBD+N prime vaccination of SWISS mice. Data is presented as individual values (n=6 mice per group) with mean±SD; ns=non-significant, *p<0.05, **p>0.01, ***p>0.001, ****p<0.0001.

### DETAILED DESCRIPTION OF THE INVENTION

The present invention relates to self-replicating RNA molecules and methods for using self-replicating RNA for therapeutic purposes, such as for immunization or gene therapy. In particular, the present invention provides method for the active immunization to prevent COVID-19 caused by SARS-CoV-2 virus.

Recently, sa-mRNA (RNA replicon) vaccination is recognized as innovative nanotechnology-based vaccination strategy (Andries, Kitada, et al., 2015). Unlike viral replicon particles (i.e. RNA encapsulated in viral capsid proteins), RNA replicon can be produced by in vitro transcription only. Thus, the whole manufacturing process is entirely cell-free, resulting in a therapeutic whose composition is precisely defined. RNA replicon vaccines have several attractive features, such as extending the duration (approximately 2 months) and magnitude of expression compared to their non-replicating counterparts (Kowalski et al., 2019). In addition, the intracellular replication of sa-mRNA is transient and the double-stranded RNA (dsRNA) induces interferon-mediated host-defense mechanisms by triggering pattern recognition receptors. This results in strong antigen-specific immune responses against the inserted target molecules. Thus, sa-mRNA vector systems are ideally suited for vaccine development because they provide high transient transgene expression and inherent adjuvant effects (Sahin et al., 2014).

Self-replicating RNA molecules as described herein (e.g., when delivered in the form of naked RNA) can amplify themselves and initiate expression and overexpression of heterologous gene products in the host cell. Self-replicating RNA molecules of the invention, unlike mRNA, use their own encoded viral polymerase to amplify itself. The self-replicating RNA molecules of the invention, such as those based on alphaviruses, generate large amounts of sub-genomic mRNAs from which large amounts of proteins (or small RNAs) can be expressed. Self-replicating RNA molecules are herein also referred to as “replicons”.

### Definitions

“Nucleotide” is a term of art that refers to a molecule that contains a nucleoside or deoxynucleoside, and at least one phosphate. A nucleoside or deoxynucleoside contains a single 5 carbon sugar moiety (e.g., ribose or deoxyribose) linked to a nitrogenous base, which is either a substituted pyrimidine (e.g., cytosine (C), thymine (T) or uracil (U)) or a substituted purine (e.g., adenine (A) or guanine (G)). As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. RNA sequences may be presented herein using their “DNA equivalent” sequences. As is well-known, a DNA equivalent sequence can be readily converted to the RNA sequence that it represents by replacing thymine (T) with uracil (U).

“Sequence identity”. Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux et al., Nucleic Acids Research 12(1): 387, 1984), BLASTP, BLASTN, and FASTA (Altschul et al. 3. Mol. Biol. 215: 403-410, 1990). The BLAST X program is publicly available from NCBI and other sources (BLAST Manual Altschul et al. NCBI NLM NIH Bethesda, Md. 20894; Altschul et al. 3. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An “effective amount” of a self-replicating RNA refers to an amount sufficient to elicit expression of a detectable amount of a SARS-CoV-2 antigen, particularly to induce a SARS-CoV-2 antigen-specific response, preferably an amount suitable to produce a desired therapeutic or prophylactic effect.

The term “naked” as used herein refers to nucleic acids that are substantially free of other macromolecules, such as lipids, polymers, and proteins. A “naked” nucleic acid, such as a self-replicating RNA, is not formulated with other macromolecules to improve cellular uptake. Accordingly, a naked nucleic acid is not encapsulated in, absorbed on, or bound to a liposome, a microparticle or nanoparticle, a cationic emulsion, and the like.

The terms “treat,” “treating” or “treatment”, as used herein, include alleviating, abating or ameliorating disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the term “coronaviral disease” refers to a disease associated with an infection with a coronavirus, particularly a disease caused by the infection with a coronavirus.

As used herein, the term “coronavirus” includes any member of the family Coronaviridae, including, but not limited to, the subfamilies Letovirinae and Orthocoronavirinae. They thus include the genera Alpha coronavirus, Betacoronavirus, Gammacoronavirus and Delta corona virus. Preferred coronaviruses in the context of the invention are the betacoronaviruses, particularly Severe acute respiratory syndrome-related coronaviruses (including SARS-CoV-1 and SARS-CoV-2). SARS-CoV-2 has various known mutations called variants, whereby a variant may pertain to a collection of similar characteristical mutations (referred to as a lineage). A first variant, B1.1.7, originated in the UK and contains three key mutations in the gene encoding the Spike protein: N501Y, P681H and H69-V70. A second variant, B.1.351, was first identified in South Africa and contains three crucial substitution mutations in the Spike protein encoding region: N501Y, K417N and E484K. A third variant, P.1, originated in Brazil and contains the same crucial mutations in the gene encoding the Spike protein, as does the South-African variant. A fourth variant, B.1.617, was first identified in India and contains three crucial substitution mutations in the Spike protein encoding region: E484Q, L452R, P681R. The term SARS-CoV-2 as used herein encompasses all variants that are to date known, unless the variant is specifically mentioned.

As used herein, the term “immune response” pertains to the reaction within the body that is caused by antigens (=foreign substances) and results in the production of antibodies that can fight disease by killing or inhibiting the causative agent.

As used herein, the term “alphaviruses” comprise a genus (Alphavirus) in the family Togaviridae of enveloped, single-stranded, positive-sense RNA viruses that occur nearly worldwide. Alphaviruses are zoonotic pathogens that are maintained primarily in rodents, primates, and birds by mosquito vectors, although a few infect fish and seals and may have no arthropod vector. Human disease occurs when people intrude on enzootic transmission habitats and are bitten by infected mosquitoes, or when alphaviruses emerge to cause epizootics and epidemics.

As used herein, the term subgenomic promoter refers to those sequences that constitute a functional element required for production of subgenomic RNA species. A subgenomic promoter is necessary to drive expression of genes using RNA as template; the subgenomic promoter is recognized by an RNA-dependent RNA polymerase, which may be a viral RNA replicase. The promoter itself may be a composite of segments derived from more than one source, naturally occurring or synthetic. It should be noted that a subgenomic promoter is located in relation to a subgenomic RNA species or a particular gene whose transcription it initiates, and it is functionally recognized by an RNA-dependent RNA polymerase (or viral RNA replicase) when it is contained within an RNA molecule of the proper (−) polarity. The (−) sense RNA molecule containing the functional copy of the subgenomic promoter, which comprises the functional units of core promoter and activating domain, can be synthesized by RNA-dependent RNA polymerase using a (+) sense RNA molecule as template, or it may have been synthesized by (cellular) RNA polymerase II as a transcript initiated by a polII promoter.

According to the invention, a vaccine may have at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs). 5′-capping of modified RNA may be completed post-transcriptionally using a Vaccinia Virus Capping Enzyme to generate the “Cap 0” structure: m7G(5′)ppp(5′)G (New England BioLabscap). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source. Alternatively, 5′-capping of polynucleotides may be achieved by co-transcriptional capping using m2 7,3′-OGpppG or m2 7,2′-OGpppG ARCA (CellScript Inc), CleanCap (TriLink Biotechnologies LLC), a 5′-phosphorothiolate cap analog (Univ. Warszawski), or by use of alternative cap analogues.

As used herein, the term “3′-poly(A) tail” or “poly(A) tail” is typically a stretch of adenine nucleotides added to the 3′-end of the transcribed mRNA. It can, in some instances, be as short as 150 nucleotides or comprise up to about 500 adenine nucleotides. In some cases, the length of the 3′-poly(A) tail may be an essential element with respect to the stability of the individual mRNA. Said length can be of up to about 400 adenine nucleotides, e.g. from about 20 to about 400, preferably from about 50 to about 400, more preferably from about 50 to about 300, even more preferably from about 50 to about 250, most preferably from about 60 to about 250 adenine nucleotides. A poly(A) sequence is typically located at the 3′end of an mRNA. In the context of the present invention, a poly(A) sequence may be located within an mRNA or any other nucleic acid molecule, such as, e.g., in a vector, for example, in a vector serving as template for the generation of an RNA, preferably an mRNA, e.g., by transcription of the vector.

As used herein, a 5 ‘-cap is an entity, typically a modified nucleotide entity, which generally “caps” the 5’-end of a mature mRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. Further examples of 5′cap structures include glyceryl, inverted deoxy abasic residue (moiety), 4′, 5′ methylene nucleotide, I-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3 ‘-3’-inverted nucleotide moiety, 3′-3′-inverted abasic moiety, 3′-2′-inverted nucleotide moiety, 3 ‘-2’-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. Further modified 5′-CAP structures which may be used in the context of the present invention are CAP1 (methylation of the ribose of the adjacent nucleotide of m7GpppN), CAP2 (methylation of the ribose of the 2nd nucleotide downstream of the m7GpppN), CAP3 (methylation of the ribose of the 3rd nucleotide downstream of the m7GpppN), CAP4 (methylation of the ribose of the 4th nucleotide downstream of the m7GpppN), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine. A specifically preferred 5′-cap is the CleanCap structure, offered by TriLink.

As used herein, the term ‘S’-UTR′ typically refers to a particular section of messenger RNA (mRNA). It is located 5′ of the open reading frame of the mRNA. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be post-transcriptionally modified, for example by addition of a 5′-CAP. In the context of the present invention, a 5′-UTR corresponds to the sequence of a mature mRNA, which is located between the 5′-CAP and the start codon. Preferably, the 5′-UTR corresponds to the sequence, which extends from a nucleotide located 3′ to the 5′-CAP, preferably from the nucleotide located immediately 3′ to the 5′-CAP, to a nucleotide located 5′ to the start codon of the protein coding region, preferably to the nucleotide located immediately 5′ to the start codon of the protein coding region. The nucleotide located immediately 3′ to the 5′-CAP of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence, which corresponds to such RNA sequence. In the context of the present invention, the term “a 5′-UTR of a gene”, such as “a 5′-UTR of a TOP gene”, is the sequence, which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR. Preferably, the 5′-UTR used according to the present invention is heterologous to the coding region of the mRNA sequence. Even if 5′-UTR's derived from naturally occurring genes are preferred, also synthetically engineered UTR's may be used in the context of the present invention.

In the context of the present invention, a 3′-UTR is typically the part of an mRNA, which is located between the protein coding region (i.e. the open reading frame) and the 3′-terminus of the mRNA. A 3′-UTR of an mRNA is not translated into an amino acid sequence. The 3′-UTR sequence is generally encoded by the gene, which is transcribed into the respective mRNA during the gene expression process. In the context of the present invention, a 3′-UTR corresponds to the sequence of a mature mRNA, which is located 3′ to the stop codon of the protein coding region, preferably immediately 3′ to the stop codon of the protein coding region, and which extends to the 5′-side of the 3′-terminus of the mRNA or of the poly(A) sequence, preferably to the nucleotide immediately 5′ to the poly(A) sequence. The term “corresponds to” means that the 3′-UTR sequence may be an RNA sequence, such as in the mRNA sequence used for defining the 3′-UTR sequence, or a DNA sequence, which corresponds to such RNA sequence. In the context of the present invention, the term “a 3′-UTR of a gene”, such as “a 3′-UTR of an albumin gene”, is the sequence, which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR. Preferably, the 3′-UTR used according to the present invention is heterologous to the coding region of the mRNA sequence. Even if 3′-UTR's derived from naturally occurring genes are preferred, also synthetically engineered UTR's may be used in the context of the present invention.

An open reading frame (ORF) in the context of the invention may typically be a sequence of several nucleotide triplets, which may be translated into a peptide or protein. An open reading frame preferably contains a start codon, i.e. a combination of three subsequent nucleotides coding usually for the amino acid methionine (ATG), at its 5′-end and a subsequent region, which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present invention is preferably a nucleotide sequence, consisting of a number of nucleotides that may be divided by three, which starts with a start codon (e.g. ATG) and which preferably terminates with a stop codon (e.g., TAA, TGA, or TAG). The open reading frame may be isolated or it may be incorporated in a longer nucleic acid sequence, for example in a vector or an mRNA. An open reading frame may also be termed “protein coding region”.

An origin of replication (ORI) in the context of the invention may typically be a sequence of DNA at which replication is initiated on a chromosome, plasmid or virus. For small DNAs, including bacterial plasmids and small viruses, a single origin is sufficient. Larger DNAs may have many origins, and DNA replication is initiated at all of them. The origin of replication determines the vector copy number, which could typically be in the range of 25-50 copies/cell if the expression vector is derived from low-copy-number plasmids, or between 150 and 200 copies/cell if derived from a high-copy-number plasmid. The copy number influences the plasmid stability, i.e. the maintenance of the plasmid within the cells during cell division. A positive effect of a high copy number may be the greater stability of the plasmid when the random partitioning occurs at cell division. On the other hand, a high number of plasmids may decrease the growth rate, thus possibly allowing for cells with few plasmids to dominate the culture, since they grow faster. The origin of replication also may determine the plasmid's compatibility: its ability to replicate in conjunction with another plasmid within the same bacterial cell. Plasmids that utilize the same replication system cannot co-exist in the same bacterial cell. They are said to belong to the same compatibility group. The introduction of a new origin, in the form of a second plasmid from the same compatibility group, mimics the result of replication of the resident plasmid. Thus any further replication is prevented until after the two plasmids have been segregated to different cells to create the correct prereplication copy number.

Self-Replicating RNA Molecules

Recently, sa-mRNA (RNA replicon) vaccination is recognized as innovative nanotechnology-based vaccination strategy (Andries, Kitada, et al., 2015). As mentioned previously, unlike viral replicon particles (i.e. RNA encapsulated in viral capsid proteins), RNA replicon can be produced by in vitro transcription only. Thus, the whole manufacturing process is entirely cell-free, resulting in a therapeutic whose composition is precisely defined. RNA replicon vaccines have several attractive features, such as extending the duration (approximately 2 months) and magnitude of expression compared to their non-replicating counterparts (Kowalski et al., 2019). In addition, the intracellular replication of sa-mRNA is transient and the double-stranded RNA (dsRNA) induces interferon-mediated host-defense mechanisms by triggering pattern recognition receptors. This results in strong antigen-specific immune responses against the inserted target molecules. Thus, sa-mRNA vector systems are ideally suited for vaccine development because they provide high transient transgene expression and inherent adjuvant effects (Sahin et al., 2014).

The self-replicating RNA molecules of the invention are based on the genomic RNA of RNA viruses, but lack the genes encoding one or more structural proteins. The self-replicating RNA molecules are capable of being translated to produce non-structural proteins of the RNA virus and heterologous proteins encoded by the self-replicating RNA.

Self-replicating RNA molecules of the invention can be designed so that the self-replicating RNA molecule cannot induce production of infectious viral particles. This can be achieved, for example, by omitting one or more viral genes encoding structural proteins that are necessary to produce viral particles in the self-replicating RNA. For example, when the self-replicating RNA molecule is based on an alpha virus, such as Sindbis virus (SIN), Semliki forest virus and Venezuelan equine encephalitis virus (VEE), one or more genes encoding viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

One suitable system for achieving self-replication is to use an alphavirus-based RNA replicon. These +-stranded replicons are translated after delivery to a cell to give of a replicase (or replicase-transcriptase). The replicase is translated as a polyprotein which auto-cleaves to provide a replication complex which creates genomic −-strand copies of the +-strand delivered RNA. These −-strand transcripts can themselves be transcribed to give further copies of the +-stranded parent RNA and also to give a subgenomic transcript which encodes the desired gene product. Translation of the subgenomic transcript thus leads to in situ expression of the desired gene product by the infected cell. Suitable alphavirus replicons can use a replicase from a sindbis virus, a semliki forest virus, an eastern equine encephalitis virus, a venezuelan equine encephalitis virus, etc.

A preferred self-replicating RNA molecule thus encodes (i) a RNA-dependent RNA polymerase which can transcribe RNA from the self-replicating RNA molecule and (ii) a SARS-CoV-2 antigen as described herein. The polymerase can be an alphavirus replicase e.g. comprising alphavirus protein nsP4.

Whereas natural alphavirus genomes encode structural virion proteins in addition to the non-structural replicase polyprotein, it is preferred that an alphavirus based self-replicating RNA molecule of the invention does not encode alphavirus structural proteins. Thus the self-replicating RNA can lead to the production of genomic RNA copies of itself in a cell, but not to the production of RNA-containing alphavirus virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the invention and their place is taken by gene(s) encoding the desired gene product, such that the subgenomic transcript encodes the desired gene product rather than the structural alphavirus virion proteins. Therefore, in a particular embodiment, the self-replicating RNA molecule of the invention comprises a sequence encoding nonstructural alphavirus proteins and a sequence encoding a SARS-CoV-2 antigen. More in particular, the self-replicating RNA molecule of the invention comprises a sequence encoding the four nonstructural alphavirus proteins and a sequence encoding a SARS-CoV-2 antigen. Preferably, the self-replicating RNA molecule is derived from an alphavirus which has been engineered to lack the ability to produce at least one structural alphaviral protein. More preferably, the self-replicating RNA molecule is derived from an alphavirus which has been engineered to lack the ability to produce at least two, more preferably all, structural alphaviral proteins. In a particular embodiment, the self-replicating RNA molecule of the invention comprises, in 5′ to 3′ order (i) a 5′ sequence required for nonstructural protein-mediated amplification, (ii) a nucleotide sequence encoding alphavirus, particularly Venezuelan equine encephalitis virus, nonstructural proteins nsP1, nsP2, nsP3, and nsP4, (iii) a promotor which is operably linked to a heterologous nucleic acid sequence encoding a SARS-CoV-2 antigen, wherein the heterologous nucleic acid sequence replaces one or all of the alphavirus structural protein genes, (iv) a 3′ sequence required for nonstructural protein-mediated amplification, and (v) a polyadenylate tract.

Thus, a self-replicating RNA molecule useful with the invention may have two open reading frames. The first (5′) open reading frame encodes a replicase, particularly the nonstructural proteins of an alphavirus; and the second (3′) open reading frame encodes at least one SARS-CoV-2 antigen.

In one aspect, the self-replicating RNA molecule is derived from or based on an alphavirus. In other aspects, the self-replicating RNA molecule is derived from or based on a virus other than an alphavirus, preferably, a positive-stranded RNA viruses, and more preferably a picornavirus, flavivirus, rubivirus, pestivirus, hepacivirus, or calicivirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375). In a particular embodiment, the alphavirus is a Venezuelan Equine Encephalitis Virus (VEEV). In a more particular embodiment, the alphavirus is a live attenuated Venezuelan Equine Encephalitis Virus (VEEV), such as strain TC-83 or a strain having at least 90% sequence identity, preferably at least 95%, more preferably at least 97%, even more preferably at least 99%. Strain TC-83 is publicly available and its genome is present in Genbank under accession number L01443.1. Various genetically modified variants of alphaviruses have been generated that improve their use for self-replicating RNA molecule generation and vaccination, such as e.g. disclosed in US2015299728 A1, WO1999018226 A2 and U.S. Pat. No. 7,332,322 B2, all of which are incorporated herein by reference. In particular, it has been found to be beneficial to have a guanine as the third nucleotide in the 5′ UTR of the replicon and/or to have a Q739L mutation in Nonstructural Protein 2 (nsP2). Therefore, in a particular embodiment of the invention, the self-replicating RNA molecule comprises an A3G mutation in the 5′UTR. In another particular embodiment, the self-replicating RNA molecule comprises a Q739L mutation in Nonstructural Protein 2 (nsP2). In a preferred embodiment, the self-replicating RNA molecule comprises a sequence encoding the nonstructural proteins of an alphavirus, particularly VEEV, more particularly VEEV TC-83, wherein the self-replicating RNA molecule comprises an A3G mutation in the 5′UTR and a Q739L mutation in nsP2. In an even more preferred embodiment, the self-replicating RNA molecule encodes the nonstructural proteins nsP1, nsP2, nsP3 and nsP4 of VEEV TC-83, wherein preferably the Q739L mutation is present in nsP2. In one embodiment, the self-replicating RNA molecule encodes a protein comprising a sequence selected from the group consisting of SEQ ID NO: 11, SEQ ID NO: 12, SEQ ID NO: 13, or SEQ ID NO: 14, or in each instance a protein having at least 90% sequence identity, preferably at least 95%, more preferably at least 97%, even more preferably at least 99% thereto. In a further embodiment, the self-replicating RNA molecule encodes a protein comprising a sequence of SEQ ID NO: 11, SEQ ID NO: 12, SEQ ID NO: 13, and SEQ ID NO: 14, or in each instance a protein having at least 90% sequence identity, preferably at least 95%, more preferably at least 97%, even more preferably at least 99% thereto. In an even further embodiment, the self-replicating RNA molecule encodes a protein comprising a sequence of SEQ ID NO: 11, SEQ ID NO: 15, SEQ ID NO: 13, and SEQ ID NO: 14, or in each instance a protein having at least 90% sequence identity, preferably at least 95%, more preferably at least 97%, even more preferably at least 99% thereto. Between NsP3 and NsP4 there might be a stop codon with an infrequent read-through. This may result in the formation of various precursor forms of the nonstructural proteins being either nsP1-3 or nsP1-4. Therefore, the protein comprising SEQ ID NO: 18 (nsP3 as it is present in the nsP1-4 precursor) embraces SEQ ID NO: 13 (nsP3 as it is present in the nsP1-3 precursor, due to the stop codon with infrequent read-through) in some embodiments of the invention.

In a particular embodiment, the self-replicating RNA molecule of the invention comprises the RNA equivalent of SEQ ID NO: 10 or a sequence having at least 90% sequence identity, preferably at least 95%, more preferably at least 97%, even more preferably at least 99% thereto, optionally interrupted with one or more additional sequences. In a further embodiment, the self-replicating RNA molecule of the invention comprises the RNA equivalent of SEQ ID NO: 10 or a sequence having at least 90% sequence identity, preferably at least 95%, more preferably at least 97%, even more preferably at least 99% thereto, wherein said sequence is interrupted, preferably around nucleotide 7567 of said sequence, with one or more sequences encoding for a SARS-CoV-2 antigen, such as any one of SEQ ID NO: 1, SEQ ID NO: 2, SEQ ID NO: 8, and SEQ ID NO: 9.

As will be evident from the disclosures herein, preferably at least one SARS-CoV-2 antigen is a SARS-CoV-2 Spike protein antigen. Therefore, in a particular embodiment, the self-replicating RNA molecule of the invention comprises the RNA equivalent of SEQ ID NO: 10 or a sequence having at least 90% sequence identity, preferably at least 95%, more preferably at least 97%, even more preferably at least 99% thereto, wherein said sequence is interrupted with a sequence encoding for a SARS-CoV-2 Spike protein antigen. In a preferred embodiment, the self-replicating RNA molecule of the invention comprises the RNA equivalent of SEQ ID NO: 7, or a sequence having at least 90% sequence identity, preferably at least 95%, more preferably at least 97%, even more preferably at least 99% thereto, wherein said sequence is optionally interrupted with one or more additional sequences. Such additional sequences may encoding for a different SARS-CoV-2 antigen, such as a Nucleocapsid protein antigen and/or a Membrane protein antigen. In particular, such additional sequences may encode a protein comprising a sequence of SEQ ID NO: 8 or SEQ ID NO: 9. In another embodiment, such additional sequences may comprise the RNA equivalent of SEQ ID NO: 5 or SEQ ID NO: 6.

In one particular embodiment, the self-replicating RNA molecule of the invention comprises the RNA equivalent of SEQ ID NO: 7, or a sequence having at least 90% sequence identity, preferably at least 95%, more preferably at least 97%, even more preferably at least 99% thereto, wherein said sequence is interrupted with a sequence encoding for a SARS-CoV-2 Nucleocapsid protein antigen, such as a sequence encoding a protein comprising the sequence of SEQ ID NO: 8 or an antigenic fragment thereof. In another particular embodiment, the self-replicating RNA molecule of the invention comprises the RNA equivalent of SEQ ID NO: 7, or a sequence having at least 90% sequence identity, preferably at least 95%, more preferably at least 97%, even more preferably at least 99% thereto, wherein said sequence is interrupted with a sequence encoding for a SARS-CoV-2 Membrane protein antigen, such as a sequence encoding a protein comprising the sequence of SEQ ID NO: 9 or an antigenic fragment thereof.

The self-replicating RNA molecules described herein may be engineered to express multiple nucleotide sequences, from two or more open reading frames, thereby allowing co-expression of proteins, such as a two or more antigens together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine, or in gene therapy applications.

SARS-CoV-2 Antigen

For use in the invention any SARS-CoV-2 protein antigen can be used, meaning that the self-replicating RNA molecule comprises a sequence encoding the SARS-CoV-2 protein antigen. Of particular interest are SARS-CoV-2 protein antigens of Spike protein (also referred to as S protein), Membrane protein (also referred to as M protein) or Nucleocapsid protein (also referred to as N protein). The term “antigen” encompasses antigenic fragments. For example, a SARS-CoV-2 Spike protein antigen encompasses the full-length Spike protein as well as antigenic fragments thereof, such as the Receptor-Binding Domain (RBD) of the Spike protein and fusion molecules comprising such antigenic fragments.

As will be understood from the disclosures herein, the self-replicating RNA molecule preferably comprises a SARS-CoV-2 Spike protein antigen. In a further embodiment, the SARS-CoV-2 Spike protein antigen is a truncated form of the Spike protein. In one particular embodiment, the SARS-CoV-2 Spike protein antigen comprises at least 15, in particular at least 20, more in particular at least 25 consecutive amino acids of SEQ ID NO: 16. In a further particular embodiment, the SARS-CoV-2 Spike protein antigen comprises SEQ ID NO: 16 or an amino acid sequence having at least 95% sequence identity to SEQ ID NO: 16, more preferably at least 97%, even more preferably at least 99%. In a further embodiment, sequence encoding the SARS-CoV-2 Spike protein antigen comprises the RNA equivalent of SEQ ID NO: 17 or a nucleotide sequence having at least 95% sequence identity to the RNA equivalent of SEQ ID NO: 17, more preferably at least 97%, even more preferably at least 99%.

In another embodiment, the SARS-CoV-2 Spike protein antigen comprises the Receptor-Binding Domain (RBD). In yet another particular embodiment, the SARS-CoV-2 Spike protein antigen is a truncated form of the Spike protein comprising the RBD. In a further embodiment, the SARS-CoV-2 Spike protein antigen consists essentially of the RBD, optionally fused to another peptide. Therefore, in a particular embodiment, the SARS-CoV-2 Spike protein antigen comprises SEQ ID NO: 1 or an amino acid sequence having at least 95% sequence identity to SEQ ID NO: 1, more preferably at least 97%, even more preferably at least 99%. In a further embodiment, the SARS-CoV-2 Spike protein antigen consists essentially of SEQ ID NO: 1, optionally fused to another peptide, or an amino acid sequence having at least 95% sequence identity to SEQ ID NO: 1, more preferably at least 97%, even more preferably at least 99%. In another embodiment, the sequence encoding a SARS-CoV-2 Spike protein antigen encodes the sequence of SEQ ID NO: 1 or an amino acid sequence having at least 95% sequence identity to SEQ ID NO: 1, more preferably at least 97%, even more preferably at least 99%. In another embodiment, the sequence encoding the SARS-CoV-2 Spike protein antigen encodes an amino acid sequence consisting essentially of SEQ ID NO: 1, optionally fused to another peptide, or an amino acid sequence having at least 95% sequence identity to SEQ ID NO: 1, more preferably at least 97%, even more preferably at least 99%. In a further embodiment, sequence encoding the SARS-CoV-2 Spike protein antigen comprises the RNA equivalent of SEQ ID NO: 3 or a nucleotide sequence having at least 95% sequence identity to the RNA equivalent of SEQ ID NO: 3, more preferably at least 97%, even more preferably at least 99%.According to the invention, a sequence encoding the SARS-CoV-2 Spike protein antigen may be preceded by leader peptides, such as, but not limited to, human tissue plasminogen activator leader peptide (an amino acid sequence having at least 95% sequence identity to SEQ ID NO: 19) and IgG kappa light chain leader sequence.

In a further embodiment, the Spike protein is fused to an immune stimulating protein, optionally through a linker sequence. Different immune stimulating protein can be used for the application of the invention. A preferred immune stimulating protein for use in the invention is C3d-p28, particularly C3d-p28.6, such as described in Zhang et al. (Vaccine 2011, 29:629-635). Therefore, in a particular embodiment, the SARS-CoV-2 Spike protein antigen comprises the Receptor-Binding Domain (RBD) fused to C3d-p28. In yet another particular embodiment, the SARS-CoV-2 Spike protein antigen is a truncated form of the Spike protein comprising the RBD fused to C3d-p28. In a further embodiment, the SARS-CoV-2 Spike protein antigen consists essentially of the RBD fused to C3d-p28, optionally fused to another peptide. Therefore, in a particular embodiment, the SARS-CoV-2 Spike protein antigen comprises SEQ ID NO: 2 or an amino acid sequence having at least 95% sequence identity to SEQ ID NO: 2, more preferably at least 97%, even more preferably at least 99%. In a further embodiment, the SARS-CoV-2 Spike protein antigen consists essentially of SEQ ID NO: 2, optionally fused to another peptide, or an amino acid sequence having at least 95% sequence identity to SEQ ID NO: 2, more preferably at least 97%, even more preferably at least 99%. In another embodiment, the sequence encoding a SARS-CoV-2 Spike protein antigen encodes the sequence of SEQ ID NO: 2 or an amino acid sequence having at least 95% sequence identity to SEQ ID NO: 2, more preferably at least 97%, even more preferably at least 99%. In another embodiment, the sequence encoding the SARS-CoV-2 Spike protein antigen encodes an amino acid sequence consisting essentially of SEQ ID NO: 2, optionally fused to another peptide, or an amino acid sequence having at least 95% sequence identity to SEQ ID NO: 2, more preferably at least 97%, even more preferably at least 99%. In a further embodiment, the sequence encoding the SARS-CoV-2 Spike protein antigen comprises the RNA equivalent of SEQ ID NO: 4 or a nucleotide sequence having at least 95% sequence identity to the RNA equivalent of SEQ ID NO: 4, more preferably at least 97%, even more preferably at least 99%.

Preferably the SARS-CoV-2 Spike protein antigen is combined with another SARS-CoV-2 antigen. It has been found that this allows for a greater efficacy of vaccination. SARS-CoV-2 antigens may be combined by, for example, providing them in different self-replicating RNA molecules as described herein or by providing a single self-replicating RNA molecule as described herein which encodes for the different antigens.

Therefore, in one aspect of the present invention a combination is provided comprising a sequence encoding a SARS-CoV-2 Spike protein antigen and a sequence encoding a further SARS-CoV-2 protein antigen. Preferably, the further SARS-CoV-2 antigen is a SARS-CoV-2 Nucleocapsid protein antigen.

According to one embodiment of the combination according to present invention, the sequence encoding the SARS-CoV-2 Spike protein antigen and the sequence encoding a further SARS-CoV-2 protein antigen are comprised in the same self-replicating RNA molecule. In one particular embodiment, the self-replicating RNA molecule of the invention encodes for two SARS-CoV-2 antigens, preferably (a) a SARS-CoV-2 Spike protein antigen and (b) a SARS-CoV-2 Nucleocapsid protein antigen and/or a SARS-CoV-2 Membrane protein antigen. In one particular embodiment, the self-replicating RNA molecule of the invention encodes for (a) a SARS-CoV-2 Spike protein antigen and (b) a SARS-CoV-2 Nucleocapsid protein antigen. In another particular embodiment, the self-replicating RNA molecule of the invention encodes for (a) a SARS-CoV-2 Spike protein antigen and (b) a SARS-CoV-2 Membrane protein antigen.

According to another embodiment of the combination according to the present invention, the sequence encoding the SARS-CoV-2 Spike protein antigen and the sequence encoding a further SARS-CoV-2 protein antigen are comprised in separate self-replicating RNA molecules. In one particular embodiment, the combination comprises two different self-replicating RNA molecules, each encoding for one SARS-CoV-2 antigen, preferably (a) a SARS-CoV-2 Spike protein antigen and (b) a SARS-CoV-2 Nucleocapsid protein antigen and/or a SARS-CoV-2 Membrane protein antigen. In one particular embodiment, the combination comprises one self-replicating RNA molecule encoding the SARS-CoV-2 Spike protein antigen, and a second self-replicating RNA molecule encoding the SARS-CoV-2 Nucleocapsid protein antigen. In another particular embodiment, the combination comprises one self-replicating RNA molecule encoding the SARS-CoV-2 Spike protein antigen, and a second self-replicating RNA molecule encoding the SARS-CoV-2 Membrane protein antigen.

Delivery of Self-Replicating RNA Molecules

According to the common classification, two types of vectors can be used to deliver genetic material to the target cells. On one hand, viral vectors, that mimic the behavior of their precursor viruses, are used. Different types of vectors, including retroviral, lentiviral, adenoviral, and adeno-associated viruses have been employed and are even being approved for clinical use in Europe. On the other hand, there are non-viral vectors that can be described according to their composition. The most cited are lipoplexes (lipid+DNA or RNA) and polyplexes (polymer+DNA or RNA) (Perez Ruiz de Garibay, 2016). Genetic material can also be inserted via electroporation. This process of introducing foreign substances from the cell works by applying electrical fields to cells in order to increase the permeability of the cell membrane. This project will focus on researching non-viral delivery systems.

The self-replicating RNA of this invention is suitable for delivery in a variety of modalities, including naked RNA delivery or in combination with lipids, polymers or other compounds that facilitate entry into the cells. Self-replicating RNA molecules of the present invention can be introduced into target cells or subjects using any suitable technique, e.g., by direct injection, microinjection, electroporation, lipofection, biolystics, etc.

The self-replicating RNA can be delivered as naked RNA (e.g. merely as an aqueous solution of RNA) but, to enhance entry into cells and subsequent intercellular effects, the self-replicating RNA is preferably administered in combination with a delivery system such as a liposome or other nanoparticles.

In order to create a liposome, various amphiphilic lipids can form bilayers in an aqueous environment to encapsulate an RNA-containing aqueous core. These lipids can have an anionic, cationic or zwitterionic hydrophilic head group. Some phospholipids are anionic whereas other are zwitterionic.

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMPG (anionic, saturated). Where a mixture of lipids is used, not all the component lipids in the mixture need to be amphiphilic e.g. one or more amphiphilic lipids can be mixed with cholesterol. RNA is preferably encapsulated within the liposomes, and so the liposome forms an outer layer around an aqueous RNA-containing core.

An efficient delivery system that formulates the mRNA into nanoparticles can increase the cellular uptake and protect the mRNA against RNases. The latter is especially important if the mRNA is administered by routes that bring the mRNA in contact with biofluids like blood or mucus. Lipid-based carriers, polymer-based carriers, and hybrid and other nanoformulations, such as carbon nanotubes (CNTs) or dendriplexes (dendrimers+DNA/RNA) have been evaluated in vivo (Perez Ruiz de Garibay, 2016). Most of the used lipid formulations of mRNA are rather simple and involve an ordinary mixing of the lipids with the mRNA. Additionally, the resulting mRNA-liposome complexes are often positively charged at physiologic pH and hence prone to interaction with negatively charged (macro)molecules present in biological fluids. Furthermore, such charged mRNA-complexes are rapidly captured by immune cells (Zhong et al., 2018).

Lipid nanoparticles (LNPs) were introduced as a more advanced lipid formulation for mRNA (Moss et al., 2007; Islam et al., 2015; Kauffman et al., 2016; Granot & Peer, 2017; Hajj & Whitehead, 2017; B. Li et al., 2019), and they are considered as ideal candidates for the in vivo delivery of mRNA therapeutics. Characteristic for LNPs is that they encapsulate the mRNA into nanoparticles that are quasi neutral at physiologic pH but become positive at acidic pH. This prevents that charged (macro)molecules in biological fluids like e.g. albumin massively bind to the LNPs. On the other hand, the protonation of the pH-responsive lipids in the acidic endosomes promotes the endosomal escape of the mRNA. The pH responsiveness of LNPs is due to the presence of ionizable lipids with an appropriate pKa, i.e. the lipids are positively charged at low pH, but neutral around physiologic pH. Together with a dedicated mixing procedure, these pH responsive lipids allow an efficient encapsulation of the mRNA.

LNP-cmRNA-based systems could represent a powerful platform technology for correction of cystic fibrosis and other monogenic disorders. Administration of LNP-cmRNA to the lung can be performed by aerosol inhalation/nebulization, as a non-invasive tool. Johler et al., (2015) have shown that aerosolization of cationic IVT mRNA complexes, which does neither affect the protein duration nor the toxicity of the cationic complexes, constitute a potentially powerful means to transfect cells in the lung with the purpose of protein replacement for genetic diseases such as cystic fibrosis, while bringing along the advantages of IVT mRNA as compared to pDNA as transfection agent (Johler et al., 2015). Robinson et al., (2018) established that intranasal administration of mRNA-LNPs encoding the wildtype cystic fibrosis transmembrane conductance regulator (CFTR) restored the CFTR-mediated chloride secretion in conductive airways of CFTR knockout mice (Robinson et al., 2018b). Also, for certain applications, specific cell types or tissues need be targeted. For instance, recently, a rare cell type, the Foxi1+ pulmonary ionocyte was identified as the major source of transcripts of the cystic fibrosis transmembrane conductance regulator in both mouse (Cftr) and human (CFTR) (Montoro et al., 2018). This new cell type could be the ideal target for mRNA therapeutics as targeted delivery of mRNA therapeutics is expected to become a hot topic in the mRNA delivery field, providing major opportunities for innovation.

Additionally, lipid-based micro-/nanoparticles can possess several of the desired characteristics of an interesting Antigen (Ag)-delivery system for vaccination as they are biocompatible, can overcome physiological barriers at mucosae, promote Ag crossing of the epithelium and uptake by APCs, protect the associated payload, are adequate for incorporating adjuvants and may display mucoadhesive properties (Corthésy & Bioley, 2018). Geall et al. (2012) showed that lipid nanoparticle encapsulated sa-mRNA vaccines elicited functional immune responses against antigens from HIV and respiratory syncytial virus (Geall et al., 2012).

The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491, all of which are incorporated by reference herein in their entirety.

In a particularly preferred embodiment, the self-replicating RNA molecule of the invention is encapsulated in a lipid nanoparticle.

All embodiments described above for the delivery of self-replicating RNA molecules equally apply to the delivery of a combination of several different self-replicating RNA molecules, as described above. In particular, when sequences encoding multiple SARS-CoV-2 protein antigens comprised in separate self-replicating RNA molecules are to be delivered, these can be delivered in one single liposome, or in separate liposomes. Optionally, the separate liposomes can be mixed prior to delivery in order to facilitate further handling of the formulation. Said separate liposomes can have the same composition or different compositions so as to efficiently encapsulate, bind or adsorb the different self-replicating RNA molecules. Therefore, one particular embodiment of the present inventions relates to a combination as described above, wherein the sequence encoding the SARS-CoV-2 Spike protein antigen and the sequence encoding a further SARS-CoV-2 protein antigen are comprised in different self-replicating RNA molecules and wherein both self-replicating RNA molecules are encapsulated in, bound to or adsorbed on one single liposome. When two or more different self-replicating RNA molecules are used in the present invention, the liposome encapsulating the different self-replicating RNA molecules may have the same or different composition. Therefore, in a particular embodiment, the present invention provides a first self-replicating RNA molecule comprising a sequence encoding a first SARS-CoV-2 protein antigen and a second self-replicating RNA molecule comprising a sequence encoding a second SARS-CoV-2 protein antigen, wherein the first self-replicating RNA molecule is encapsulated in, bound to or absorbed on a first liposome and the second self-replicating RNA molecule is encapsulated in, bound to or absorbed on a second liposome. The first and second liposome may have the same or a different composition, in particular they may comprise the same or different lipids. In a preferred embodiment, the first self-replicating RNA molecule is encapsulated in a first LNP and the second self-replicating RNA molecule is encapsulated in a second LNP. In one particular embodiment, the composition of the first LNP is different from the composition of the second LNP. This may be obtained, e.g. by encapsulating the first self-replicating RNA molecule in a first LNP encapsulation mixture and encapsulating the second self-replicating RNA molecule in a second LNP encapsulation mixture. Thereafter, the first and second encapsulated self-replicating RNA molecules may be provided in different compositions or they can be combined to obtain a composition comprising both encapsulated self-replicating RNA molecules.

In another particular embodiment of the present inventions relates to a as described above, wherein the sequence encoding the SARS-CoV-2 Spike protein antigen and the sequence encoding a further SARS-CoV-2 protein antigen are comprised in different self-replicating RNA molecules and wherein the sa-RNA molecule comprising the sequence encoding the SARS-CoV-2 Spike protein antigen and the sa-RNA molecule comprising the sequence encoding a further SARS-CoV-2 protein antigen are encapsulated in, bound to or adsorbed on separate liposomes. The separate liposomes can be delivered in two separate compositions. Optionally, the separate liposomes can also be combined in one single composition prior to delivery.

Pharmaceutical Compositions

The invention relates to pharmaceutical compositions comprising S, N or M protein-encoding self-replicating RNA sequences or a combination of two or three protein-encoding self-replicating RNA sequences. Therefore, in a particular embodiment, the present invention provides a pharmaceutical composition comprising a self-replicating RNA molecule as described herein and one or more pharmaceutically acceptable carriers. A pharmaceutically acceptable carrier and a suitable delivery system or vehicle, such as liposomes, are typically included. However, the mRNA can also be delivered naked, with or without electroporation. At least one adjuvant can be included in the pharmaceutical composition and if desired other adjuvants or other pharmaceutical components, such as excipients can be included. These components can be used as anti-viral vaccines. If SARS-CoV-2 antigens are provided in different self-replicating RNA molecules, these molecules may be present in the same or in a different composition.

Pharmaceutically acceptable carriers are determined in part by the particular composition being administered, as well as by the particular method used to administer the composition. Subsequently, there is a wide variety of suitable formulations of pharmaceutical compositions of the present invention. The self-replicating RNA molecule can be encapsulated in, bound to or absorbed on a cationic lipid, a lipid nanoparticle, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, a cationic nano-emulsion or combinations thereof.

The pharmaceutical compositions are preferably sterile and may be sterilized by conventional sterilization techniques.

The compositions may contain pharmaceutically acceptable auxiliary substances, to approximate physiological conditions, such as pH adjusting and/or buffering agents and tonicity adjusting agents, for example sodium acetate, sodium chloride, potassium chloride, calcium chloride, sodium lactate and the like.

The pH of the pharmaceutical compositions is preferably between 5.0 and 9.5, e.g. between 6.0 and 8.0.

The tonicity of the pharmaceutical composition of the invention may have to be adjusted with sodium salts, for example, sodium chloride. The tonicity of a pharmaceutical composition for parenteral administration is typically 0,9% or 9 mg/ml NaCl.

Pharmaceutical compositions of the invention may have an osmolality of between 200 mOsm/kg and 400 mOsm/kg, e.g. between 240-360 mOsm/kg or between 290-310 mOsm/kg.

Preservative-free vaccines are preferred. However, if desired, the pharmaceutical compositions of the invention may include one or more preservatives, such as phenol and 2-phenoxyethanol. Thiomersal, a mercury containing preservative, should be avoided as mercury-free compositions are preferred.

Pharmaceutical compositions of the invention are preferably non-pyrogenic e.g. containing <1 EU (endotoxin unit, a standard measure) per dose and preferably <0.1 EU per dose. Pharmaceutical compositions of the invention are preferably gluten free.

The concentration of self-replicating RNA in the pharmaceutical composition can vary and will be selected based on fluid volumes, viscosities, body weight and other considerations in accordance with the particular mode of administration. The concentration of self-replicating RNA in the pharmaceutical composition will have proved to be effective for prevention, either in a single dose or as part of a series of doses. The amount varies depending upon the health, physical condition, age and taxonomic group of the individual to be treated (e.g. non-human primate, primate, etc.), the capacity of the individual's immune system to react to the antigen encoded protein or peptide, the condition to be treated and other relevant factors. The self-replicating RNA content of compositions of the invention will generally be expressed in terms of the amount of RNA per dose. A preferred dose has between 0.1 to 100 μg self-replicating RNA, preferably between 0.5 to 90 μg self-replicating RNA, preferably between 0.1 to 75 μg self-replicating RNA, preferably between 0.1 to 50 μg self-replicating RNA, preferably between 0.5 to 50 μg self-replicating RNA, preferably between 0.5 to 25 μg self-replicating RNA, more preferably between 0.5 to 10 μg self-replicating RNA, more preferably between 1 and 10 μg, even more preferably between 1 and 5 μg self-replicating RNA and expression can be seen at much lower levels (e.g. 0.05 μg self-replicating RNA/dose during in vitro use).

Suitable routes of administration include enteral, parenteral and topical administration.

Parenteral administrations include intra-articular, intravenous, intraperitoneal, intramuscular, intradermal or subcutaneous injection. Intramuscular, intradermal or subcutaneous administrations are preferred. Formulations suitable for parenteral administrations include aqueous and non-aqueous, isotonic, sterile injection solutions, which can contain antioxidants, buffers, preservatives and solutes that render the formulation isotonic with the blood of the intended recipient.

Formulations suitable for parenteral administration, such as—but not limited to—intraarticular, intravenous, intraperitoneal, intramuscular, intradermal or subcutaneous injection, include aqueous and non-aqueous, isotonic sterile injection solutions or suspensions, which can contain antioxidants, buffers, bacteriostats, and solutes that render the formulation isotonic with the blood of the intended recipient, The formulation of self-replicating RNA molecules can be presented in unit-dose or multi-dose sealed containers, such as ampoules and vials. Injection solutions and suspensions can be prepared from sterile powders, granules, and tablets. Cells transfected by the self-replicating RNA molecules can also be administered intravenously or parenterally.

Said formulation or vaccine can be administered as a single dose or as a multi-dose, requiring a series of two or more doses, administered within a pre-defined timespan. Such timespan may be a week, two weeks, three weeks, four weeks, five weeks, six weeks, seven weeks, 8 weeks, 9 weeks, 10 weeks, 11 weeks up until one year. In an embodiment, the formulation or vaccine is administered periodically, such as annually or bi-annually.

A suited dose may be between 0.05 and 1 ml, more preferably between 0.25 and 0.75 ml, such as 0.5 ml.

If the pharmaceutical formulation consists of an emulsion, the self-replicating RNA molecules and emulsion can be mixed by simple shaking. Other techniques, such as passing a mixture of the emulsion and solution or suspension to the self-replicating RNA molecules rapidly through a small opening (such as a hypodermic needle), can be used to mix the pharmaceutical formulation.

Formulations, suitable for oral administration, can consist of (a) liquid solutions, (b) capsules, sachets or tablets, each containing a predetermined amount of the active ingredient, as liquids, solids, granules or gelatin, (c) suspensions in an appropriate liquid, and (d) suitable emulsions. Liquid solutions consist of an effective amount of the packaged nucleic acid suspended in diluents, such as water, saline or PEG 400. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. To protect orally administered self-replicating RNA molecules, the molecules are either complexed with a composition to render it resistant to acidic and enzymatic hydrolysis or by packaging the self-replicating RNA molecules in an appropriately resistant carrier, such as a liposome. Additionally, the pharmaceutical compositions can be encapsulated in e.g. liposomes or in a formulation that provides for slow release of the active ingredient.

Topical compositions, including aerosol formulations to be administered via inhalation, can be prepared as well. Suitable compositions consist of the self-replicating RNA molecules, alone or in combination with other suitable components. Aerosol formulations can be placed into pressurized acceptable propellants, such as dichlorodifluoromethane, propane nitrogen and the like.

Suitable suppository formulations contain of the self-replicating RNA molecule and a suppository base. Suitable suppository bases include natural or synthetic triglycerides or paraffin hydrocarbons. It is also possible to use gelatin rectal capsules filled with a combination of the self-replicating RNA with a suitable base, for example, liquid triglycerides, polyethylene glycols, and paraffin hydrocarbons.

All embodiments described above for pharmaceutical compositions comprising self-replicating RNA molecules equally apply to pharmaceutical compositions comprising a combination of several different self-replicating RNA molecules, as described above. In particular, when sequences encoding multiple SARS-CoV-2 protein antigens comprised in separate self-replicating RNA molecules, these can be present in one single formulation, or in separate formulations. Preferably, when sequences encoding multiple SARS-CoV-2 protein antigens comprised in separate self-replicating RNA molecules are present in separate formulations, these separate formulations can be mixed prior to delivery in order to facilitate further handling. Said separate formulations may comprise the same pharmaceutically acceptable carriers. Alternatively, the separate formulations can comprise different pharmaceutically acceptable carriers.

Separate formulations as described above can be administered using the same administration route or using different administration routes. Preferably, both formulations are administered using the same administration route.

Methods of Treatment and Medical Uses

Self-replicating RNA molecules of the present invention can be delivered to a vertebrate, such as a mammal (including a human) for a variety of therapeutic or prophylactic purposes, such as to induce a therapeutic or prophylactic immune response. The present invention is also directed to methods of stimulating an immune response in or treating a subject comprising administering to the subject one or more self-replicating RNA molecules as described herein in an amount effective to achieve the desired treatment effect, such as an amount sufficient to produce an amount of the encoded exogenous gene product sufficient to induce an immune response, to regulate expression of endogenous genes, or to provide therapeutic benefit. The subject is preferably an animal, a mammal, a fish, a bird and more preferably a human. Suitable animal subjects include, for example, cattle, pigs, horses, deer, sheep, goats, bison, rabbits, cats, dogs, chickens, ducks, turkeys, and the like.

The present invention is also directed to methods of inducing an immune response in a host animal comprising administering to the animal one or more self-replicating RNA molecules described herein in an amount effective to induce an immune response. Preferably said immune response is elicited in a subject against a coronavirus infection, preferably SARS-CoV-2. Preferably, the self-replicating RNA molecule encode a pathogen antigen. The host animal is preferably a mammal, more preferably a human. Preferred routes of administration are described above. The methods can be used to raise a booster response. The self-replicating RNA molecules and compositions described herein may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. As described above, one of the attractive features of RNA replicon vaccines is the extended duration of the immune response in the host animal to with the vaccine has been administered. Therefore, the time of administration between the initial administration of the prophylactic composition and the booster may be, but is not limited to, 1 week, 2 weeks, 3 weeks, 1 month, 2 months, 3 months, 6 months or 1 year. Preferably, the time of administration between the initial administration of the prophylactic composition and the booster is 2 or 3 months. The present invention relates to methods of immunizing a subject against SARS-CoV-2 comprising administering to the subject one or more self-replicating RNA molecules that encode a SARS-CoV-2 antigen in an amount effective to induce a protective immune response. The host animal is preferably a mammal, more preferably a human.

Preferably, the self-replicating RNA molecules of the invention that encode a SARS-CoV-2 antigen induce protective immunity or an immune response when administered to a subject.

Preferred routes of administration include, but are not limited to, intramuscular, intraperitoneal, intradermal, subcutaneous, intravenous, intraarterial, and intraocular injection. Oral and transdermal administration, as well as administration by inhalation or suppository is also contemplated. Particularly preferred routes of administration include intramuscular, intradermal and subcutaneous injection. Particularly preferred is intramuscular injection. According to some embodiments of the present invention, the self-replicating RNA molecules are administered to a host animal using a needleless injection device, which are well-known and widely available. Self-replicating RNA molecules of the invention can also be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have been transfected with the self-replicating RNA molecule. The appropriate number of cells to deliver to a patient will vary with patient conditions, and desired effect, which can be determined by a skilled artisan. Self-replicating RNA molecules, such as those that encode a pathogen antigen and thus are suitable for use to induce an immune response, can be introduced directly into a tissue, such as muscle. Other methods such as “biolistic” or particle-mediated transformation are also suitable for introduction of the self-replicating RNA into cells of a mammal according to the invention. These methods are useful not only for in vivo introduction of RNA into a mammal, but also for ex vivo modification of cells for reintroduction into a mammal.

It is contemplated that the self-replicating RNA molecule of this invention can be used in conjunction with whole cell or viral immunogenic compositions as well as with purified antigens, immunogens or protein subunit or peptide immunogenic compositions. It is sometimes advantageous to employ a self-replicating RNA vaccine that is targeted for a target cell type.

An effective amount of self-replicating RNA is administered to the subject in accordance with the methods described herein, either in a single dose or as part of a series of doses. As described herein, this amount varies depending upon the health and physical condition of the individual to be treated, the condition to be treated, and other relevant factors. It is expected that the amount will fall in a relatively broad range that can be determined by a skilled clinician based on the factors discussed herein, and other relevant factors.

Self-replicating RNA molecules vaccines of the invention that express the polypeptides, can be packaged in packs, dispenser devices, and kits. For example, packs or dispenser devices that contain one-or-more-unit dosage forms are provided. Typically, instructions for administration will be provided with the packaging, along with a suitable indication on the label that the self-replicating RNA molecule is suitable for treatment of an indicated condition. For example, the label may state that the self-replicating RNA molecule within the packaging is useful for treating a particular infectious disease, autoimmune disorder, tumor, or for preventing or treating other diseases or conditions that are mediated by, or potentially susceptible to, a mammalian immune response.

Vectors and RNA Constructs

A vector of the present invention may comprise an antigen, wherein said antigen sequence encodes for a SARS-CoV-2 Spike protein or a truncated form thereof, or wherein said antigen sequence encodes for a SARS-CoV-2 Nucleocapsid (N) antigen and wherein said antigen is downstream of a promoter sequence, preferably an alphavirus derived subgenomic promoter (SGP).

The vector may comprise basic elements including a cap, 5′ UTR, 3′ UTR, and poly(A) tail of variable length downstream of an antigen sequence. The vector may further comprise an origin of replication, and promoter sequences such as a T7 or SP6 promoter, as well as a selection gene, e.g. encoding an antibiotic, for the purpose of producing the vector in an expression system.

In an embodiment, a vector may comprise Venezuelan equine encephalitis virus (VEEV) carrying TC-83 strain genome used as replicon backbone to drive self-amplifying RNA expression. Self-amplifying RNA (saRNA) may encode four non-structural proteins (nsP1-4) and a subgenomic promoter sequence. nsP1-4 encode a replicase responsible for amplification of the saRNA that enable lower doses than non-replicating mRNA.

The aforementioned vector may further comprise a 5′ cap (e.g., 7mG(5′)ppp(5′)NImpNp). The non-structural proteins according to the current invention preferably comprise an A3G mutation in the 5′UTR and/or a Q739L mutation in Nonstructural Protein 2 (nsP2).

A sequence encoding the Spike protein antigen may be cloned after the SGP promoter, whereby the Spike protein antigen may be a truncated form of the Spike protein comprising the Receptor-Binding Domain (RBD). Alternatively, a sequence encoding SARS-CoV-2 Nucleocapsid protein antigen may be cloned after the SGP promoter. Preferentially, following delivery to the cytoplasm, translation of the saRNA may produce the non-structural proteins 1-4 (nsP 1-4) that form the RNA-dependent RNA polymerase (RDRP). RDRP is responsible for replication of the saRNA producing copies of the saRNA. Multiple copies of the subgenomic RNA are hence produced from each saRNA originally delivered. This leads to translation of many more copies of the antigen when compared to a non-amplifying RNA. The vector according to the current invention may be plasmid DNA or linearized DNA. For that purpose, a plasmid may be comprised of a restriction enzyme (RE) site allowing the linearization of said plasmid.

In an embodiment, the antigen sequence encodes for a SARS-CoV-2 Spike protein or a truncated form thereof, wherein said Spike protein or truncated form of the Spike protein comprises the Receptor-Binding Domain (RBD), or encodes for a SARS-CoV-2 Nucleocapsid (N) antigen.

A preferred self-replicating RNA molecule of the invention encodes a RNA-dependent RNA polymerase which can transcribe RNA from the self-replicating RNA molecule and a SARS-CoV-2 antigen as described herein. The polymerase can be an alphavirus replicase e.g. comprising alphavirus protein nsP4.

In an embodiment, the self-replicating RNA molecule of the invention comprises a sequence encoding nonstructural alphavirus proteins and a sequence encoding a SARS-CoV-2 antigen. More in particular, the self-replicating RNA molecule of the invention comprises a sequence encoding the four nonstructural alphavirus proteins and a sequence encoding a SARS-CoV-2 antigen. Said SARS-CoV-2 antigen is preferably selected from SARS-CoV-2 Spike protein, a truncated form of the SARS-CoV-2 Spike protein comprising the Receptor-Binding Domain (RBD), SARS-CoV-2

Spike protein Receptor-Binding Domain fused to C3d-p28.6, SARS-CoV-2 membrane protein (M) or a SARS-CoV-2 Nucleocapsid protein (N).

Preferably, the self-replicating RNA molecule is derived from an alphavirus which has been engineered to lack the ability to produce at least one structural alphaviral protein. More preferably, the self-replicating RNA molecule is derived from an alphavirus which has been engineered to lack the ability to produce at least two, more preferably all, structural alphaviral proteins. In a particular embodiment, the self-replicating RNA molecule of the invention comprises a nucleotide sequence encoding alphavirus, particularly Venezuelan equine encephalitis virus, nonstructural proteins nsP1, nsP2, nsP3, and nsP4, preferentially comprising an A3G mutation in the 5′UTR and/or a Q739L mutation in Nonstructural Protein 2 (nsP2)

Preferred sequences for use in the invention and actually used in the examples:

SEQ ID NO: 1. Amino acid sequence of SARS-COV-2 Receptor-Binding Domain (S-
RBD), preceeded by the human tissue plasminogen activator leader peptide:
MDAMKRGLCCVLLLCGAVFVSPRVQPTESIVRFPNITNLCPFGEVFNATRFASVYAWNRKRIS
NCVADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCNGV
EGFNCYFPLQSYGFQPTNGVGYQPYRVVVLSFELLHAPATVCGPKKSTNLVKNKCVNF
SEQ ID NO: 2. Amino acid sequence of SARS-COV-2 Receptor-Binding Domain fused
to C3d-p28.6 (S-RBD-C3d-p28.6), preceeded by the human tissue plasminogen
activator leader peptide:
MDAMKRGLCCVLLLCGAVFVSPRVQPTESIVRFPNITNLCPFGEVFNATRFASVYAWNRKRIS
NCVADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCNGV
EGFNCYFPLQSYGFQPTNGVGYQPYRVVVLSFELLHAPATVCGPKKSTNLVKNKCVNFGGGG
SGGGGSKFLTTAKDKNRWEDPGKQLYNVEATSYAGGGGSGGGGSKFLTTAKDKNRWEDP
GKQLYNVEATSYAGGGGSGGGGSKFLTTAKDKNRWEDPGKQLYNVEATSYAGGGGSGGG
GSKFLTTAKDKNRWEDPGKQLYNVEATSYAGGGGSGGGGSKFLTTAKDKNRWEDPGKQLY
NVEATSYAGGGGSGGGGSKFLTTAKDKNRWEDPGKQLYNVEATSYA
SEQ ID NO: 3. DNA equivalent of protein coding sequence of SARS-COV-2 S-RBD in
RNA sequence (All sequences are given in ″DNA format″. In the RNA vectors all T's
are changed to U's):
ATGGATGCTATGAAGAGGGGCCTGTGCTGCGTGCTGCTTCTGTGTGGCGCTGTGTTCGT
GTCCCCTAGAGTGCAGCCTACCGAGAGCATCGTGCGGTTCCCCAACATCACCAATCTGTG
CCCTTTCGGCGAGGTGTTCAACGCCACCAGATTCGCCTCTGTGTACGCCTGGAACCGGAA
GCGGATCAGCAATTGCGTGGCCGACTACAGCGTGCTGTACAACAGCGCCAGCTTCAGCA
CCTTCAAGTGCTACGGCGTGTCACCCACCAAGCTGAACGACCTGTGCTTCACCAACGTGT
ACGCCGACAGCTTCGTGATCAGAGGCGACGAAGTGCGGCAGATTGCCCCTGGACAGACA
GGCAAGATCGCCGATTACAACTACAAGCTGCCCGACGACTTCACCGGCTGTGTGATTGCC
TGGAACAGCAACAACCTGGACAGCAAAGTCGGCGGCAACTACAACTACCTGTACCGGCT
GTTCCGGAAGTCCAACCTGAAGCCTTTCGAGCGGGACATCAGCACCGAGATCTATCAGG
CCGGCAGCACCCCTTGCAATGGCGTGGAAGGCTTCAACTGCTACTTCCCACTGCAGTCCT
ACGGCTTCCAGCCTACAAACGGCGTGGGCTACCAGCCTTACAGAGTGGTGGTGCTGAGC
TTCGAGCTGCTGCATGCTCCTGCCACAGTGTGCGGCCCTAAGAAAAGCACCAACCTGGTC
AAGAACAAATGCGTGAACTTCTGA
SEQ ID NO: 4. DNA equivalent of protein coding sequence of SARS-COV-2 S-RBD-
C3d-p28.6 in RNA sequence:
ATGGATGCTATGAAGAGGGGCCTGTGCTGCGTGCTGCTTCTGTGTGGCGCTGTGTTCGT
GTCCCCTAGAGTGCAGCCTACCGAGAGCATCGTGCGGTTCCCCAACATCACCAATCTGTG
CCCTTTCGGCGAGGTGTTCAACGCCACCAGATTCGCCTCTGTGTACGCCTGGAACCGGAA
GCGGATCAGCAATTGCGTGGCCGACTACAGCGTGCTGTACAACAGCGCCAGCTTCAGCA
CCTTCAAGTGCTACGGCGTGTCACCCACCAAGCTGAACGACCTGTGCTTCACCAACGTGT
ACGCCGACAGCTTCGTGATCAGAGGCGACGAAGTGCGGCAGATTGCCCCTGGACAGACA
GGCAAGATCGCCGATTACAACTACAAGCTGCCCGACGACTTCACCGGCTGTGTGATTGCC
TGGAACAGCAACAACCTGGACAGCAAAGTCGGCGGCAACTACAACTACCTGTACCGGCT
GTTCCGGAAGTCCAACCTGAAGCCTTTCGAGCGGGACATCAGCACCGAGATCTATCAGG
CCGGCAGCACCCCTTGCAATGGCGTGGAAGGCTTCAACTGCTACTTCCCACTGCAGTCCT
ACGGCTTCCAGCCTACAAACGGCGTGGGCTACCAGCCTTACAGAGTGGTGGTGCTGAGC
TTCGAGCTGCTGCATGCTCCTGCCACAGTGTGCGGCCCTAAGAAAAGCACCAACCTGGTC
AAGAACAAATGCGTGAACTTCGGCGGAGGCGGAAGTGGTGGCGGCGGATCTAAGTTTCT
GACCACCGCCAAGGACAAGAACAGATGGGAAGATCCCGGCAAGCAGCTGTACAATGTGG
AAGCCACAAGCTACGCAGGCGGCGGAGGAAGCGGAGGCGGAGGTAGTAAATTTCTGAC
AACGGCTAAAGATAAGAATCGCTGGGAAGATCCTGGGAAACAGCTCTATAACGTCGAGG
CCACCAGCTATGCTGGCGGTGGCGGATCTGGCGGCGGTGGTTCAAAATTCCTGACTACA
GCCAAGGATAAGAATCGTTGGGAAGATCCAGGCAAGCAACTCTATAATGTTGAGGCTACC
TCTTACGCTGGTGGCGGAGGTTCTGGCGGCGGAGGCTCTAAATTTCTCACAACAGCAAA
GGACAAGAATCGATGGGAAGATCCGGGAAAACAACTGTACAACGTTGAGGCAACATCCT
ATGCAGGCGGAGGTGGCAGTGGCGGAGGTGGAAGCAAGTTTCTGACTACTGCAAAAGAT
AAGAATAGATGGGAAGATCCCGGGAAGCAACTCTACAACGTCGAAGCTACTAGTTATGCC
GGTGGCGGTGGATCTGGCGGAGGCGGCAGCAAATTCCTGACCACCGCTAAAGACAAGAA
TCGTTGGGAAGATCCCGGAAAGCAGTTGTATAACGTTGAAGCTACGTCCTACGCCTGA
SEQ ID NO: 5. DNA equivalent of protein coding sequence of SARS-Cov-2
nucleocapsid protein (N) in RNA sequence
ATGAGCGACAACGGCCCTCAGAACCAGAGAAACGCCCCTCGGATCACATTTGGCGGCCC
TAGCGATAGCACCGGCAGCAATCAGAATGGCGAGAGAAGCGGCGCCAGAAGCAAGCAG
AGAAGGCCTCAAGGCCTGCCTAACAACACCGCCAGCTGGTTCACAGCCCTGACACAGCA
CGGCAAAGAGGACCTGAAGTTCCCTAGAGGACAGGGCGTGCCCATCAACACCAACAGCA
GCCCCGATGACCAGATCGGCTACTACAGACGGGCCACCAGAAGAATCAGAGGCGGCGAC
GGCAAGATGAAGGATCTGAGCCCCAGATGGTACTTCTACTACCTCGGCACAGGACCCGA
AGCCGGACTTCCTTATGGCGCCAACAAGGACGGCATCATCTGGGTTGCAACAGAAGGCG
CCCTGAACACCCCTAAGGACCACATCGGCACCAGAAATCCCGCCAACAATGCCGCCATTG
TGCTGCAGTTGCCTCAGGGCACAACACTGCCCAAGGGCTTTTACGCCGAGGGCTCTAGA
GGCGGATCTCAGGCCAGCAGCAGAAGCAGCTCCAGATCCAGAAACAGCTCCCGGAATAG
CACCCCTGGCTCCAGCAGAGGAACAAGCCCTGCTAGAATGGCCGGCAACGGCGGAGATG
CTGCTCTGGCACTTCTCCTGCTGGACCGGCTGAATCAGCTGGAAAGCAAGATGAGCGGC
AAGGGACAGCAGCAGCAGGGCCAGACCGTGACAAAAAAGTCTGCCGCCGAGGCCAGCA
AGAAGCCCAGACAGAAAAGAACCGCCACCAAGGCCTACAACGTGACCCAGGCCTTTGGC
AGAAGAGGCCCTGAGCAGACCCAGGGCAATTTCGGCGATCAAGAGCTGATCAGACAGGG
CACCGACTACAAGCACTGGCCTCAGATCGCCCAGTTTGCCCCATCTGCCAGCGCCTTTTT
CGGCATGAGCCGGATCGGCATGGAAGTGACACCTAGCGGCACCTGGCTGACATACACAG
GCGCCATCAAGCTGGACGACAAGGACCCCAACTTCAAGGACCAAGTGATCCTGCTGAAC
AAGCACATCGACGCCTACAAGACATTCCCTCCAACCGAGCCTAAGAAGGACAAGAAGAA
GAAGGCCGACGAGACACAGGCCCTGCCTCAGCGCCAGAAAAAGCAGCAGACAGTGACA
CTGCTGCCAGCCGCCGACCTGGACGATTTTTCTAAGCAGCTGCAGCAGAGCATGAGCAG
CGCCGATTCTACACAGGCCTGA
SEQ ID NO: 6. DNA equivalent of protein coding sequence of SARS-Cov-2
membrane protein (M) in RNA sequence
ATGGCCGATAGCAATGGCACCATCACCGTGGAAGAACTGAAGAAACTGCTGGAACAGTG
GAACCTCGTGATCGGCTTCCTGTTCCTGACCTGGATCTGCCTGCTGCAGTTCGCCTACGC
CAACCGGAACAGATTCCTGTATATTATCAAGCTGATCTTCCTGTGGCTGCTGTGGCCCGT
GACACTGGCCTGTTTTGTGCTGGCCGCCGTGTACCGGATCAACTGGATCACAGGCGGAA
TCGCCATTGCCATGGCCTGTCTCGTTGGCCTGATGTGGCTGAGCTACTTTATCGCCAGCT
TCCGGCTGTTCGCCCGGACCAGATCCATGTGGTCCTTCAATCCCGAGACAAACATCCTGC
TGAACGTGCCCCTGCACGGCACCATCCTTACAAGACCTCTGCTGGAAAGCGAGCTGGTCA
TCGGAGCCGTGATCCTGAGAGGCCACCTGAGAATTGCCGGACACCACCTGGGCAGATGC
GACATCAAGGACCTGCCTAAAGAAATCACAGTGGCCACCAGCAGAACCCTGTCCTACTAT
AAGCTGGGCGCCAGCCAGAGAGTGGCCGGCGATTCTGGATTTGCCGCCTACAGCAGATA
CCGGATCGGCAACTACAAGCTGAACACCGACCACAGCTCCAGCAGCGACAATATCGCAC
TGCTGGTGCAGTGA
SEQ ID NO: 7. DNA equivalent of RNA sequence of non-cytopathic VEE replicon
expressing SARS-COV-2 S-RBD-C3d-p28.6. The sequence encoding S-RBD-C3d-
p28.6 is underlined. Self-replicating RNA molecules of the invention can be
generated by replacing the underlined S-RBD-C3d-p28.6 with the respective SARS-
CoV-2 antigen, such as SEQ ID NO: 3, 5 or 6:
ATGGGCGGCGCATGAGAGAAGCCCAGACCAATTACCTACCCAAAATGGAGAAAGTTCAC
GTTGACATCGAGGAAGACAGCCCATTCCTCAGAGCTTTGCAGCGGAGCTTCCCGCAGTTT
GAGGTAGAAGCCAAGCAGGTCACTGATAATGACCATGCTAATGCCAGAGCGTTTTCGCAT
CTGGCTTCAAAACTGATCGAAACGGAGGTGGACCCATCCGACACGATCCTTGACATTGGA
AGTGCGCCCGCCCGCAGAATGTATTCTAAGCACAAGTATCATTGTATCTGTCCGATGAGA
TGTGCGGAAGATCCGGACAGATTGTATAAGTATGCAACTAAGCTGAAGAAAAACTGTAAG
GAAATAACTGATAAGGAATTGGACAAGAAAATGAAGGAGCTCGCCGCCGTCATGAGCGA
CCCTGACCTGGAAACTGAGACTATGTGCCTCCACGACGACGAGTCGTGTCGCTACGAAG
GGCAAGTCGCTGTTTACCAGGATGTATACGCGGTTGACGGACCGACAAGTCTCTATCACC
AAGCCAATAAGGGAGTTAGAGTCGCCTACTGGATAGGCTTTGACACCACCCCTTTTATGT
TTAAGAACTTGGCTGGAGCATATCCATCATACTCTACCAACTGGGCCGACGAAACCGTGT
TAACGGCTCGTAACATAGGCCTATGCAGCTCTGACGTTATGGAGCGGTCACGTAGAGGG
ATGTCCATTCTTAGAAAGAAGTATTTGAAACCATCCAACAATGTTCTATTCTCTGTTGGCTC
GACCATCTACCACGAGAAGAGGGACTTACTGAGGAGCTGGCACCTGCCGTCTGTATTTCA
CTTACGTGGCAAGCAAAATTACACATGTCGGTGTGAGACTATAGTTAGTTGCGACGGGTA
CGTCGTTAAAAGAATAGCTATCAGTCCAGGCCTGTATGGGAAGCCTTCAGGCTATGCTGC
TACGATGCACCGCGAGGGATTCTTGTGCTGCAAAGTGACAGACACATTGAACGGGGAGA
GGGTCTCTTTTCCCGTGTGCACGTATGTGCCAGCTACATTGTGTGACCAAATGACTGGCA
TACTGGCAACAGATGTCAGTGCGGACGACGCGCAAAAACTGCTGGTTGGGCTCAACCAG
CGTATAGTCGTCAACGGTCGCACCCAGAGAAACACCAATACCATGAAAAATTACCTTTTG
CCCGTAGTGGCCCAGGCATTTGCTAGGTGGGCAAAGGAATATAAGGAAGATCAAGAAGA
TGAAAGGCCACTAGGACTACGAGATAGACAGTTAGTCATGGGGTGTTGTTGGGCTTTTAG
AAGGCACAAGATAACATCTATTTATAAGCGCCCGGATACCCAAACCATCATCAAAGTGAA
CAGCGATTTCCACTCATTCGTGCTGCCCAGGATAGGCAGTAACACATTGGAGATCGGGCT
GAGAACAAGAATCAGGAAAATGTTAGAGGAGCACAAGGAGCCGTCACCTCTCATTACCG
CCGAGGACGTACAAGAAGCTAAGTGCGCAGCCGATGAGGCTAAGGAGGTGCGTGAAGC
CGAGGAGTTGCGCGCAGCTCTACCACCTTTGGCAGCTGATGTTGAGGAGCCCACTCTGG
AAGCCGATGTCGACTTGATGTTACAAGAGGCTGGGGCCGGCTCAGTGGAGACACCTCGT
GGCTTGATAAAGGTTACCAGCTACGATGGCGAGGACAAGATCGGCTCTTACGCTGTGCTT
TCTCCGCAGGCTGTACTCAAGAGTGAAAAATTATCTTGCATCCACCCTCTCGCTGAACAA
GTCATAGTGATAACACACTCTGGCCGAAAAGGGCGTTATGCCGTGGAACCATACCATGGT
AAAGTAGTGGTGCCAGAGGGACATGCAATACCCGTCCAGGACTTTCAAGCTCTGAGTGA
AAGTGCCACCATTGTGTACAACGAACGTGAGTTCGTAAACAGGTACCTGCACCATATTGC
CACACATGGAGGAGCGCTGAACACTGATGAAGAATATTACAAAACTGTCAAGCCCAGCGA
GCACGACGGCGAATACCTGTACGACATCGACAGGAAACAGTGCGTCAAGAAAGAACTAG
TCACTGGGCTAGGGCTCACAGGCGAGCTGGTGGATCCTCCCTTCCATGAATTCGCCTACG
AGAGTCTGAGAACACGACCAGCCGCTCCTTACCAAGTACCAACCATAGGGGTGTATGGC
GTGCCAGGATCAGGCAAGTCTGGCATCATTAAAAGCGCAGTCACCAAAAAAGATCTAGTG
GTGAGCGCCAAGAAAGAAAACTGTGCAGAAATTATAAGGGACGTCAAGAAAATGAAAGG
GCTGGACGTCAATGCCAGAACTGTGGACTCAGTGCTCTTGAATGGATGCAAACACCCCGT
AGAGACCCTGTATATTGACGAAGCTTTTGCTTGTCATGCAGGTACTCTCAGAGCGCTCAT
AGCCATTATAAGACCTAAAAAGGCAGTGCTCTGCGGGGATCCCAAACAGTGCGGTTTTTT
TAACATGATGTGCCTGAAAGTGCATTTTAACCACGAGATTTGCACACAAGTCTTCCACAAA
AGCATCTCTCGCCGTTGCACTAAATCTGTGACTTCGGTCGTCTCAACCTTGTTTTACGACA
AAAAAATGAGAACGACGAATCCGAAAGAGACTAAGATTGTGATTGACACTACCGGCAGTA
CCAAACCTAAGCAGGACGATCTCATTCTCACTTGTTTCAGAGGGTGGGTGAAGCAGTTGC
AAATAGATTACAAAGGCAACGAAATAATGACGGCAGCTGCCTCTCAAGGGCTGACCCGTA
AAGGTGTGTATGCCGTTCGGTACAAGGTGAATGAAAATCCTCTGTACGCACCCACCTCAG
AACATGTGAACGTCCTACTGACCCGCACGGAGGACCGCATCGTGTGGAAAACACTAGCC
GGCGACCCATGGATAAAAACACTGACTGCCAAGTACCCTGGGAATTTCACTGCCACGATA
GAGGAGTGGCAAGCAGAGCATGATGCCATCATGAGGCACATCTTGGAGAGACCGGACCC
TACCGACGTCTTCCAGAATAAGGCAAACGTGTGTTGGGCCAAGGCTTTAGTGCCGGTGCT
GAAGACCGCTGGCATAGACATGACCACTGAACAATGGAACACTGTGGATTATTTTGAAAC
GGACAAAGCTCACTCAGCAGAGATAGTATTGAACCAACTATGCGTGAGGTTCTTTGGACT
CGATCTGGACTCCGGTCTATTTTCTGCACCCACTGTTCCGTTATCCATTAGGAATAATCAC
TGGGATAACTCCCCGTCGCCTAACATGTACGGGCTGAATAAAGAAGTGGTCCGTCAGCTC
TCTCGCAGGTACCCACAACTGCCTCGGGCAGTTGCCACTGGAAGAGTCTATGACATGAAC
ACTGGTACACTGCGCAATTATGATCCGCGCATAAACCTAGTACCTGTAAACAGAAGACTG
CCTCATGCTTTAGTCCTCCACCATAATGAACACCCACAGAGTGACTTTTCTTCATTCGTCA
GCAAATTGAAGGGCAGAACTGTCCTGGTGGTCGGGGAAAAGTTGTCCGTCCCAGGCAAA
ATGGTTGACTGGTTGTCAGACCGGCCTGAGGCTACCTTCAGAGCTCGGCTGGATTTAGG
CATCCCAGGTGATGTGCCCAAATATGACATAATATTTGTTAATGTGAGGACCCCATATAAA
TACCATCACTATCAGCAGTGTGAAGACCATGCCATTAAGCTTAGCATGTTGACCAAGAAA
GCTTGTCTGCATCTGAATCCCGGCGGAACCTGTGTCAGCATAGGTTATGGTTACGCTGAC
AGGGCCAGCGAAAGCATCATTGGTGCTATAGCGCGGCTGTTCAAGTTTTCCCGGGTATGC
AAACCGAAATCCTCACTTGAAGAGACGGAAGTTCTGTTTGTATTCATTGGGTACGATCGC
AAGGCCCGTACGCACAATCCTTACAAGCTTTCATCAACCTTGACCAACATTTATACAGGTT
CCAGACTCCACGAAGCCGGATGTGCACCCTCATATCATGTGGTGCGAGGGGATATTGCC
ACGGCCACCGAAGGAGTGATTATAAATGCTGCTAACAGCAAAGGACAACCTGGCGGAGG
GGTGTGCGGAGCGCTGTATAAGAAATTCCCGGAAAGCTTCGATTTACAGCCGATCGAAGT
AGGAAAAGCGCGACTGGTCAAAGGTGCAGCTAAACATATCATTCATGCCGTAGGACCAAA
CTTCAACAAAGTTTCGGAGGTTGAAGGTGACAAACAGTTGGCAGAGGCTTATGAGTCCAT
CGCTAAGATTGTCAACGATAACAATTACAAGTCAGTAGCGATTCCACTGTTGTCCACCGG
CATCTTTTCCGGGAACAAAGATCGACTAACCCAATCATTGAACCATTTGCTGACAGCTTTA
GACACCACTGATGCAGATGTAGCCATATACTGCAGGGACAAGAAATGGGAAATGACTCTC
AAGGAAGCAGTGGCTAGGAGAGAAGCAGTGGAGGAGATATGCATATCCGACGACTCTTC
AGTGACAGAACCTGATGCAGAGCTGGTGAGGGTGCATCCGAAGAGTTCTTTGGCTGGAA
GGAAGGGCTACAGCACAAGCGATGGCAAAACTTTCTCATATTTGGAAGGGACCAAGTTTC
ACCAGGCGGCCAAGGATATAGCAGAAATTAATGCCATGTGGCCCGTTGCAACGGAGGCC
AATGAGCAGGTATGCATGTATATCCTCGGAGAAAGCATGAGCAGTATTAGGTCGAAATGC
CCCGTCGAAGAGTCGGAAGCCTCCACACCACCTAGCACGCTGCCTTGCTTGTGCATCCAT
GCCATGACTCCAGAAAGAGTACAGCGCCTAAAAGCCTCACGTCCAGAACAAATTACTGTG
TGCTCATCCTTTCCATTGCCGAAGTATAGAATCACTGGTGTGCAGAAGATCCAATGCTCCC
AGCCTATATTGTTCTCACCGAAAGTGCCTGCGTATATTCATCCAAGGAAGTATCTCGTGGA
AACACCACCGGTAGACGAGACTCCGGAGCCATCGGCAGAGAACCAATCCACAGAGGGGA
CACCTGAACAACCACCACTTATAACCGAGGATGAGACCAGGACTAGAACGCCTGAGCCG
ATCATCATCGAAGAGGAAGAAGAGGATAGCATAAGTTTGCTGTCAGATGGCCCGACCCAC
CAGGTGCTGCAAGTCGAGGCAGACATTCACGGGCCGCCCTCTGTATCTAGCTCATCCTG
GTCCATTCCTCATGCATCCGACTTTGATGTGGACAGTTTATCCATACTTGACACCCTGGAG
GGAGCTAGCGTGACCAGCGGGGCAACGTCAGCCGAGACTAACTCTTACTTCGCAAAGAG
TATGGAGTTTCTGGCGCGACCGGTGCCTGCGCCTCGAACAGTATTCAGGAACCCTCCACA
TCCCGCTCCGCGCACAAGAACACCGTCACTTGCACCCAGCAGGGCCTGCTCGAGAACCA
GCCTAGTTTCCACCCCGCCAGGCGTGAATAGGGTGATCACTAGAGAGGAGCTCGAGGCG
CTTACCCCGTCACGCACTCCTAGCAGGTCGGTCTCGAGAACCAGCCTGGTCTCCAACCCG
CCAGGCGTAAATAGGGTGATTACAAGAGAGGAGTTTGAGGCGTTCGTAGCACAACAACA
ATGACGGTTTGATGCGGGTGCATACATCTTTTCCTCCGACACCGGTCAAGGGCATTTACA
ACAAAAATCAGTAAGGCAAACGGTGCTATCCGAAGTGGTGTTGGAGAGGACCGAATTGG
AGATTTCGTATGCCCCGCGCCTCGACCAAGAAAAAGAAGAATTACTACGCAAGAAATTAC
AGTTAAATCCCACACCTGCTAACAGAAGCAGATACCAGTCCAGGAAGGTGGAGAACATGA
AAGCCATAACAGCTAGACGTATTCTGCAAGGCCTAGGGCATTATTTGAAGGCAGAAGGAA
AAGTGGAGTGCTACCGAACCCTGCATCCTGTTCCTTTGTATTCATCTAGTGTGAACCGTGC
CTTTTCAAGCCCCAAGGTCGCAGTGGAAGCCTGTAACGCCATGTTGAAAGAGAACTTTCC
GACTGTGGCTTCTTACTGTATTATTCCAGAGTACGATGCCTATTTGGACATGGTTGACGGA
GCTTCATGCTGCTTAGACACTGCCAGTTTTTGCCCTGCAAAGCTGCGCAGCTTTCCAAAG
AAACACTCCTATTTGGAACCCACAATACGATCGGCAGTGCCTTCAGCGATCCAGAACACG
CTCCAGAACGTCCTGGCAGCTGCCACAAAAAGAAATTGCAATGTCACGCAAATGAGAGAA
TTGCCCGTATTGGATTCGGCGGCCTTTAATGTGGAATGCTTCAAGAAATATGCGTGTAAT
AATGAATATTGGGAAACGTTTAAAGAAAACCCCATCAGGCTTACTGAAGAAAACGTGGTA
AATTACATTACCAAATTAAAAGGACCAAAAGCTGCTGCTCTTTTTGCGAAGACACATAATT
TGAATATGTTGCAGGACATACCAATGGACAGGTTTGTAATGGACTTAAAGAGAGACGTGA
AAGTGACTCCAGGAACAAAACATACTGAAGAACGGCCCAAGGTACAGGTGATCCAGGCT
GCCGATCCGCTAGCAACAGCGTATCTGTGCGGAATCCACCGAGAGCTGGTTAGGAGATT
AAATGCGGTCCTGCTTCCGAACATTCATACACTGTTTGATATGTCGGCTGAAGACTTTGAC
GCTATTATAGCCGAGCACTTCCAGCCTGGGGATTGTGTTCTGGAAACTGACATCGCGTCG
TTTGATAAAAGTGAGGACGACGCCATGGCTCTGACCGCGTTAATGATTCTGGAAGACTTA
GGTGTGGACGCAGAGCTGTTGACGCTGATTGAGGCGGCTTTCGGCGAAATTTCATCAATA
CATTTGCCCACTAAAACTAAATTTAAATTCGGAGCCATGATGAAATCTGGAATGTTCCTCA
CACTGTTTGTGAACACAGTCATTAACATTGTAATCGCAAGCAGAGTGTTGAGAGAACGGC
TAACCGGATCACCATGTGCAGCATTCATTGGAGATGACAATATCGTGAAAGGAGTCAAAT
CGGACAAATTAATGGCAGACAGGTGCGCCACCTGGTTGAATATGGAAGTCAAGATTATAG
ATGCTGTGGTGGGCGAGAAAGCGCCTTATTTCTGTGGAGGGTTTATTTTGTGTGACTCCG
TGACCGGCACAGCGTGCCGTGTGGCAGACCCCCTAAAAAGGCTGTTTAAGCTTGGCAAA
CCTCTGGCAGCAGACGATGAACATGATGATGACAGGAGAAGGGCATTGCATGAAGAGTC
AACACGCTGGAACCGAGTGGGTATTCTTTCAGAGCTGTGCAAGGCAGTAGAATCAAGGTA
TGAAACCGTAGGAACTTCCATCATAGTTATGGCCATGACTACTCTAGCTAGCAGTGTTAAA
TCATTCAGCTACCTGAGAGGGGCCCCTATAACTCTCTACGGCTAACCTGAATGGACTACG
ACATAGTCTAGTCCGCCAAGGCCACCATGGATGCTATGAAGAGGGGCCTGTGCTGCGTG
CTGCTTCTGTGTGGCGCTGTGTTCGTGTCCCCTAGAGTGCAGCCTACCGAGAGCATCGTG
CGGTTCCCCAACATCACCAATCTGTGCCCTTTCGGCGAGGTGTTCAACGCCACCAGATTC
GCCTCTGTGTACGCCTGGAACCGGAAGCGGATCAGCAATTGCGTGGCCGACTACAGCGT
GCTGTACAACAGCGCCAGCTTCAGCACCTTCAAGTGCTACGGCGTGTCACCCACCAAGCT
GAACGACCTGTGCTTCACCAACGTGTACGCCGACAGCTTCGTGATCAGAGGCGACGAAG
TGCGGCAGATTGCCCCTGGACAGACAGGCAAGATCGCCGATTACAACTACAAGCTGCCC
GACGACTTCACCGGCTGTGTGATTGCCTGGAACAGCAACAACCTGGACAGCAAAGTCGG
CGGCAACTACAACTACCTGTACCGGCTGTTCCGGAAGTCCAACCTGAAGCCTTTCGAGCG
GGACATCAGCACCGAGATCTATCAGGCCGGCAGCACCCCTTGCAATGGCGTGGAAGGCT
TCAACTGCTACTTCCCACTGCAGTCCTACGGCTTCCAGCCTACAAACGGCGTGGGCTACC
AGCCTTACAGAGTGGTGGTGCTGAGCTTCGAGCTGCTGCATGCTCCTGCCACAGTGTGC
GGCCCTAAGAAAAGCACCAACCTGGTCAAGAACAAATGCGTGAACTTCGGCGGAGGCGG
AAGTGGTGGCGGCGGATCTAAGTTTCTGACCACCGCCAAGGACAAGAACAGATGGGAAG
ATCCCGGCAAGCAGCTGTACAATGTGGAAGCCACAAGCTACGCAGGCGGCGGAGGAAG
CGGAGGCGGAGGTAGTAAATTTCTGACAACGGCTAAAGATAAGAATCGCTGGGAAGATC
CTGGGAAACAGCTCTATAACGTCGAGGCCACCAGCTATGCTGGCGGTGGCGGATCTGGC
GGCGGTGGTTCAAAATTCCTGACTACAGCCAAGGATAAGAATCGTTGGGAAGATCCAGG
CAAGCAACTCTATAATGTTGAGGCTACCTCTTACGCTGGTGGCGGAGGTTCTGGCGGCG
GAGGCTCTAAATTTCTCACAACAGCAAAGGACAAGAATCGATGGGAAGATCCGGGAAAA
CAACTGTACAACGTTGAGGCAACATCCTATGCAGGCGGAGGTGGCAGTGGCGGAGGTGG
AAGCAAGTTTCTGACTACTGCAAAAGATAAGAATAGATGGGAAGATCCCGGGAAGCAACT
CTACAACGTCGAAGCTACTAGTTATGCCGGTGGCGGTGGATCTGGCGGAGGCGGCAGCA
AATTCCTGACCACCGCTAAAGACAAGAATCGTTGGGAAGATCCCGGAAAGCAGTTGTATA
ACGTTGAAGCTACGTCCTACGCCTGAGGCGCGCCTATGTTACGTGCAAAGGTGATTGTCA
CCCCCCGAAAGACCATATTGTGACACACCCTCAGTATCACGCCCAAACATTTACAGCCGC
GGTGTCAAAAACCGCGTGGACGTGGTTAACATCCCTGCTGGGAGGATCAGCCGTAATTAT
TATAATTGGCTTGGTGCTGGCTACTATTGTGGCCATGTACGTGCTGACCAACCAGAAACA
TAATTGAATACAGCAGCAATTGGCAAGCTGCTTACATAGAACTCGCGGCGATTGGCATGC
CGCCTTAAAATTTTTATTTTATTTTTCTTTTCTTTTCCGAATCGGATTTTGTTTTTAATATTTC
AAAAAAAAAAAAAAAAAAAAAAAAAAAAAAAAAAAAAAAATAGGG
SEQ ID NO: 8. Amino acid sequence of SARS-COV-2 nucleocapsid protein (N), also
present under accession number YP_009724397.2 in genbank:
MSDNGPQNQRNAPRITFGGPSDSTGSNQNGERSGARSKQRRPQGLPNNTASWFTALTQH
GKEDLKFPRGQGVPINTNSSPDDQIGYYRRATRRIRGGDGKMKDLSPRWYFYYLGTGPEAG
LPYGANKDGIIWVATEGALNTPKDHIGTRNPANNAAIVLQLPQGTTLPKGFYAEGSRGGSQA
SSRSSSRSRNSSRNSTPGSSRGTSPARMAGNGGDAALALLLLDRLNQLESKMSGKGQQQQ
GQTVTKKSAAEASKKPRQKRTATKAYNVTQAFGRRGPEQTQGNFGDQELIRQGTDYKHWP
QIAQFAPSASAFFGMSRIGMEVTPSGTWLTYTGAIKLDDKDPNFKDQVILLNKHIDAYKTFPP
TEPKKDKKKKADETQALPQRQKKQQTVTLLPAADLDDFSKQLQQSMSSADSTQA
SEQ ID NO: 9. Amino acid sequence of SARS-COV-2 membrane protein (M), also
present under accession number YP_009724393.1 in genbank:
MADSNGTITVEELKKLLEQWNLVIGFLFLTWICLLQFAYANRNRFLYIIKLIFLWLLWPVTLAC
FVLAAVYRINWITGGIAIAMACLVGLMWLSYFIASFRLFARTRSMWSFNPETNILLNVPLHGTI
LTRPLLESELVIGAVILRGHLRIAGHHLGRCDIKDLPKEITVATSRTLSYYKLGASQRVAGDSG
FAAYSRYRIGNYKLNTDHSSSSDNIALLVQ
SEQ ID NO: 10. DNA equivalent of RNA sequence of non-cytopathic VEE replicon
without antigen ORF:
ATGGGCGGCGCATGAGAGAAGCCCAGACCAATTACCTACCCAAAATGGAGAAAGTTCAC
GTTGACATCGAGGAAGACAGCCCATTCCTCAGAGCTTTGCAGCGGAGCTTCCCGCAGTTT
GAGGTAGAAGCCAAGCAGGTCACTGATAATGACCATGCTAATGCCAGAGCGTTTTCGCAT
CTGGCTTCAAAACTGATCGAAACGGAGGTGGACCCATCCGACACGATCCTTGACATTGGA
AGTGCGCCCGCCCGCAGAATGTATTCTAAGCACAAGTATCATTGTATCTGTCCGATGAGA
TGTGCGGAAGATCCGGACAGATTGTATAAGTATGCAACTAAGCTGAAGAAAAACTGTAAG
GAAATAACTGATAAGGAATTGGACAAGAAAATGAAGGAGCTCGCCGCCGTCATGAGCGA
CCCTGACCTGGAAACTGAGACTATGTGCCTCCACGACGACGAGTCGTGTCGCTACGAAG
GGCAAGTCGCTGTTTACCAGGATGTATACGCGGTTGACGGACCGACAAGTCTCTATCACC
AAGCCAATAAGGGAGTTAGAGTCGCCTACTGGATAGGCTTTGACACCACCCCTTTTATGT
TTAAGAACTTGGCTGGAGCATATCCATCATACTCTACCAACTGGGCCGACGAAACCGTGT
TAACGGCTCGTAACATAGGCCTATGCAGCTCTGACGTTATGGAGCGGTCACGTAGAGGG
ATGTCCATTCTTAGAAAGAAGTATTTGAAACCATCCAACAATGTTCTATTCTCTGTTGGCTC
GACCATCTACCACGAGAAGAGGGACTTACTGAGGAGCTGGCACCTGCCGTCTGTATTTCA
CTTACGTGGCAAGCAAAATTACACATGTCGGTGTGAGACTATAGTTAGTTGCGACGGGTA
CGTCGTTAAAAGAATAGCTATCAGTCCAGGCCTGTATGGGAAGCCTTCAGGCTATGCTGC
TACGATGCACCGCGAGGGATTCTTGTGCTGCAAAGTGACAGACACATTGAACGGGGAGA
GGGTCTCTTTTCCCGTGTGCACGTATGTGCCAGCTACATTGTGTGACCAAATGACTGGCA
TACTGGCAACAGATGTCAGTGCGGACGACGCGCAAAAACTGCTGGTTGGGCTCAACCAG
CGTATAGTCGTCAACGGTCGCACCCAGAGAAACACCAATACCATGAAAAATTACCTTTTG
CCCGTAGTGGCCCAGGCATTTGCTAGGTGGGCAAAGGAATATAAGGAAGATCAAGAAGA
TGAAAGGCCACTAGGACTACGAGATAGACAGTTAGTCATGGGGTGTTGTTGGGCTTTTAG
AAGGCACAAGATAACATCTATTTATAAGCGCCCGGATACCCAAACCATCATCAAAGTGAA
CAGCGATTTCCACTCATTCGTGCTGCCCAGGATAGGCAGTAACACATTGGAGATCGGGCT
GAGAACAAGAATCAGGAAAATGTTAGAGGAGCACAAGGAGCCGTCACCTCTCATTACCG
CCGAGGACGTACAAGAAGCTAAGTGCGCAGCCGATGAGGCTAAGGAGGTGCGTGAAGC
CGAGGAGTTGCGCGCAGCTCTACCACCTTTGGCAGCTGATGTTGAGGAGCCCACTCTGG
AAGCCGATGTCGACTTGATGTTACAAGAGGCTGGGGCCGGCTCAGTGGAGACACCTCGT
GGCTTGATAAAGGTTACCAGCTACGATGGCGAGGACAAGATCGGCTCTTACGCTGTGCTT
TCTCCGCAGGCTGTACTCAAGAGTGAAAAATTATCTTGCATCCACCCTCTCGCTGAACAA
GTCATAGTGATAACACACTCTGGCCGAAAAGGGCGTTATGCCGTGGAACCATACCATGGT
AAAGTAGTGGTGCCAGAGGGACATGCAATACCCGTCCAGGACTTTCAAGCTCTGAGTGA
AAGTGCCACCATTGTGTACAACGAACGTGAGTTCGTAAACAGGTACCTGCACCATATTGC
CACACATGGAGGAGCGCTGAACACTGATGAAGAATATTACAAAACTGTCAAGCCCAGCGA
GCACGACGGCGAATACCTGTACGACATCGACAGGAAACAGTGCGTCAAGAAAGAACTAG
TCACTGGGCTAGGGCTCACAGGCGAGCTGGTGGATCCTCCCTTCCATGAATTCGCCTACG
AGAGTCTGAGAACACGACCAGCCGCTCCTTACCAAGTACCAACCATAGGGGTGTATGGC
GTGCCAGGATCAGGCAAGTCTGGCATCATTAAAAGCGCAGTCACCAAAAAAGATCTAGTG
GTGAGCGCCAAGAAAGAAAACTGTGCAGAAATTATAAGGGACGTCAAGAAAATGAAAGG
GCTGGACGTCAATGCCAGAACTGTGGACTCAGTGCTCTTGAATGGATGCAAACACCCCGT
AGAGACCCTGTATATTGACGAAGCTTTTGCTTGTCATGCAGGTACTCTCAGAGCGCTCAT
AGCCATTATAAGACCTAAAAAGGCAGTGCTCTGCGGGGATCCCAAACAGTGCGGTTTTTT
TAACATGATGTGCCTGAAAGTGCATTTTAACCACGAGATTTGCACACAAGTCTTCCACAAA
AGCATCTCTCGCCGTTGCACTAAATCTGTGACTTCGGTCGTCTCAACCTTGTTTTACGACA
AAAAAATGAGAACGACGAATCCGAAAGAGACTAAGATTGTGATTGACACTACCGGCAGTA
CCAAACCTAAGCAGGACGATCTCATTCTCACTTGTTTCAGAGGGTGGGTGAAGCAGTTGC
AAATAGATTACAAAGGCAACGAAATAATGACGGCAGCTGCCTCTCAAGGGCTGACCCGTA
AAGGTGTGTATGCCGTTCGGTACAAGGTGAATGAAAATCCTCTGTACGCACCCACCTCAG
AACATGTGAACGTCCTACTGACCCGCACGGAGGACCGCATCGTGTGGAAAACACTAGCC
GGCGACCCATGGATAAAAACACTGACTGCCAAGTACCCTGGGAATTTCACTGCCACGATA
GAGGAGTGGCAAGCAGAGCATGATGCCATCATGAGGCACATCTTGGAGAGACCGGACCC
TACCGACGTCTTCCAGAATAAGGCAAACGTGTGTTGGGCCAAGGCTTTAGTGCCGGTGCT
GAAGACCGCTGGCATAGACATGACCACTGAACAATGGAACACTGTGGATTATTTTGAAAC
GGACAAAGCTCACTCAGCAGAGATAGTATTGAACCAACTATGCGTGAGGTTCTTTGGACT
CGATCTGGACTCCGGTCTATTTTCTGCACCCACTGTTCCGTTATCCATTAGGAATAATCAC
TGGGATAACTCCCCGTCGCCTAACATGTACGGGCTGAATAAAGAAGTGGTCCGTCAGCTC
TCTCGCAGGTACCCACAACTGCCTCGGGCAGTTGCCACTGGAAGAGTCTATGACATGAAC
ACTGGTACACTGCGCAATTATGATCCGCGCATAAACCTAGTACCTGTAAACAGAAGACTG
CCTCATGCTTTAGTCCTCCACCATAATGAACACCCACAGAGTGACTTTTCTTCATTCGTCA
GCAAATTGAAGGGCAGAACTGTCCTGGTGGTCGGGGAAAAGTTGTCCGTCCCAGGCAAA
ATGGTTGACTGGTTGTCAGACCGGCCTGAGGCTACCTTCAGAGCTCGGCTGGATTTAGG
CATCCCAGGTGATGTGCCCAAATATGACATAATATTTGTTAATGTGAGGACCCCATATAAA
TACCATCACTATCAGCAGTGTGAAGACCATGCCATTAAGCTTAGCATGTTGACCAAGAAA
GCTTGTCTGCATCTGAATCCCGGCGGAACCTGTGTCAGCATAGGTTATGGTTACGCTGAC
AGGGCCAGCGAAAGCATCATTGGTGCTATAGCGCGGCTGTTCAAGTTTTCCCGGGTATGC
AAACCGAAATCCTCACTTGAAGAGACGGAAGTTCTGTTTGTATTCATTGGGTACGATCGC
AAGGCCCGTACGCACAATCCTTACAAGCTTTCATCAACCTTGACCAACATTTATACAGGTT
CCAGACTCCACGAAGCCGGATGTGCACCCTCATATCATGTGGTGCGAGGGGATATTGCC
ACGGCCACCGAAGGAGTGATTATAAATGCTGCTAACAGCAAAGGACAACCTGGCGGAGG
GGTGTGCGGAGCGCTGTATAAGAAATTCCCGGAAAGCTTCGATTTACAGCCGATCGAAGT
AGGAAAAGCGCGACTGGTCAAAGGTGCAGCTAAACATATCATTCATGCCGTAGGACCAAA
CTTCAACAAAGTTTCGGAGGTTGAAGGTGACAAACAGTTGGCAGAGGCTTATGAGTCCAT
CGCTAAGATTGTCAACGATAACAATTACAAGTCAGTAGCGATTCCACTGTTGTCCACCGG
CATCTTTTCCGGGAACAAAGATCGACTAACCCAATCATTGAACCATTTGCTGACAGCTTTA
GACACCACTGATGCAGATGTAGCCATATACTGCAGGGACAAGAAATGGGAAATGACTCTC
AAGGAAGCAGTGGCTAGGAGAGAAGCAGTGGAGGAGATATGCATATCCGACGACTCTTC
AGTGACAGAACCTGATGCAGAGCTGGTGAGGGTGCATCCGAAGAGTTCTTTGGCTGGAA
GGAAGGGCTACAGCACAAGCGATGGCAAAACTTTCTCATATTTGGAAGGGACCAAGTTTC
ACCAGGCGGCCAAGGATATAGCAGAAATTAATGCCATGTGGCCCGTTGCAACGGAGGCC
AATGAGCAGGTATGCATGTATATCCTCGGAGAAAGCATGAGCAGTATTAGGTCGAAATGC
CCCGTCGAAGAGTCGGAAGCCTCCACACCACCTAGCACGCTGCCTTGCTTGTGCATCCAT
GCCATGACTCCAGAAAGAGTACAGCGCCTAAAAGCCTCACGTCCAGAACAAATTACTGTG
TGCTCATCCTTTCCATTGCCGAAGTATAGAATCACTGGTGTGCAGAAGATCCAATGCTCCC
AGCCTATATTGTTCTCACCGAAAGTGCCTGCGTATATTCATCCAAGGAAGTATCTCGTGGA
AACACCACCGGTAGACGAGACTCCGGAGCCATCGGCAGAGAACCAATCCACAGAGGGGA
CACCTGAACAACCACCACTTATAACCGAGGATGAGACCAGGACTAGAACGCCTGAGCCG
ATCATCATCGAAGAGGAAGAAGAGGATAGCATAAGTTTGCTGTCAGATGGCCCGACCCAC
CAGGTGCTGCAAGTCGAGGCAGACATTCACGGGCCGCCCTCTGTATCTAGCTCATCCTG
GTCCATTCCTCATGCATCCGACTTTGATGTGGACAGTTTATCCATACTTGACACCCTGGAG
GGAGCTAGCGTGACCAGCGGGGCAACGTCAGCCGAGACTAACTCTTACTTCGCAAAGAG
TATGGAGTTTCTGGCGCGACCGGTGCCTGCGCCTCGAACAGTATTCAGGAACCCTCCACA
TCCCGCTCCGCGCACAAGAACACCGTCACTTGCACCCAGCAGGGCCTGCTCGAGAACCA
GCCTAGTTTCCACCCCGCCAGGCGTGAATAGGGTGATCACTAGAGAGGAGCTCGAGGCG
CTTACCCCGTCACGCACTCCTAGCAGGTCGGTCTCGAGAACCAGCCTGGTCTCCAACCCG
CCAGGCGTAAATAGGGTGATTACAAGAGAGGAGTTTGAGGCGTTCGTAGCACAACAACA
ATGACGGTTTGATGCGGGTGCATACATCTTTTCCTCCGACACCGGTCAAGGGCATTTACA
ACAAAAATCAGTAAGGCAAACGGTGCTATCCGAAGTGGTGTTGGAGAGGACCGAATTGG
AGATTTCGTATGCCCCGCGCCTCGACCAAGAAAAAGAAGAATTACTACGCAAGAAATTAC
AGTTAAATCCCACACCTGCTAACAGAAGCAGATACCAGTCCAGGAAGGTGGAGAACATGA
AAGCCATAACAGCTAGACGTATTCTGCAAGGCCTAGGGCATTATTTGAAGGCAGAAGGAA
AAGTGGAGTGCTACCGAACCCTGCATCCTGTTCCTTTGTATTCATCTAGTGTGAACCGTGC
CTTTTCAAGCCCCAAGGTCGCAGTGGAAGCCTGTAACGCCATGTTGAAAGAGAACTTTCC
GACTGTGGCTTCTTACTGTATTATTCCAGAGTACGATGCCTATTTGGACATGGTTGACGGA
GCTTCATGCTGCTTAGACACTGCCAGTTTTTGCCCTGCAAAGCTGCGCAGCTTTCCAAAG
AAACACTCCTATTTGGAACCCACAATACGATCGGCAGTGCCTTCAGCGATCCAGAACACG
CTCCAGAACGTCCTGGCAGCTGCCACAAAAAGAAATTGCAATGTCACGCAAATGAGAGAA
TTGCCCGTATTGGATTCGGCGGCCTTTAATGTGGAATGCTTCAAGAAATATGCGTGTAAT
AATGAATATTGGGAAACGTTTAAAGAAAACCCCATCAGGCTTACTGAAGAAAACGTGGTA
AATTACATTACCAAATTAAAAGGACCAAAAGCTGCTGCTCTTTTTGCGAAGACACATAATT
TGAATATGTTGCAGGACATACCAATGGACAGGTTTGTAATGGACTTAAAGAGAGACGTGA
AAGTGACTCCAGGAACAAAACATACTGAAGAACGGCCCAAGGTACAGGTGATCCAGGCT
GCCGATCCGCTAGCAACAGCGTATCTGTGCGGAATCCACCGAGAGCTGGTTAGGAGATT
AAATGCGGTCCTGCTTCCGAACATTCATACACTGTTTGATATGTCGGCTGAAGACTTTGAC
GCTATTATAGCCGAGCACTTCCAGCCTGGGGATTGTGTTCTGGAAACTGACATCGCGTCG
TTTGATAAAAGTGAGGACGACGCCATGGCTCTGACCGCGTTAATGATTCTGGAAGACTTA
GGTGTGGACGCAGAGCTGTTGACGCTGATTGAGGCGGCTTTCGGCGAAATTTCATCAATA
CATTTGCCCACTAAAACTAAATTTAAATTCGGAGCCATGATGAAATCTGGAATGTTCCTCA
CACTGTTTGTGAACACAGTCATTAACATTGTAATCGCAAGCAGAGTGTTGAGAGAACGGC
TAACCGGATCACCATGTGCAGCATTCATTGGAGATGACAATATCGTGAAAGGAGTCAAAT
CGGACAAATTAATGGCAGACAGGTGCGCCACCTGGTTGAATATGGAAGTCAAGATTATAG
ATGCTGTGGTGGGCGAGAAAGCGCCTTATTTCTGTGGAGGGTTTATTTTGTGTGACTCCG
TGACCGGCACAGCGTGCCGTGTGGCAGACCCCCTAAAAAGGCTGTTTAAGCTTGGCAAA
CCTCTGGCAGCAGACGATGAACATGATGATGACAGGAGAAGGGCATTGCATGAAGAGTC
AACACGCTGGAACCGAGTGGGTATTCTTTCAGAGCTGTGCAAGGCAGTAGAATCAAGGTA
TGAAACCGTAGGAACTTCCATCATAGTTATGGCCATGACTACTCTAGCTAGCAGTGTTAAA
TCATTCAGCTACCTGAGAGGGGCCCCTATAACTCTCTACGGCTAACCTGAATGGACTACG
ACATAGTCTAGTCCGCCAAGGCCACCGGCGCGCCTATGTTACGTGCAAAGGTGATTGTCA
CCCCCCGAAAGACCATATTGTGACACACCCTCAGTATCACGCCCAAACATTTACAGCCGC
GGTGTCAAAAACCGCGTGGACGTGGTTAACATCCCTGCTGGGAGGATCAGCCGTAATTAT
TATAATTGGCTTGGTGCTGGCTACTATTGTGGCCATGTACGTGCTGACCAACCAGAAACA
TAATTGAATACAGCAGCAATTGGCAAGCTGCTTACATAGAACTCGCGGCGATTGGCATGC
CGCCTTAAAATTTTTATTTTATTTTTCTTTTCTTTTCCGAATCGGATTTTGTTTTTAATATTTC
AAAAAAAAAAAAAAAAAAAAAAAAAAAAAAAAAAAAAAAATAGGG
SEQ ID NO: 11. Amino acid sequence of TC-83 nsP1:
MEKVHVDIEEDSPFLRALQRSFPQFEVEAKQVTDNDHANARAFSHLASKLIETEVDPSDTILD
IGSAPARRMYSKHKYHCICPMRCAEDPDRLYKYATKLKKNCKEITDKELDKKMKELAAVMSD
PDLETETMCLHDDESCRYEGQVAVYQDVYAVDGPTSLYHQANKGVRVAYWIGFDTTPFMFK
NLAGAYPSYSTNWADETVLTARNIGLCSSDVMERSRRGMSILRKKYLKPSNNVLFSVGSTIY
HEKRDLLRSWHLPSVFHLRGKQNYTCRCETIVSCDGYVVKRIAISPGLYGKPSGYAATMHRE
GFLCCKVTDTLNGERVSFPVCTYVPATLCDQMTGILATDVSADDAQKLLVGLNQRIVVNGRT
QRNTNTMKNYLLPVVAQAFARWAKEYKEDQEDERPLGLRDRQLVMGCCWAFRRHKITSIYK
RPDTQTIIKVNSDFHSFVLPRIGSNTLEIGLRTRIRKMLEEHKEPSPLITAEDVQEAKCAADEA
KEVREAEELRAALPPLAADVEEPTLEADVDLMLQEAGA
SEQ ID no: 12. Amino acid sequence of TC-83 nsP2:
GSVETPRGLIKVTSYDGEDKIGSYAVLSPQAVLKSEKLSCIHPLAEQVIVITHSGRKGRYAVE
PYHGKVVVPEGHAIPVQDFQALSESATIVYNEREFVNRYLHHIATHGGALNTDEEYYKTVKPS
EHDGEYLYDIDRKQCVKKELVTGLGLTGELVDPPFHEFAYESLRTRPAAPYQVPTIGVYGVPG
SGKSGIIKSAVTKKDLVVSAKKENCAEIIRDVKKMKGLDVNARTVDSVLLNGCKHPVETLYI
DEAFACHAGTLRALIAIIRPKKAVLCGDPKQCGFFNMMCLKVHFNHEICTQVFHKSISRRCTK
SVTSVVSTLFYDKKMRTTNPKETKIVIDTTGSTKPKQDDLILTCFRGWVKQLQIDYKGNEIMT
AAASQGLTRKGVYAVRYKVNENPLYAPTSEHVNVLLTRTEDRIVWKTLAGDPWIKTLTAKYP
GNFTATIEEWQAEHDAIMRHILERPDPTDVFQNKANVCWAKALVPVLKTAGIDMTTEQWNT
VDYFETDKAHSAEIVLNQLCVRFFGLDLDSGLFSAPTVPLSIRNNHWDNSPSPNMYGLNKEV
VRQLSRRYPQLPRAVATGRVYDMNTGTLRNYDPRINLVPVNRRLPHALVLHHNEHPQSDFSS
FVSKLKGRTVLVVGEKLSVPGKMVDWLSDRPEATFRARLDLGIPGDVPKYDIIFVNVRTPYKY
HHYQQCEDHAIKLSMLTKKACLHLNPGGTCVSIGYGYADRASESIIGAIARQFKFSRVCKPK
SSLEETEVLFVFIGYDRKARTHNPYKLSSTLTNIYTGSRLHEAGC
SEQ ID NO: 13. Amino acid sequence of TC-83 nsP3, as it is present in the nsP1-3
precursor:
APSYHVVRGDIATATEGVIINAANSKGQPGGGVCGALYKKFPESFDLQPIEVGKARLVKGAA
KHIIHAVGPNFNKVSEVEGDKQLAEAYESIAKIVNDNNYKSVAIPLLSTGIFSGNKDRLTQSL
NHLLTALDTTDADVAIYCRDKKWEMTLKEAVARREAVEEICISDDSSVTEPDAELVRVHPKS
SLAGRKGYSTSDGKTFSYLEGTKFHQAAKDIAEINAMWPVATEANEQVCMYILGESMSSIRS
KCPVEESEASTPPSTLPCLCIHAMTPERVQRLKASRPEQITVCSSFPLPKYRITGVQKIQCSQP
ILFSPKVPAYIHPRKYLVETPPVDETPEPSAENQSTEGTPEQPPLITEDETRTRTPEPIIIEEEEE
DSISLLSDGPTHQVLQVEADIHGPPSVSSSSWSIPHASDFDVDSLSILDTLEGASVTSGATS
AETNSYFAKSMEFLARPVPAPRTVFRNPPHPAPRTRTPSLAPSRACSRTSLVSTPPGVNRVITR
EELEALTPSRTPSRSVSRTSLVSNPPGVNRVITREEFEAFVAQQQ
SEQ ID NO: 14. Amino acid sequence of TC-83 nsP4:
YIFSSDTGQGHLQQKSVRQTVLSEVVLERTELEISYAPRLDQEKEELLRKKLQLNPTPANRSR
YQSRKVENMKAITARRILQGLGHYLKAEGKVECYRTLHPVPLYSSSVNRAFSSPKVAVEACN
AMLKENFPTVASYCIIPEYDAYLDMVDGASCCLDTASFCPAKLRSFPKKHSYLEPTIRSAVPSA
IQNTLQNVLAAATKRNCNVTQMRELPVLDSAAFNVECFKKYACNNEYWETFKENPIRLTEEN
VVNYITKLKGPKAAALFAKTHNLNMLQDIPMDRFVMDLKRDVKVTPGTKHTEERPKVQVIQA
ADPLATAYLCGIHRELVRRLNAVLLPNIHTLFDMSAEDFDAIIAEHFQPGDCVLETDIASFDKS
EDDAMALTALMILEDLGVDAELLTLIEAAFGEISSIHLPTKTKFKFGAMMKSGMFLTLFVNTVI
NIVIASRVLRERLTGSPCAAFIGDDNIVKGVKSDKLMADRCATWLNMEVKIIDAVVGEKAPY
FCGGFILCDSVTGTACRVADPLKRLFKLGKPLAADDEHDDDRRRALHEESTRWNRVGILSEL
CKAVESRYETVGTSIIVMAMTTLASSVKSFSYLRGAPITLYG
SEQ ID NO: 15. Amino acid sequence of TC-83 nsP2 protein with Q739L mutation:
GSVETPRGLIKVTSYDGEDKIGSYAVLSPQAVLKSEKLSCIHPLAEQVIVITHSGRKGRYAVE
PYHGKVVVPEGHAIPVQDFQALSESATIVYNEREFVNRYLHHIATHGGALNTDEEYYKTVKPS
EHDGEYLYDIDRKQCVKKELVTGLGLTGELVDPPFHEFAYESLRTRPAAPYQVPTIGVYGVPG
SGKSGIIKSAVTKKDLVVSAKKENCAEIIRDVKKMKGLDVNARTVDSVLLNGCKHPVETLYI
DEAFACHAGTLRALIAIIRPKKAVLCGDPKQCGFFNMMCLKVHFNHEICTQVFHKSISRRCTK
SVTSVVSTLFYDKKMRTTNPKETKIVIDTTGSTKPKQDDLILTCFRGWVKQLQIDYKGNEIMT
AAASQGLTRKGVYAVRYKVNENPLYAPTSEHVNVLLTRTEDRIVWKTLAGDPWIKTLTAKYP
GNFTATIEEWQAEHDAIMRHILERPDPTDVFQNKANVCWAKALVPVLKTAGIDMTTEQWNT
VDYFETDKAHSAEIVLNQLCVRFFGLDLDSGLFSAPTVPLSIRNNHWDNSPSPNMYGLNKEV
VRQLSRRYPQLPRAVATGRVYDMNTGTLRNYDPRINLVPVNRRLPHALVLHHNEHPQSDFSS
FVSKLKGRTVLVVGEKLSVPGKMVDWLSDRPEATFRARLDLGIPGDVPKYDIIFVNVRTPYKY
HHYQQCEDHAIKLSMLTKKACLHLNPGGTCVSIGYGYADRASESIIGAIARLFKFSRVCKPKS
SLEETEVLFVFIGYDRKARTHNPYKLSSTLTNIYTGSRLHEAGC
SEQ ID NO: 16. Amino acid sequence of SARS-COV-2 Spike protein:
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQDVNCTEVPVAIHA
DQLTPTWRVYSTGSNVFQTRAGCLIGAEHVNNSYECDIPIGAGICASYQTQTNSPRRARSVA
SQSIIAYTMSLGAENSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNFSQILPDPSKPSKR
SFIEDLLFNKVTLADAGFIKQYGDCLGDIAARDLICAQKFNGLTVLPPLLTDEMIAQYTSALLA
GTITSGWTFGAGAALQIPFAMQMAYRFNGIGVTQNVLYENQKLIANQFNSAIGKIQDSLSST
ASALGKLQDVVNQNAQALNTLVKQLSSNFGAISSVLNDILSRLDKVEAEVQIDRLITGRLQSL
QTYVTQQLIRAAEIRASANLAATKMSECVLGQSKRVDFCGKGYHLMSFPQSAPHGVVFLHVT
YVPAQEKNFTTAPAICHDGKAHFPREGVFVSNGTHWFVTQRNFYEPQIITTDNTFVSGNCDV
VIGIVNNTVYDPLQPELDSFKEELDKYFKNHTSPDVDLGDISGINASVVNIQKEIDRLNEVAK
NLNESLIDLQELGKYEQYIKWPWYIWLGFIAGLIAIVMVTIMLCCMTSCCSCLKGCCSCGSC
CKFDEDDSEPVLKGVKLHYT
SEQ ID NO: 17. DNA equivalent of RNA sequence of SARS-COV-2 Spike protein:
ATGTTCGTGTTCCTGGTGCTGCTGCCTCTGGTGTCCAGCCAGTGTGTGAACCTGACCACC
AGAACACAGCTGCCTCCAGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCCACGTGTCCGGCACCAATGGCACCAAGAGATTCGAC
AACCCCGTGCTGCCCTTCAACGACGGGGTGTACTTTGCCAGCACCGAGAAGTCCAACATC
ATCAGAGGCTGGATCTTCGGCACCACACTGGACAGCAAGACCCAGAGCCTGCTGATCGT
GAACAACGCCACCAACGTGGTCATCAAAGTGTGCGAGTTCCAGTTCTGCAACGACCCCTT
CCTGGGCGTCTACTACCACAAGAACAACAAGAGCTGGATGGAAAGCGAGTTCCGGGTGT
ACAGCAGCGCCAACAACTGCACCTTCGAGTACGTGTCCCAGCCTTTCCTGATGGACCTGG
AAGGCAAGCAGGGCAACTTCAAGAACCTGCGCGAGTTCGTGTTTAAGAACATCGACGGC
TACTTCAAGATCTACAGCAAGCACACCCCTATCAACCTCGTGCGGGATCTGCCTCAGGGC
TTCTCTGCTCTGGAACCCCTGGTGGATCTGCCCATCGGCATCAACATCACCCGGTTTCAG
ACACTGCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTCCTGCTGAAGTA
CAACGAGAACGGCACCATCACCGACGCCGTGGATTGTGCTCTGGATCCTCTGAGCGAGA
CAAAGTGCACCCTGAAGTCCTTCACCGTGGAAAAGGGCATCTACCAGACCAGCAACTTCC
GGGTGCAGCCCACCGAATCCATCGTGCGGTTCCCCAATATCACCAATCTGTGCCCCTTCG
GCGAGGTGTTCAATGCCACCAGATTCGCCTCTGTGTACGCCTGGAACCGGAAGCGGATC
AGCAATTGCGTGGCCGACTACTCCGTGCTGTACAACTCCGCCAGCTTCAGCACCTTCAAG
TGCTACGGCGTGTCCCCTACCAAGCTGAACGACCTGTGCTTCACAAACGTGTACGCCGAC
AGCTTCGTGATCCGGGGAGATGAAGTGCGGCAGATTGCCCCTGGACAGACAGGCAAGAT
CGCCGACTACAACTACAAGCTGCCCGACGACTTCACCGGCTGTGTGATTGCCTGGAACAG
CAACAACCTGGACTCCAAAGTCGGCGGCAACTACAATTACCTGTACCGGCTGTTCCGGAA
GTCCAATCTGAAGCCCTTCGAGCGGGACATCTCCACCGAGATCTATCAGGCCGGCAGCA
CCCCTTGTAACGGCGTGGAAGGCTTCAACTGCTACTTCCCACTGCAGTCCTACGGCTTTC
AGCCCACAAATGGCGTGGGCTATCAGCCCTACAGAGTGGTGGTGCTGAGCTTCGAACTG
CTGCATGCCCCTGCCACAGTGTGCGGCCCTAAGAAAAGCACCAATCTCGTGAAGAACAAA
TGCGTGAACTTCAACTTCAACGGCCTGACCGGCACCGGCGTGCTGACAGAGAGCAACAA
GAAGTTCCTGCCATTCCAGCAGTTTGGCCGGGATATCGCCGATACCACAGACGCCGTTAG
AGATCCCCAGACACTGGAAATCCTGGACATCACCCCTTGCAGCTTCGGCGGAGTGTCTGT
GATCACCCCTGGCACCAACACCAGCAATCAGGTGGCAGTGCTGTACCAGGACGTGAACT
GTACCGAAGTGCCCGTGGCCATTCACGCCGATCAGCTGACACCTACATGGCGGGTGTAC
TCCACCGGCAGCAATGTGTTTCAGACCAGAGCCGGCTGTCTGATCGGAGCCGAGCACGT
GAACAATAGCTACGAGTGCGACATCCCCATCGGCGCTGGCATCTGTGCCAGCTACCAGA
CACAGACAAACAGCCCCAGACGGGCCAGATCTGTGGCCAGCCAGAGCATCATTGCCTAC
ACAATGTCTCTGGGCGCCGAGAACAGCGTGGCCTACTCCAACAACTCTATCGCTATCCCC
ACCAACTTCACCATCAGCGTGACCACAGAGATCCTGCCTGTGTCCATGACCAAGACCAGC
GTGGACTGCACCATGTACATCTGCGGCGATTCCACCGAGTGCTCCAACCTGCTGCTGCAG
TACGGCAGCTTCTGCACCCAGCTGAATAGAGCCCTGACAGGGATCGCCGTGGAACAGGA
CAAGAACACCCAAGAGGTGTTCGCCCAAGTGAAGCAGATCTACAAGACCCCTCCTATCAA
GGACTTCGGCGGCTTCAATTTCAGCCAGATTCTGCCCGATCCTAGCAAGCCCAGCAAGCG
GAGCTTCATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAGAAGT
TTAACGGACTGACAGTGCTGCCTCCTCTGCTGACCGATGAGATGATCGCCCAGTACACAT
CTGCCCTGCTGGCCGGCACAATCACAAGCGGCTGGACATTTGGAGCTGGCGCCGCTCTG
CAGATCCCCTTTGCTATGCAGATGGCCTACCGGTTCAACGGCATCGGAGTGACCCAGAAT
GTGCTGTACGAGAACCAGAAGCTGATCGCCAACCAGTTCAACAGCGCCATCGGCAAGAT
CCAGGACAGCCTGAGCAGCACAGCAAGCGCCCTGGGAAAGCTGCAGGACGTGGTCAAC
CAGAATGCCCAGGCACTGAACACCCTGGTCAAGCAGCTGTCCTCCAACTTCGGCGCCATC
AGCTCTGTGCTGAACGATATCCTGAGCAGACTGGACAAGGTGGAGGCCGAGGTGCAGAT
CGACAGACTGATCACAGGCAGACTGCAGAGCCTCCAGACATACGTGACCCAGCAGCTGA
TCAGAGCCGCCGAGATTAGAGCCTCTGCCAATCTGGCCGCCACCAAGATGTCTGAGTGT
GTGCTGGGCCAGAGCAAGAGAGTGGACTTTTGCGGCAAGGGCTACCACCTGATGAGCTT
CCCTCAGTCTGCCCCTCACGGCGTGGTGTTTCTGCACGTGACATATGTGCCCGCTCAAGA
GAAGAATTTCACCACCGCTCCAGCCATCTGCCACGACGGCAAAGCCCACTTTCCTAGAGA
AGGCGTGTTCGTGTCCAACGGCACCCATTGGTTCGTGACACAGCGGAACTTCTACGAGC
CCCAGATCATCACCACCGACAACACCTTCGTGTCTGGCAACTGCGACGTCGTGATCGGCA
TTGTGAACAATACCGTGTACGACCCTCTGCAGCCCGAGCTGGACAGCTTCAAAGAGGAAC
TGGACAAGTACTTTAAGAACCACACAAGCCCCGACGTGGACCTGGGCGATATCAGCGGA
ATCAATGCCAGCGTCGTGAACATCCAGAAAGAGATCGACCGGCTGAACGAGGTGGCCAA
GAATCTGAACGAGAGCCTGATCGACCTGCAAGAACTGGGGAAGTACGAGCAGTACATCA
AGTGGCCCTGGTACATCTGGCTGGGCTTTATCGCCGGACTGATTGCCATCGTGATGGTCA
CAATCATGCTGTGTTGCATGACCAGCTGCTGTAGCTGCCTGAAGGGCTGTTGTAGCTGTG
GCAGCTGCTGCAAGTTCGACGAGGACGATTCTGAGCCCGTGCTGAAGGGCGTGAAACTG
CACTACACCTGA
SEQ ID NO: 18. Amino acid sequence of TC-83 nsP3 as it is present in the nsP1-4
precursor:
APSYHVVRGDIATATEGVIINAANSKGQPGGGVCGALYKKFPESFDLQPIEVGKARLVKGAA
KHIIHAVGPNFNKVSEVEGDKQLAEAYESIAKIVNDNNYKSVAIPLLSTGIFSGNKDRLTQSL
NHLLTALDTTDADVAIYCRDKKWEMTLKEAVARREAVEEICISDDSSVTEPDAELVRVHPKS
SLAGRKGYSTSDGKTFSYLEGTKFHQAAKDIAEINAMWPVATEANEQVCMYILGESMSSIRS
KCPVEESEASTPPSTLPCLCIHAMTPERVQRLKASRPEQITVCSSFPLPKYRITGVQKIQCSQP
ILFSPKVPAYIHPRKYLVETPPVDETPEPSAENQSTEGTPEQPPLITEDETRTRTPEPIIIEEEEE
DSISLLSDGPTHQVLQVEADIHGPPSVSSSSWSIPHASDFDVDSLSILDTLEGASVTSGATS
AETNSYFAKSMEFLARPVPAPRTVFRNPPHPAPRTRTPSLAPSRACSRTSLVSTPPGVNRVITR
EELEALTPSRTPSRSVSRTSLVSNPPGVNRVITREEFEAFVAQQQRFDAGA
SEQ ID NO: 19. Amino acid sequence of the human tissue plasminogen activator
leader peptide:
MDAMKRGLCCVLLLCGAVFVSP

### EXAMPLES

### Example 1—Expression of Vaccine Antigen by Sa-RNA

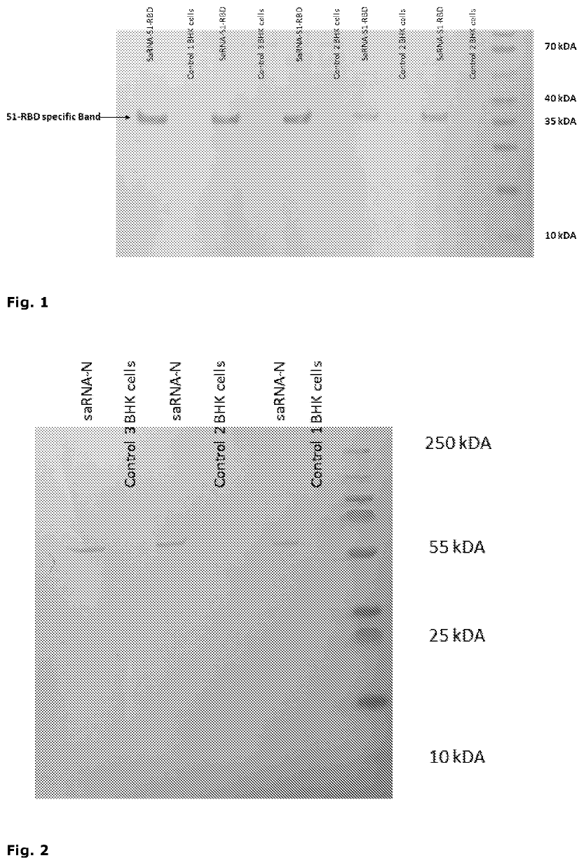

The Receptor Binding domain of the Spike protein (S-RBD, SEQ ID NO: 3) and Nucleocapsid (N, SEQ ID NO: 5) of SARS-CoV-2 virus was cloned in a self-amplifying RNA (saRNA) molecule derived from the TC-83 vaccine strain of Venezuelan Equine Encephalitis virus containing both an A3G substitution in the 5′UTR and an nsP2 Q739L mutation. The saRNA-S-RBD and saRNA-N was transfected in Baby Hamster Kidney 21 (BHK-21) cells. Total protein was isolated from BHK cells transfected with either saRNA expressing the SARS-CoV-2 S-RBD or N antigen or non-transfected control cells and used to perform Western blot. In FIG. 1:Baby Hamster Kidney (BHK)-21 cells (50,000) transfected with 1 μg of saRNA-S1RBD, the expression of SARS-COV-2-S1 RBD protein (˜35 kDa) in the cells was detected by Western blot (indicated by band) using commercially available specific polyclonal antibody (RayBiotech, code: 130-10759). Non transfected BHK cells were also tested as control, where no specific band was identified. The result indicates specificity of expression of the encoded protein by saRNA-S1RBD.

In FIG. 2:

Baby Hamster Kidney (BHK)-21 cells (50,000) when transfected with 1 μg of saRNA-N protein, the expression of SARS-COV-2-N protein (˜55 kDa) in the cells was detected by Western blot (indicated by band) using commercially available specific polyclonal antibody (RayBiotech, Code: 130-10760). Non transfected BHK cells were also tested as control, where no specific band was identified. The result indicates specificity of expression of the encoded protein by saRNA-N.

### Example 2—Vaccine Specific Adaptive Immune Responses

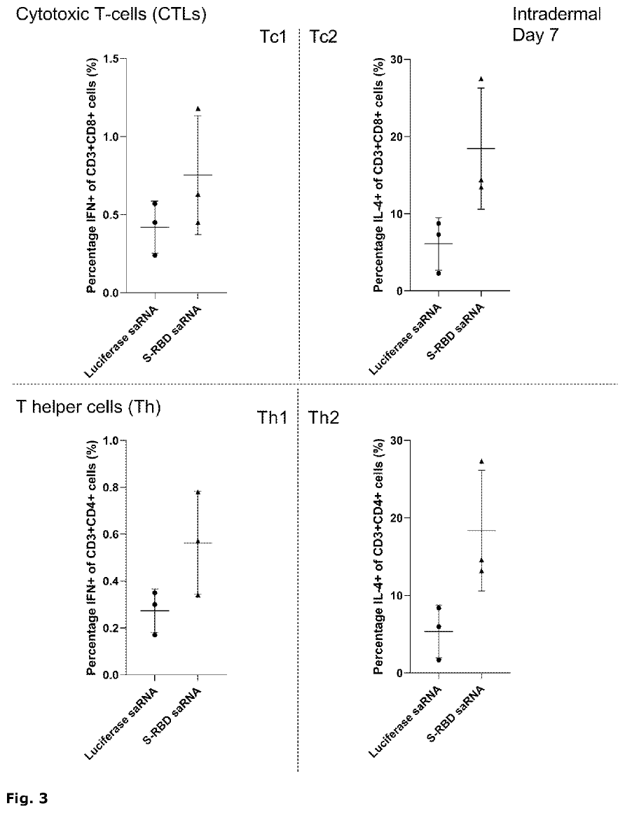

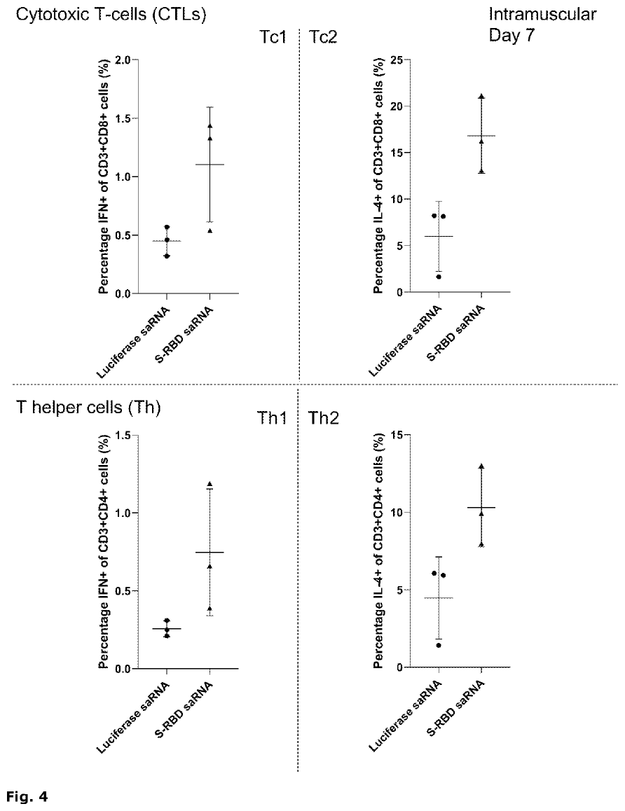

saRNA-S-RBD was delivered to SWISS mice via either intramuscular injection (IM) or intradermal electroporation (ID). saRNA expressing luciferase was delivered in an identical manner and was used as a control. Spleens of both vaccinated and mock control mouse (n=3) were isolated 7 days after injection. Splenocytes were stimulated with a SARS CoV-2 S peptide cocktail to evaluate the responses of Cytotoxic T cells (CD3+CD8+) and T helper cells (CD3+CD4+). A high percentage (3 time more than the mock control) of IL-4+ (humoral response, Th2/Tc2) cells were observed in both Cytotoxic T cells and T helper cells of saRNA-S-RBD treated mice (Both IM and ID). INF+ (Th1/Tc1, cellular immunity) cells also showed elevated level in the treated mice group compared to saRNA-luciferase group (FIGS. 3 and 4).

Intradermal Injection

SWISS outbred mice were intradermally injected with 1 μg saRNA-S1 RBD followed by electroporation on Day 0. Splenocytes were isolated on Day 7 and checked for T cell specific cytotoxic (CD8+) and humoral responses (CD4+) using Flow cytometry. All samples were stimulated for SARS-CoV-2 S protein coding Peptide cocktail. Luc-saRNA injected mice were used as control for comparison. The elevation in IL-4 and IFN-g CD4+ cells, and elevation of CD8+ cells both indicate the induction of helper and cytotoxic adaptive T cell-mediated responses. Results are shown in FIG. 3.

Intramuscular Injection

SWISS outbred mice were intramuscularly injected with 1 μg saRNA-S1 RBD on Day 0. Splenocytes were isolated on Day 7 and checked for T cell specific cytotoxic (CD8+) and humoral responses (CD4+) using Flow cytometry. All samples were stimulated for SARS-CoV-2 S protein coding Peptide cocktail. Luc-saRNA injected mice were used as control for comparison. The elevation in IL-4 and IFN-g CD4+ cells, and elevation of CD8+ cells both indicate the induction of helper and cytotoxic adaptive T cell-mediated responses. Results are shown in FIG. 4.

### Example 3—Vaccine Antigen Expression of LNP Formulated Sa-RNA

In order to enhance stability and intracellular delivery, the SARS-CoV-2 antigen saRNA constructs were formulated in Lipid Nanoparticles (LNPs).

Briefly, LNPs containing encapsulated saRNA were prepared on the Ignite™ System (Precision Nanosytems, PNI). The lipid solution (complexing lipid C12-200, cholesterol, DOPE, DMG-PEG 2000) was prepared by dissolving the lipids in 100% ethanol. LNPs were prepared by loading the lipid solution into the Ignite™ apparatus in combination with in vitro transcribed saRNA constructs in a 3:1 ratio in citrate buffer. A total of 1 μg saRNA was used to prepare the LNP formulations which were diluted to a total volume of 40 μl with PBS. For the combination vaccine, LNP formulations comprising the S-RBD saRNA were mixed in a 1:1 ratio with LNP formulations comprising the N saRNA (0.5 μg saRNA-S-RBD+0.5 μg saRNA-N). A luciferase-encoding saRNA (mock) LNP formulation was also prepared in the same conditions.

In Vivo Expression of the saRNA LNP Formulations

A luciferase encoding saRNA (mock) LNP formulation was prepared as described above and used to assess in vivo expression of the saRNA platform in SWISS mice. Female SWISS mice (6-8 weeks old) were obtained from Janvier (France) and kept in individually ventilated cages with ad libitum access to food and water. Mice were anesthetized using isoflurane. One microgram of luciferase-encoding saRNA (mock) LNP formulation was injected in the gastrocnemius muscle of six mice. Mice were immunized according to a prime-boost vaccination regime with a 21-day interval. Total flux luciferase-induced bioluminescence was determined by non-invasive in vivo imaging system (IVIS) Lumia III scanning 12 minutes after subcutaneous D-luciferin injection, at several time points of the vaccination regime. The results of this assessment are shown in FIG. 5.

Following the first administration of the mock vaccine (Day 0, baseline), bioluminescence signal increased rapidly (from approx. 2.0×10⁴ p/s to 1×10⁸ p/s) and remained high before slowly decreasing ten days after injection. Similar rapid onset of bioluminescent signals was detected after the second administration (boost). By contrast, using similar experimental conditions, Pardi et al., 2015 observed the induction of a bioluminescence signal 7 days post-inoculation ten times lower (approx. 2.0×10⁶ p/s) than the one reported herein. Similarly, de Alwis et al., 2020 reported that 2 μg of saRNA were needed to obtain bioluminescence signals as those reported herein, using the Arcturus platform.

These data confirm that the saRNA platform developed herein is able to yield prolonged antigen expression when administered to SWISS mice.

### Example 4—Binding Antibody Responses in Mice Following Prime Vaccination

SARS-CoV-2 specific humoral responses following intramuscular immunization with a prime injection of 1 μg of saRNA LNP formulations were characterized via serum collected on day 21 of female SWISS mice using ELISA (n=6).

In vivo antigen expression was performed as described in Example 3 above. Blood samples were collected from the tail vein prior to prime injection (day 0), prior to boost injection (day 21) and at sacrifice (day 35). Mice were euthanized via cervical dislocation, and bronchial alveolar lavage (BAL) fluids and spleens were harvested. Serum was collected post blood coagulation by centrifugation and aliquoted for storage at −80° C. until further use.

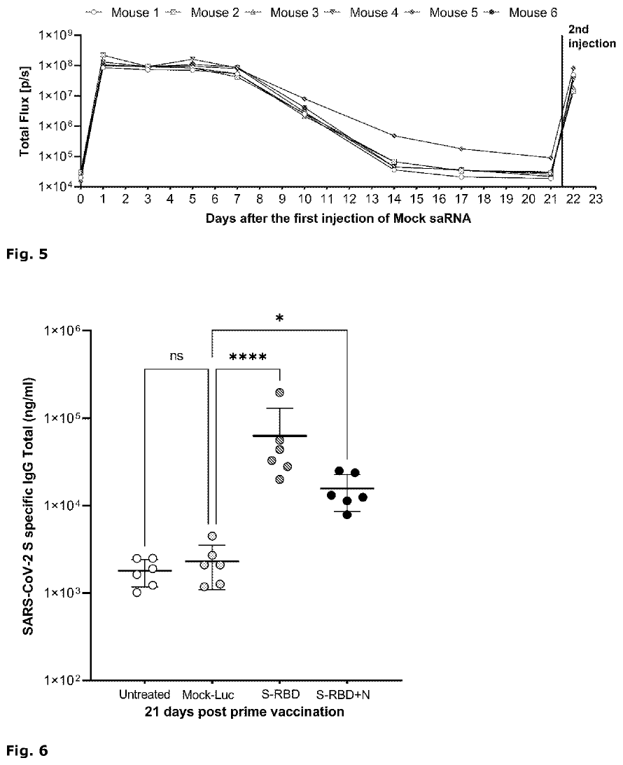

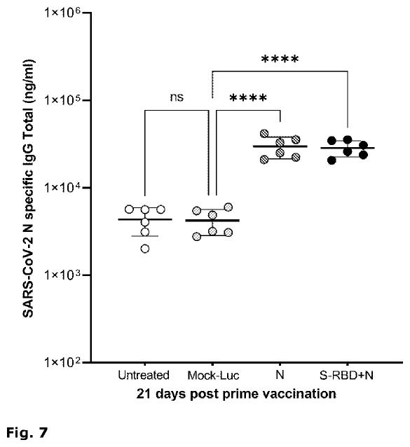

Antigen-specific total IgG titers in mice sera were assessed using Corning™ Costar™ enzyme-linked immune-sorbent assay (ELISA) plates coated with 1 μg/mL recombinant SARS-CoV-2 51 subunit protein RBD (S-RBD), recombinant SARS-CoV-2 Nucleocapsid protein (N). ELISA plates were blocked, incubated with prediluted serum samples and HRP-conjugated detection antibodies were added for total IgG titer. Plates were washed, developed using TMB substrate solution and read on a spectrophotometer at 450 nm (570 nm background). The results of this assessment are shown in FIGS. 6 and 7.

Immunizations with LNP formulated S-RBD antigen (saRNA-S-RBD) and N antigen (saRNA-N) as separate vaccines induced 100% seroconversion three weeks after prime vaccination with significant robust total S antigen-binding IgG antibody response (6.29×10⁴, p<0.0001, FIG. 6) as well as N antigen-binding IgG antibody response (2.97×10⁴, p<0.05) compared to mock vaccination (FIG. 7). In addition, combined vaccination (saRNA-S-RBD+saRNA-N) also induced elevated levels of S and N antigen-specific binding IgG titers three weeks after prime immunization (1.56×10⁴, p<0.0001 and 2.85×10⁴, p<0.0001, respectively) as shown in FIGS. 6 and 7, respectively.

These data show that prime vaccination with LNP formulated saRNA-S-RBD and saRNA-N induces robust binding antibody responses in SWISS mice, both in individual and combined vaccination regimes.

### Example 5—Binding Antibody Responses in Mice Following Boost Vaccination

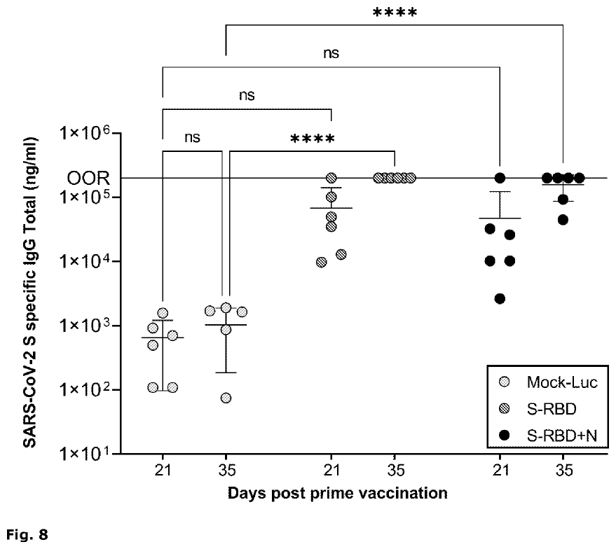

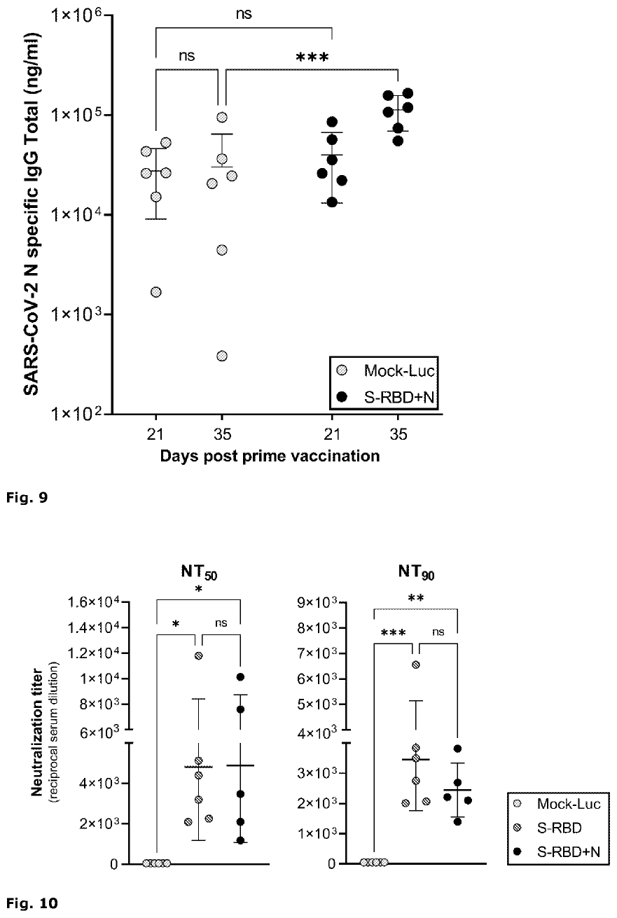

SARS-CoV-2 specific humoral responses following intramuscular immunization with a boost injection of 1 μg of saRNA LNP formulations were characterized using ELISA via serum of SWISS mice collected two weeks after boost vaccination (i.e. five weeks post-prime vaccination, day 35 from first immunization). Experimental methods were similar as described above for Example 4. The results of these assessments are shown in FIGS. 8 and 9.

Mean S-specific IgG titers from samples collected 21 days post prime injection from saRNA-S-RBD vaccinated mice as well as mice vaccinated with the combination of saRNA-S-RBD and saRNA-N remained equally elevated (6.55×10⁴ and 3.13×10⁴, respectively) as shown in FIG. 8. Similar results were obtained for mean N-specific IgG titers from mice vaccinated with the combination of saRNA-S-RBD and saRNA-N (3.42×10⁴) as shown in FIG. 9.

In samples collected 35 days post prime injection (i.e. two weeks after boost vaccination), both S-specific and N-specific IgG titers statistical significance was lost in the group vaccinated with the combination of saRNA-S-RBD and saRNA-N. This is due to the fact that many samples collected on day 35 post prime injection contained IgG titers that were too high to be measured using regular sample dilutions (IgG titers >2×10⁵).

These experiments demonstrate that prime vaccination with individual and combined saRNA LNP formulations induces important SARS-CoV-2 binding antibody responses, which can be enhanced after boost immunization. The S-specific IgG titers obtained using the saRNA platform described herein over-perform as compared to published data from other SARS-CoV vaccine developers. Indeed, according to Vogel et al. 2020, prime only vaccination of BALB/c mice with 1 μg of conventional mRNA vaccine resulted in <10² S-specific binding IgG titers. Prime and boost vaccination of non-human primers (NHP) with 30 μg of the same vaccine was only able to induce S-specific binding IgG titers up to approx. 1.5×10⁴. On the other hand, prime and boost vaccination of three different mice strains with 1 μg of the mRNA vaccine described in Corbett et al., 2020 was able to induce higher S-specific IgG titers, reaching levels ranging between 10⁵ and 10⁶. Using a different saRNA platform than the one described herein, de Alwis et al., 2020 reported S-specific IgG levels of about 10⁶ 30 days post-prime vaccination of BALB/c and C57BL/6 mice using 2 μg of their saRNA constructs. Similarly, two different groups reported S-specific IgG titers after prime and boost vaccination of BALB/c mice with 1 μg of alternative saRNA constructs of approx. 10⁶ (McKay et al. 2020) and <10³ (Erasmus et al. 2020). Finally, one group was able to induce S-specific IgG titers as high as 1.3×10⁵ until 12 weeks after prime only vaccination of BALB/c mice, but 10 μg of saRNA were needed to obtain such results.

### Example 6— Immunization with LNP Formulated Sa-RNA-S-RBD Neutralizes In Vitro Wild-Type SARS-CoV-2 Infection in Mice

In order to evaluate the capacity of specific SARS-CoV-2 antibodies generated by boost immunization to neutralize wild-type SARS-CoV-2 virus infection, a wild-type virus neutralization test (wtVNT) was performed (with Wuhan SARS-CoV-2 strain). Serum dilutions in medium were incubated with 3×TCID100 of SARS-CoV-2 and sample-virus mixtures were added to a cell suspension containing Vero cells 96-well plates. After a 5-day incubation period, the cytopathic effect (CPE) of each well was evaluated and scored microscopically as negative or positive for viral growth. The Reed-Muench method was used to calculate the neutralization titer (NT) which reduces the number of infected cells by 50% (NT₅₀) or 90% (NT₉₀).

While neutralizing antibodies could be observed in mice vaccinated with the LNP formulated sa-RNA-N alone, only NT₅₀ titers similar to control mice (>50) were reached in these groups. In contrast, highly efficient viral neutralization was observed for mice vaccinated with the LNP formulated sa-RNA-S-RBD and combined sa-RNA-S-RBD+sa-RNA-N, with mean NT₅₀ titers reaching 4.8×10³ and 4.9×10³ respectively. Mean NT₉₀ titers reached values of 3.5×10³ and 2.4×10³ for each vaccinated group, respectively. The results of this assessment is shown in FIG. 10.

Care should be taken in the interpretation of the results described above. Indeed, a wrong interpretation could suggest that the addition of the LNP formulated sa-RNA-N to the vaccine formulation (in addition to the LNP formulated sa-RNA-S-RBD) does not provide any benefit in terms of protection against SARS-CoV-2, as NT₅₀ and NT₉₀ titers do not significantly differ between the S-RBA and the S-RBD+N vaccine groups. In fact, this observation results from a limitation in the experimental design of the witl-type VNT assay. In such assay, serum dilutions from vaccinated mice are incubated with SARS-CoV-2 for an amount of time, before this mixture is added to a 96-well plate seeded with cells to be incubated for 5 days. Afterwards, the sample dilution that was able to reduce the number of infected wells by 50% (NT₅₀) or 90% (NT₉₀) is calculated. The serum samples contain both S-specific and N-specific binding antibodies, as observed by ELISA (see FIGS. 6-9). However, only S-specific antibodies are able to bind (and thus neutralize) the SARS-CoV-2, as the S-antigen is located at the surface of the viral particle while the N-antigen is hidden in the internal nucleocapsid. Hence, it would be practically impossible to reach higher NT₅₀/NT₉₀ levels with an S-RBD+N saRNA vaccine versus an S-RBD saRNA vaccine.

In addition, is has been suggested that the cellular incorporation of two saRNA constructs might lead to replicative competition, as one construct outcompetes the other due to preferential replication of the replicase complex Wroblewska et al., 2015). This might disturb or impede the individual immunologic effects of the antigens, as one of both antigens will be less expressed. Hence, the fact that the S-RBD+N saRNA vaccine candidates is able to induce neutralizing antibody responses that are equally high (and not less, as there are no reciprocal significant differences) as the S-RBD saRNA vaccine (FIG. 10), is very promising.

In contrast, none of the published data from either conventional RNA or saRNA manufacturers were able to reach NT₅₀ titers as high as the ones disclosed herein for the combined vaccine candidate (4.9×10³). Specifically, prime only vaccination of BALB/c mice with 1 μg of the vaccine described in Vogel et al., 2020 induced NT₅₀ specific titers lower than 10². Similarly, prime and boost vaccination with 30 μg of the same vaccine in NHP was only able to induce NT₅₀ titers of up to approx. 9.62 x 10². On the other hand, 1 μg of the mRNA construct described in Corbett et al., 2020 induced NT₅₀ levels between 89 and 1119 after prime and boost vaccination of 3 various mouse strains.

Similar results were obtained with various other sa-RNA vaccine candidates. De Alwis et al., 2020 reported NT₅₀ titers of 320 for prime vaccination with 2 μg saRNA (wtVNT). Gritstone Oncology on the other hand reported NT₅₀ titers of 1910 for prime vaccination with 10 μg saRNA (see https://ir.gritstoneoncology.com/static-files/6a7c26ca-06a6-4295-bf76-83948a341397). Also for prime-boost vaccination with 1 μg sa-RNA McKay et al., 2020 and Erasmus et al., 2020 reported NT₅₀ titers of 2560 and 320, respectively.

### Example 7—Combined Immunization with LNP Formulated Sa-RNA-S-RBD and Sa-RNA-N Elicits Protection Against In Vivo Wild-Type SARS-CoV-2 Infection in Hamster

The in vivo efficacy of combined vaccination with sa-RNA-S-RBD+sa-RNA-N against SARS-CoV-2 was evaluated in an intranasal SG hamster challenge experiment. The experiments were performed as described in Sanchez-Felipe et al., 2021. Briefly, six to eight weeks old female SG hamsters (90-120 g body weight) were ear-tagged and randomized in different treatment groups, while housed in individually ventilated cages. At day 0, hamsters were bled in order to determine antigen-specific binding (IIFA; total IgG) and neutralizing (pseudo-typed virus serum neutralization test—psVNT) antibody titers. Each animal also received an intramuscular (thigh muscle) injection (100 μl in each leg) of combined vaccine comprising LNP formulated sa-RNA-S-RBD+sa-RNA-N(as described above) at three different doses (0.1 μg, 1 μg and 5 μg). Control groups were injected with the same amounts of luciferase-saRNA or sham PBS/LNP diluent. Six animals were treated for each experimental condition, for a total of 30 animals.

On day 21, blood was collected again (prior to boost) and hamsters received a second IM (thigh muscle) injection (booster) with the combined vaccine at three different doses or luciferase saRNA or sham/PBS/LNP diluent (n=6 per condition). On day 35, blood was sampled and hamsters were intranasally infected with SARS-CoV-2 (Wuhan SARS-CoV-2 strain, passage 3 at 10E4 TCID50/m1 of VeroE6-grown BetaCoV/Belgium/GHB-03021/2020). In brief, hamsters were anaesthetized by intraperitoneal injection of a xylazine, ketamine and atropine solution. Each hamster was inoculated intranasally by adding 50 μl droplets of virus stock in both nostrils. In practice, & 150 cm² Vero cell culture flask was infected with a previous low passage SARS-CoV-2 stock [BetaCoV/Belgium/GHB-03021/2020] at a 1/1000 final dilution. On day 3, post-infection upon CPE, the virus-containing supernatants were harvested, aliquoted and stored at −80° C. The infectious virus load was determined by plaque assay.

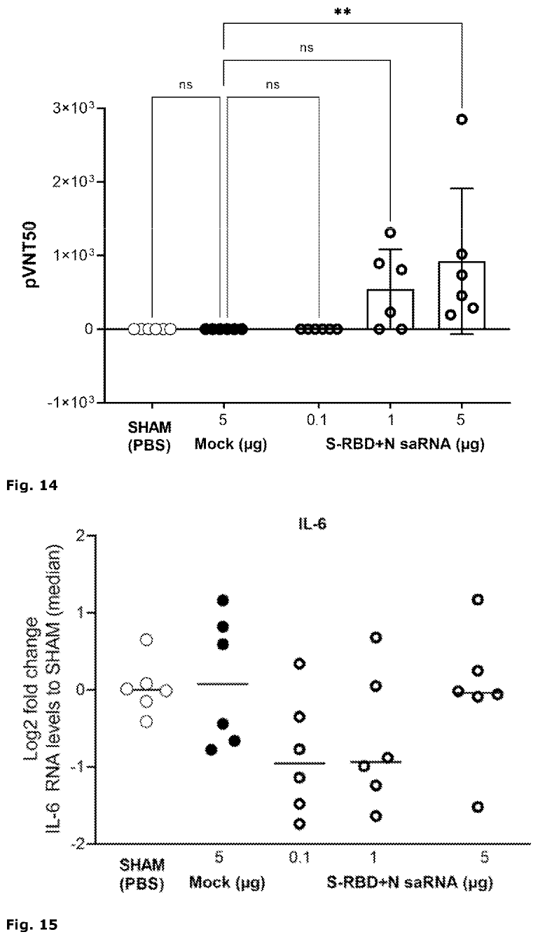

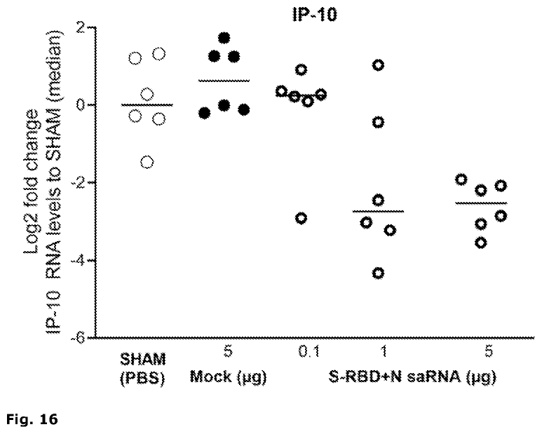

Also on day 35 and prior to challenge, efficient pseudo-viral neutralization (Wuhan SARS-CoV-2 strain) was observed for hamsters vaccinated with 1 μg and 5 μg S-RBD+N saRN, with mean NT₅₀ neutralization titers reaching 5.4×10² and 9.2×10², respectively. The results are depicted in FIG. 14.

After challenge, hamsters were weighted daily and monitored daily for signs of disease (lethargy, heavy breathing or ruffled fur), mobility, self-maintenance, and humane endpoint (hind limb paralysis, hunchback, souring of eyes).

On day 39, 4 days after challenge, animals were euthanized by intraperitoneal administration of 500 μl Dolethal (200 mg/ml sodium pentobarbital) to collect sera and lungs. Lungs were harvested for (i) quantification of viral load by real-time quantitative PCR (RT-qPCR), (ii) quantification of infectious viral content by filtration, (iii) histological examination, and (iv) cytokine analysis (IL-6, IP-10) in the lungs. Blood was collected and serum stored for further analysis.

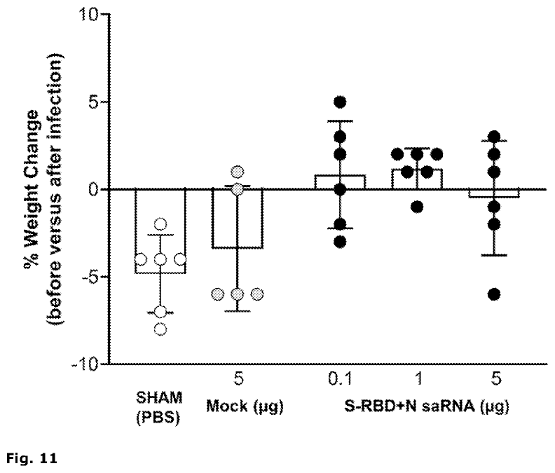

Five weeks after prime administration of the treatments (i.e. two weeks after boost), hamsters were intranasally infected with wild-type SARS-CoV-2 (Wuhan strain). After challenge, hamsters were weighed daily until sacrifice. The bodyweight of vaccinated hamster remained higher than those sham or vehicle-treated animals (FIG. 11).

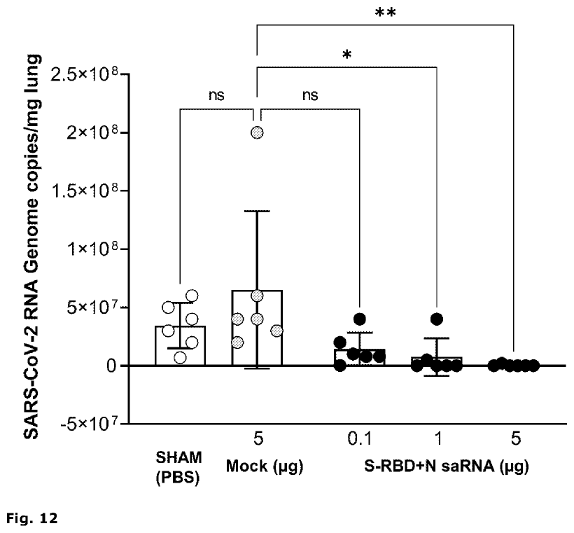

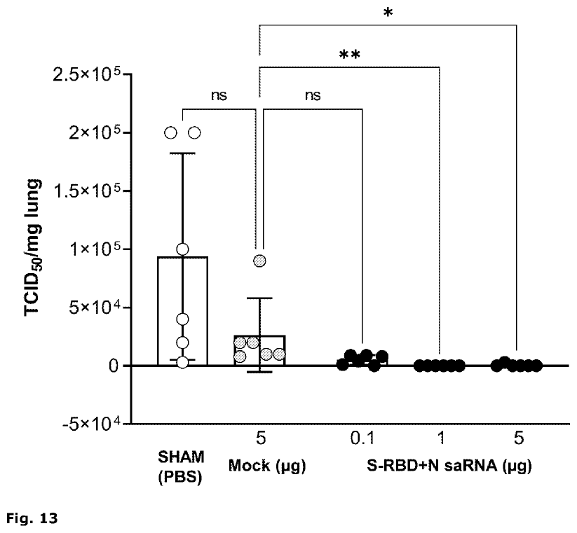

Infectious viral content was quantified and reported as the amount of SARS-CoV-2 RNA genome copies per mg lung tissue (FIG. 12). In addition, 50% tissue culture infective dose (TCID50) were determined as measure of infectious virus titer. This endpoint dilution assay quantifies the amount of virus required to produce a cytopathic effect in 50% of cells inoculated with serum from hamsters (FIG. 13). For both assays, a lower viral load after combined vaccination with 0.1 μg S-RBD+N saRNA was observed and significant reduction of virus after vaccination with 1 and 5 μg of the combined S-RBD+N saRNA vaccine.

After sacrifice (post challenge) cyto- and chemokine mRNA expression was analyzed in lung tissue via RT-qPCR. In the lung tissue of hamsters vaccinated with 1 μg and 5 μg S-RBD+N, IL-6 and IP-10 (CXCL10) mRNA expression levels are reduced compared to mock vaccinated control groups at 0.1 and 1 ug dose conditions for IL-6 and at 1 and 5 ug dose conditions for IL-10 (see FIGS. 15 & 16). This is of great importance, as IL-6 cytokine production by macrophages are elevated in COVID-19 patients, which induces a proinflammatory response. Similarly, the chemokine IP10 has also been associated with detrimental cytokine storm in COVID-19 infected patients.

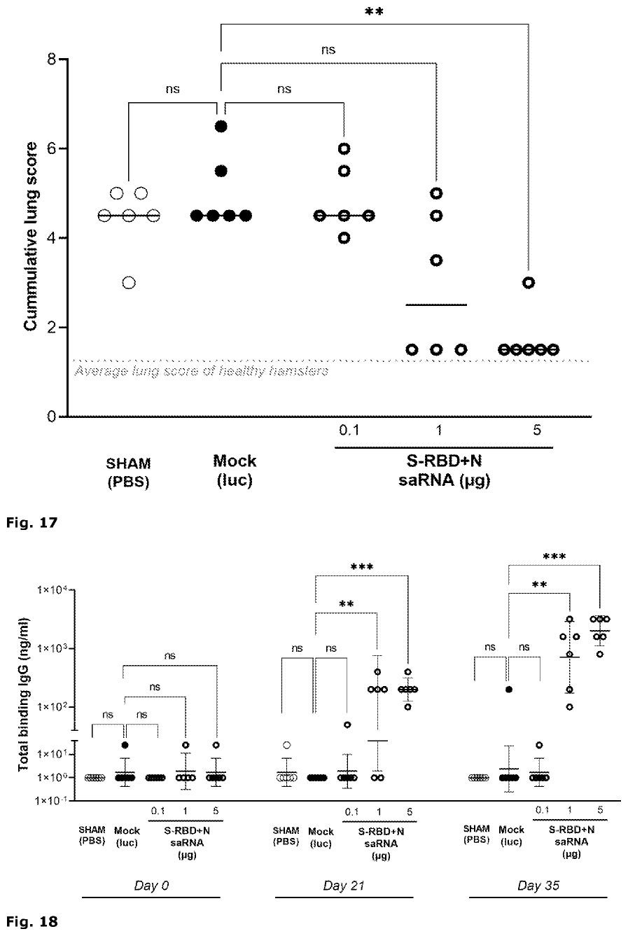

After sacrifice (post-challenge), lung tissue was removed for histopathological analysis to assess the impact of vaccination on the severity of lung disease (FIG. 17). Total lung histopathology score was calculated based on the alveolar damage (edema and hemorrhage), the presence of apoptotic bodies in bronchus wall & necrotizing bronchiolitis, perivascular edema and inflammation, peribronchial inflammation, endothelialitis, bronchopneumonia and % of lungs that were involved. After challenge, unvaccinated and mock (and 0.1 μg) vaccinated hamsters suffer from deteriorating lung pathology. This was prevented via sa-RNA-S-RBD+sa-RNA-N (ZIP1642) vaccination with a 1 and 5 μg prime-boost dosage regimen The total binding IgG antibody responses were measured by serum ELISA to assess the induction of humoral immunity after prime & boost vaccination with ZIP1642. Both prime and boost immunization with 1 μg and 5 μg of S-RBD+N saRNA significantly induced SARS-CoV-2 specific binding antibodies (FIG. 18).

### Example 8—Combined Immunization with LNP Formulated Sa-RNA-S-RBD and sa-RNA-N Elicits T Cell and Cytokine Responses in Mice

The humoral immune response against SARS-CoV-2 can be influenced by SARS-CoV-2-specific T-cell immunity, which can elicit either protective or damaging effects during the recovery of COVID-19 patients, depending on the nature of the mobilized T cells. Activated CD4+ T cells are critical for B-cell activation and antibody production and can be segregated into functional subsets based on their cytokine production. Recent studies revealed that Th1 CD4+ T cell responses are associated with effective resolution of SARS-CoV-2 infection, whereas the induction of CD4+ Th2 cells have been associated with immunopathology. Furthermore, it has been demonstrated that vaccine-associated enhanced respiratory disease (VAERD) is generally not observed when a CD4+ Th1 response occurs in the absence of a Th2 response. Thus, vaccination strategies should elicit Th1 biased CD4+ T cell immunity against SARS-CoV-2.

After boost vaccination, combined antigen S-RBD+N saRNA (ZIP1642) immunization is able to induce superior CD4+ Th1 biased S-specific T cell responses compared to immunization against the S-RBD antigen alone. In addition, induction of CD8+CTL responses are expected. Such CD8+CTL responses have recently been suggested to play a protective role in mild COVID-19 disease. Both CMI responses are expected to occur in the absence of long-term cytokine expression.

CD8+CTL toxicity assays, are performed. Flow cytometry experiments demonstrate the induction of CD8+CTL responses, based on cell count. T cell functionality assays, demonstrate the cytotoxic potential (and thus effectiveness) of these CD8+ cells. The S-specific T cell response is shown in FIG. 20.

To conclude,

Flow cytometry & functionality assay show that a CD8+CTL response that is functional against SARS-CoV-2 infection.

Cytokine responses are an indication of the induction of the aspecific innate immunity. After vaccination, high cytokine responses are reponsible for side effects, such as fever and muscle pain. Preferably these are to be limited. Preliminary data (not disclosed) suggest the decrease of cytokine levels after vaccination.

Finally, the IgG2/IgG1 ratios are greater in vaccinated mice, indicating the preferred Th1 biased response. IL-4 regulates B cells for secretion of IgG1 antibodies, whereas interferon-y stimulates the expression of IgG2a antibodies, hence rendering either isotype an indicator of the underlying Th2 (IL-4) or Th1 (IFNγ) response in mice.

To conclude, results in vaccinated mice show an efficient response and suggest long-term protection against SARS-CoV-2 infection.

### Example 9 VEEV-Based saRNA Platform

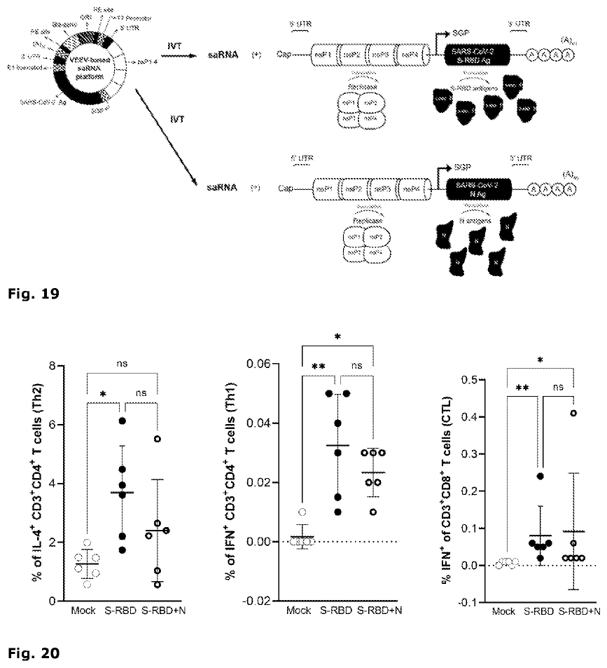

FIG. 19 shows a possible embodiment of a vector to be used in the context of the current invention as well as the combination of two self-amplifying RNA strands that can be used for the production of a specific RNA vaccine. SARS-CoV-2 Receptor-Binding Domain fused to C3d-p28.6 carrying TC-83 strain genome is used as replicon backbone to drive self-amplifying RNA expression (FIG. 19). Both conventional and self-amplifying mRNAs share basic elements including a cap, 5′ UTR, 3′ UTR, and poly(A) tail of variable length. Self-amplifying RNA (saRNA) also encode four non-structural proteins (nsP1-4) and a subgenomic promoter (SGP) derived from the genome of the alphavirus. nsP1-4 encode a replicase responsible for amplification of the saRNA that enable lower doses than non-replicating mRNA. The aforementioned backbone may further comprise a 5′ cap (e.g., 7mG(5′)ppp(5′)NImpNp). The backbone according to the current invention preferably comprises an A3G mutation in the 5′UTR and/or a Q739L mutation in Nonstructural Protein 2 (nsP2). A sequence encoding the Spike protein antigen is cloned after the SGP promoter, whereby the Spike protein antigen is a truncated form of the Spike protein comprising the Receptor-Binding Domain (RBD). Alternatively, a sequence encoding SARS-CoV-2 Nucleocapsid protein antigen is cloned after the SGP promoter. Following delivery to the cytoplasm, translation of the saRNA produces the non-structural proteins 1-4 (nsP 1-4) that form the RNA-dependent RNA polymerase (RDRP). RDRP is responsible for replication of the saRNA producing copies of the saRNA. Multiple copies of the subgenomic RNA are hence produced from each saRNA originally delivered. This leads to translation of many more copies of the antigen when compared to a non-amplifying RNA. The vector used to produce the RNA is equally shown in FIG. 19. Said vector comprises preferably an antigen, wherein said antigen sequence encodes for a SARS-CoV-2 Spike protein or a truncated form thereof, or wherein said antigen sequence encodes for a SARS-CoV-2 Nucleocapsid (N) antigen and wherein said antigen is downstream of a promoter sequence, preferably an alphavirus derived subgenomic promoter (SGP).

Said vector further comprises a poly(A) sequence downstream of said antigen sequence; and sequences encoding for non-structural proteins nsP1 to 4 of the Venezuelan Equine Encephalitis Virus. Preferably, the sequence of nsP2 is such that it encodes for an nsP2 protein having an A3G mutation in the 5′UTR and/or a Q739L mutation.

Said vector may further comprise an origin of replication, and promoter sequences such as a T7 or SP6 promoter. The vector may comprise a selection gene, eg encoding an antibiotic, for the purpose of producing the vector in an expression system.

The vector can be plasmid DNA or linearized DNA. For that purpose, a plasmid may be comprised of a restriction enzyme (RE) site allowing the linearization of said plasmid.

### Example 10—Vector and Constructs According to an Embodiment of the Current Invention

FIG. 19 shows a schematic representation of a possible embodiment of a vector to be used in the context of the current invention as well as the self-amplifying RNA strands that can be used for the production of a specific RNA SARS-CoV-2 vaccine. The vaccine allows for the immunization with several SARS-CoV-2 antigens, that are linked to multiple identified variants. As such, a more efficient and better protection against disease is obtained.

The RNA strands can be produced by means of DNA vector such as a plasmid. Said vector can be equipped with conventional regulatory regions such as an origin of replication for allowing replication, eg in a prokaryotic system. The vector may further be provided with nucleotide sequences encoding for proteins allowing selection, such as for instance sequences encoding for a resistance gene. The vector may further be provided with restriction enzyme sites, for cloning purposes and for allowing linearization of the vector. Promoter regions such as T7 (or SP6, not shown) can be present for allowing in vitro RNA transcription.

In the example given in FIG. 19 and downstream from the T7 promoter, the vector comprises, in 5′ to 3′ order (i) a 5′UTR sequence, (ii) nucleotide sequence(s) encoding for Venezuelan equine encephalitis virus, nonstructural proteins nsP1, nsP2, nsP3, and nsP4, (iii) a SGP promoter region which is operably linked to a nucleic acid sequence encoding a SARS-CoV-2 Nucleocapsid protein antigen or a nucleic acid sequence encoding a (truncated) SARS-CoV-2 Spike protein antigen comprising the RBD region, (iv) a 3′UTR and (v) a polyadenylate tract.

The vector as shown in FIG. 19 is used for the in vitro transcription of an mRNA strand. Said mRNA strand is capped at its 5′UTR and further comprises (in the 5′ to 3′ order):
A 5′ UTR;
Sequences encoding for Venezuelan equine encephalitis virus, nonstructural proteins nsP1, nsP2, nsP3, and nsP4;
A subgenomic promoter region
A sequence encoding for a SARS-CoV-2 antigen
A 3′UTR
A polyadenylate tract

A plurality of these mRNA strands will be formulated as a vaccine and can be delivered to a subject by conventional routes. Once administered to said subject, and following in situ translation, the nsP1-4 proteins will form a RNA-dependent RNA polymerase (RdRP) complex which on its turn will start amplifying mRNA transcripts from the SGP regions in the mRNA. The latter results in multiple copies of the subgenomic RNA from each saRNA originally delivered.

A subgenomic RNA transcript (5) will at its 5′ region be capped by the capping activity present in the replicase. The coding sequence of the N protein or S protein present in the transcript will be translated, resulting in the production of N or S antigen in the cell.

1. A pharmaceutical composition comprising
a sequence encoding a SARS-CoV-2 Spike protein antigen and a sequence encoding a SARS-CoV-2 Nucleocapsid antigen, wherein the sequence encoding the SARS-CoV-2 Spike protein antigen and the sequence encoding the SARS-CoV-2 Nucleocapsid protein antigen are comprised in an effective amount of; and
a pharmaceutically acceptable carrier and/or an acceptable pharmaceutically acceptable vehicle.

2. The pharmaceutical composition of claim 1, wherein the sequence encoding the 15 SARS-CoV-2 Spike protein antigen and the sequence encoding the SARS-CoV-2 Nucleocapsid protein antigen are comprised in the same self-replicating RNA molecule, or wherein the sequence encoding the SARS-CoV-2 Spike protein antigen and the sequence encoding the SARS-CoV-2 Nucleocapsid protein antigen are comprised in different self-replicating RNA molecules.

3. The pharmaceutical composition of claim 1, wherein said self-replicating RNA molecules are derived from an alphavirus and comprise a sequence encoding for nonstructural alphavirus proteins.

4. The pharmaceutical composition of claim 3, wherein the alphavirus is a Venezuelan Equine Encephalitis Virus (VEEV), such as strain TC-83 or a strain having at least 90% sequence identity.

5. The pharmaceutical composition of claim 3, wherein the one or more self-replicating RNA molecules comprise a 5′UTR having an A3G mutation in said 5′UTR and/or a Nonstructural Protein 2 (nsP2) having a Q739L mutation.

6. The pharmaceutical composition of claim 1, wherein the Spike protein antigen is a truncated form of the Spike protein comprising the Receptor-Binding Domain (RBD) of the Spike protein.

7. The pharmaceutical composition of claim 6, wherein the RBD corresponds to SEQ ID NO: 1 or an amino acid sequence having at least 95% identity.

8. The pharmaceutical composition of claim 1 wherein said sequence encoding the SARS-CoV-2 Spike protein antigen comprises:
a 5′ cap, followed by
a sequence encoding nonstructural alphavirus proteins nsP1, nsP2, nsP3 and nsP4;
a subgenomic promoter followed by
a sequence encoding for the SARS-CoV-2 Spike protein antigen; and
a poly-A tail downstream of said SARS-CoV-2 Spike protein antigen.

9. The pharmaceutical composition of claim 1 wherein said sequence encoding a SARS-CoV-2 Nucleocapsid (N) antigen comprises:
a 5′ cap, followed by
a sequence encoding nonstructural alphavirus proteins nsP1, nsP2, nsP3 and nsP4;
a subgenomic promoter followed by a sequence encoding for the SARS-CoV-2 N protein antigen; and
a poly-A tail downstream of said SARS-CoV-2 N protein antigen.

10. The pharmaceutical composition of claim 1, further comprising at least one adjuvant.

11. The pharmaceutical composition of claim 1, further comprising a cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, and a polycationic peptide, or a cationic nano-emulsion.

12. The pharmaceutical composition of claim 1, wherein the one or more self-replicating RNA molecules are encapsulated in, bound to or adsorbed on a cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, a cationic nano-emulsion and combinations thereof.

13. The pharmaceutical composition according claim 1 wherein said RNA molecules are encapsulated in, bound to or adsorbed on a cationic lipid, a lipid nanoparticle, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, a cationic nano-emulsion and combinations thereof and wherein the effective dose of said RNA in said vaccine is between 0.1 and 100 pg.

14. The pharmaceutical composition of claim 1 for use in inducing an immune response in a subject.

15. The pharmaceutical composition of claim 1, for use in vaccinating a subject against SARS-CoV-2.

16. The pharmaceutical composition for use according to claim 14 wherein an effective dose of said RNA is between 0.1 and 100 pg.

17. The pharmaceutical composition for use according to claim 14, wherein said composition is administered intramuscular, intradermal or subcutaneous.

18. The pharmaceutical composition for use according to claim 14, wherein said composition is administered as a single dose or as a multi-dose, requiring a series of two or more doses, administered within a pre-defined timespan.

19. The pharmaceutical composition for use according to claim 14, wherein said composition is administered periodically, such as annually or bi-annually.

20. The pharmaceutical composition for use according to claim 14, wherein a dose of said pharmaceutical composition is between 0.05 and 1 ml.
